(19) United States
(12) Patent Application Publication
(10) Pub. No.: US 2020/0002750 A1
(43) Pub. Date: Jan. 2, 2020

(54) METHODS FOR DETECTING C. CANIMORSUS CAPSULAR SEROTYPES IN A SAMPLE

(71) Applicant: UNIVERSITÉ DE NAMUR ASBL, Namur (BE)

(72) Inventors: Francesco Renzi, Namur (BE); Estelle Hess, Namur (BE); Guy Richard Cornelis, Crupet (BE)

(73) Assignee: UNIVERSITÉ DE NAMUR ASBL, Namur (BE)

(21) Appl. No.: 16/482,769

(22) PCT Filed: Feb. 16, 2018
(86) PCT No.: PCT/EP2018/053959

§ 371 Date: Aug. 1, 2019

(30) Foreign Application Priority Data
Feb. 17, 2017 (EP) 17156688.8

(51) Int. Cl. C12Q 1/689; C12Q 1/686

(52) U.S. Cl. CPC C12Q 1/689; C12Q 1/686

(57) ABSTRACT

Described herein are methods for identifying C. canimorsus in a sample as one of serotype A, serotype B, serotype C, serotype D or serotype E, amplification primer pairs, sets of primer pairs, oligonucleotide probes, sets of oligonucleotide probes, polyclonal antibodies, sets of polyclonal antibodies and kits that can be used for such methods. The application further provides polyvalent vaccines for the protection against an infection with C. canimorsus, methods of preparing said polyvalent vaccines and uses of these polyvalent vaccines.

### TECHNICAL FIELD

The present invention is situated in the field of in vitro detection methods for potentially pathogenic C. canimorsus strains. More particularly, the invention provides methods for detecting potentially dangerous C. canimorsus strains in a sample and sets of primer pairs and kits that can be used for such methods.

### BACKGROUND OF THE INVENTION

Capnocytophaga canimorsus (C. canimorsus) is part of the normal bacterial flora in the oral cavity of dogs and cats, together with Capnocytophaga cynodegmi and Capnocytophaga canis and is transmitted to humans after a bite, scratch or close contact. C. canimorsus are not reported to cause infections in dogs. However, C. canimorsus may cause rare but life-threatening infections in humans that are in contact with dogs and/or cats. C. canimorsus infection in humans often evolves to a septic shock despite the administration of an adequate first-line antibiotic treatment. The prognosis of C. canimorsus sepsis is bad with significant morbidity among which extremities amputations, myocardial infarction, renal failure, and a mortality rate of 30%. Persons at increased risk of developing C. canimorsus infections include patients who have undergone a splenectomy and those who abuse alcohol. Nevertheless up to 40% of patients presented no obvious risk factor. The carriage of C. canimorsus in dogs can be estimated around 40% meaning that almost every second dog would be dangerous for humans. In spite of that, the frequency of human diseases is only in the range of 4 cases per millions humans per year. There is no explanation for this low frequency. One possibility could be that a small fraction of the strains of C. canimorsus is more dangerous for humans than others but this was not investigated thus far.

C. canimorsus is often difficult to isolate and identify. Diagnosis of a C. canimorsus infection is usually based on bacterial culture of blood or other bodily fluids. The bacterium grows slowly on special media like heart infusion agar with 5% sheep blood and may require an extended incubation period (several days), delaying laboratory reports and indirectly affecting therapy options and treatment. In some case reports even, although bacteria were visible in the blood of patients, all the cultures remained negative. As a result hereof, treatment for infection in severe cases needs to be started before there is time to confirm the diagnosis. Alternatively, the presence of C. canimorsus may be identified by 16S ribosomal DNA polymerase chain reaction (PCR) directly from a blood sample. However, current methods do not allow identifying in dogs and/or cats those C. canimorsus strains which are more dangerous to humans because it is not known if human infections are due to any C. canimorsus or to only a small subpopulation of C. canimorsus.

In view of the above, new approaches need to be developed to identify the C. canimorsus strains which are more dangerous to human thereby helping to prevent these life threatening infections in humans.

### SUMMARY OF THE INVENTION

The present inventors have found that not all C. canimorsus strains present in the normal bacterial flora of the oral cavity of dogs and/or cats are pathogenic for human subjects. More particularly, the Applicants found that C. canimorsus strains from clinical isolates (i.e. isolated from human subjects infected with C. canimorsus) are endowed with capsular polysaccharides (CPS) and that those polysaccharide structures present a limited variability, with 5 capsular serotypes (i.e. serotype A, serotype B, serotype C, serotype D and serotype E), of which 3 (i.e. serotype A, serotype B and serotype C) are found to be dominant in C. canimorsus strains from clinical isolates. In addition, a clear enrichment of those dominant capsular serotypes, namely serotype A, serotype B and serotype C, was found in the C. canimorsus strains isolated from patients as compared to the C. canimorsus strains isolated from dog mouths, implying that C. canimorsus strains endowed with a type A, B or C capsule are more virulent for humans.

The Applicants' finding that the clinical C. canimorsus strains are endowed with a CPS which presents a limited variability allowed them to further create identification methods that specifically discriminate between those C. canimorsus capsular serotypes. To be more specific, the Applicants found that C. canimorsus strains could be identified as one of serotype A, serotype B, serotype C, serotype D or serotype E (i.e. identified as a clinically relevant C. canimorsus strain) not only by immuno-chemistry using specific antisera but also by molecular genetics by amplifying a target nucleic acid region in one or more genes of the CPS/LOS biosynthesis and transport loci of C. canimorsus specific to C. canimorsus strains of serotype A, serotype B, serotype C, serotype D or serotype E. Moreover, the polyclonal antibodies engineered by the Applicant could be used for detecting and/or identifying C. canimorsus in a sample, such as a urine sample, as one of serotype A, serotype B, serotype C, serotype D or serotype E, by contacting the sample with said polyclonal antibodies, such as through an immunoassay employing said polyclonal antibodies. Also, the Applicants provide a polyvalent vaccine which may protect against at least the C. canimorsus strains which are the most dangerous to human (i.e. at least against C. canimorsus strains identified as one of serotype A, serotype B, or serotype C).

Accordingly, provided herein is a method for serotyping C. canimorsus in a sample, said method comprising the step of: contacting said sample or at least a portion of nucleic acids isolated from said sample under conditions conducive to polymerase-based nucleic acid amplification with an amplification primer pair configured to amplify a target nucleic acid region in a first gene (gene A) of the CPS/LOS biosynthesis and transport loci of C. canimorsus wherein said gene A has syntenic orthologs in C. canimorsus 1, 2, 3, 5, 10, 13, 15, 21, 22, 24 and 25; an amplification primer pair configured to amplify a target nucleic acid region in a second gene (gene B) of the LOS/capsule biosynthesis and transport loci of C. canimorsus wherein said gene B has syntenic orthologs in C. canimorsus 6, 8, 11, 16, 17, 18 and 23; an amplification primer pair configured to amplify a target nucleic acid region in a third gene (gene C) of the LOS/capsule biosynthesis and transport loci of C. canimorsus wherein said gene C has syntenic orthologs in C. canimorsus 9, 14, 19 and 20; an amplification primer pair configured to amplify a target nucleic acid region in a fourth gene (gene D) of the CPS/LOS biosynthesis and transport loci of C. canimorsus wherein said gene D has syntenic orthologs in C. canimorsus 7 and 12; an amplification primer pair configured to amplify a target nucleic acid region in a fifth gene (gene E) of the CPS/LOS biosynthesis and transport loci of C. canimorsus, wherein said gene E is unique to C. canimorsus 4; and/or an amplification primer pair configured to amplify a target nucleic acid region in a sixth gene (gene ABC) of the CPS/LOS biosynthesis and transport loci of C. canimorsus wherein said gene ABC has syntenic orthologs in C. canimorsus 1, 2, 3, 5, 6, 8, 9, 10, 11, 13, 14, 15, 16, 17, 18, 19, 20, 21, 22, 23, 24 and 25; and one or more of the following steps: detecting the amplified target nucleic acid region in said gene A, and optionally simultaneously not detecting the amplified target nucleic acid region in said gene B, C, D and E, whereby the presence of C. canimorsus capsular serotype A in the sample is detected; detecting the amplified target nucleic acid region in said gene B, and simultaneously not detecting the amplified target nucleic acid region in said gene A, C, D and E, whereby the presence of C. canimorsus capsular serotype B in the sample is detected; detecting the amplified target nucleic acid region in said gene C, and simultaneously not detecting the amplified target nucleic acid region in said gene A, B, D and E, whereby the presence of C. canimorsus capsular serotype C in the sample is detected; detecting the amplified target nucleic acid region in said gene D, and simultaneously not detecting the amplified target nucleic acid region in said gene A, B, C, E and ABC, whereby the presence of C. canimorsus capsular serotype D in the sample is detected; detecting the amplified target nucleic acid region in the fifth gene, and simultaneously not detecting the amplified target nucleic acid region in said gene A, B, C, D and ABC, whereby the presence of C. canimorsus capsular serotype E in the sample is detected; and/or detecting the amplified target nucleic acid region in said gene ABC, and simultaneously not detecting the amplified target nucleic acid region in said gene D and E, whereby the presence of C. canimorsus capsular serotype A, B and/or C in the sample is detected. In particular embodiments, said gene A is A4GalT-like glycosyltransferase gene (A4galT GT), preferably wherein A4galT GT has a coding nucleic acid sequence that is at least 90%, preferably at least 95%, more preferably 100% identical to SEQ ID NO: 1; said gene B is a first family 1 glycosyltransferase gene (GT1), preferably wherein said first GT1 has a coding nucleic acid sequence that is at least 90%, preferably at least 95%, more preferably 100% identical to SEQ ID NO: 2 and/or 49; said gene C is wzy, preferably wherein wzy has a coding nucleic acid sequence that is at least 90%, preferably at least 95%, more preferably 100% identical to SEQ ID NO: 3; said gene D is wbbJ, preferably wherein wbbJ has a coding nucleic acid that is at least 90%, preferably at least 95%, more preferably 100% identical to SEQ ID NO: 4, said gene E is a second GT1, preferably wherein said second GT1 has a coding nucleic acid sequence that is at least 90%, preferably at least 95%, identical to SEQ ID NO: 5; and said gene ABC is wfdR, preferably wherein wfdR has a coding nucleic acid sequence that is at least 90%, preferably at least 95%, more preferably 100% identical to SEQ ID NO: 6, 7 and/or 8.

In particular embodiments, the amplification primer pair configured to amplify a target nucleic acid region in A4galT GT comprises a first amplification primer which comprises a nucleotide sequence which is at least 90%, preferably at least 95%, identical to SEQ ID NO: 9 or SEQ ID NO: 11 and a second amplification primer which comprises a nucleotide sequence which is at least 90%, preferably at least 95%, identical to SEQ ID NO: 10 or SEQ ID NO: 12; the amplification primer pair configured to amplify a target nucleic acid region in the first GT1 comprises a first amplification primer which comprises a nucleotide sequence which is at least 90%, preferably at least 95%, identical to SEQ ID NO: 13 or SEQ ID NO: 15 and a second amplification primer which comprises a nucleotide sequence which is at least 90%, preferably at least 95%, identical to SEQ ID NO: 14 or SEQ ID NO: 16; the amplification primer pair configured to amplify a target nucleic acid region in wzy comprises a first amplification primer which comprises a nucleotide sequence which is at least 90%, preferably at least 95%, identical to SEQ ID NO: 17 and a second amplification primer which comprises a nucleotide sequence which is at least 90%, preferably at least 95%, identical to SEQ ID NO: 18; the amplification primer pair configured to amplify a target nucleic acid region in wbbJ comprises a first amplification primer which comprises a nucleotide sequence which is at least 90%, preferably at least 95%, identical to SEQ ID NO: 19 and a second amplification primer which comprises a nucleotide sequence which is at least 90%, preferably at least 95%, identical to SEQ ID NO: 20; the amplification primer pair configured to amplify a target nucleic acid region in the second GT1 comprises a first amplification primer which comprises a nucleotide sequence which is at least 90%, preferably at least 95%, identical to SEQ ID NO: 21 and a second amplification primer which comprises a nucleotide sequence which is at least 90%, preferably at least 95%, identical to SEQ ID NO: 22; and/or the amplification primer pair configured to amplify a target nucleic acid region in wfdR comprises a first amplification primer which comprises a nucleotide sequence which is at least 90%, preferably at least 95%, identical to SEQ ID NO: 23 and a second amplification primer which comprises a nucleotide sequence which is at least 90%, preferably at least 95%, identical to SEQ ID NO: 24.

In particular embodiments, the polymerase-based nucleic acid amplification is multiplexed, such that at least two at least three, at least four or at least five of the target nucleic acid regions are amplified in the same polymerase-based nucleic acid amplification reaction.

In particular embodiments, the polymerase-based nucleic acid amplification is polymerase chain reaction (PCR).

In particular embodiments, said sample is subjected to an immuno-purification step prior to said polymerase-based nucleic acid amplification. More particularly, the immune-purification step is performed using one or more polyclonal antibodies as envisaged herein, i.e, polyclonal antibodies capable of binding to specific serotypes of Capnocytophaga, more particularly of C. canimorsus. Also provided herein is a set of amplification primer pairs suitable for polymerase-based nucleic acid amplification, comprising an amplification primer pair configured to amplify a target nucleic acid region in a first gene (gene A) of the CPS/LOS biosynthesis and transport loci of C. canimorsus wherein said gene A has syntenic orthologs in C. canimorsus 1, 2, 3, 5, 10, 13, 15, 21, 22, 24 and/or 25; an amplification primer pair configured to amplify a target nucleic acid region in a second gene (gene B) of the LOS/capsule biosynthesis and transport loci of C. canimorsus wherein said gene B has syntenic orthologs in C. canimorsus 6, 8, 11, 16, 17, 18 and/or 23; an amplification primer pair configured to amplify a target nucleic acid region in a third gene (gene C) of the LOS/capsule biosynthesis and transport loci of C. canimorsus wherein said gene C has syntenic orthologs in C. canimorsus 9, 14, 19 and/or 20; an amplification primer pair configured to amplify a target nucleic acid region in a fourth gene (gene D) of the CPS/LOS biosynthesis and transport loci of C. canimorsus wherein said gene D has syntenic orthologs in C. canimorsus 7 and 12; an amplification primer pair configured to amplify a target nucleic acid region in a fifth gene (gene E) of the CPS/LOS biosynthesis and transport loci of C. canimorsus, wherein said gene E is unique to C. canimorsus 4; and/or an amplification primer pair configured to amplify a target nucleic acid region in a sixth gene (gene ABC) of the CPS/LOS biosynthesis and transport loci of C. canimorsus wherein said gene ABC has syntenic orthologs in C. canimorsus 1, 2, 3, 5, 6, 8, 9, 10, 11, 13, 14, 15, 16, 17, C. canimorsus 18, C. canimorsus 19, 20, 21, 22, C. canimorsus 23, 24 and/or 25.

In particular embodiments, said gene A is C. canimorsus A4galT GT, wherein the coding nucleic acid sequence of said gene is at least 90%, preferably at least 95%, more preferably 100% identical to SEQ ID NO: 1; said gene B is C. canimorsus GT1, wherein the coding nucleic acid sequence of said gene is at least 90%, preferably at least 95%, more preferably 100% identical to SEQ ID NO: 2 and/or 49; said gene C is C. canimorsus wzy, wherein the coding nucleic acid sequence of said gene is at least 90%, preferably at least 95%, more preferably 100% identical to SEQ ID NO: 3; said gene D is C. canimorsus wbbJ; wherein the coding nucleic acid sequence of said gene is at least 90%, preferably at least 95%, more preferably 100% identical to SEQ ID NO: 4; said gene E is a second C. canimorsus GT1, wherein the coding nucleic acid sequence of said gene is at least 90%, preferably at least 95%, identical to SEQ ID NO: 5 and/or said gene ABC is C. canimorsus wfdR, wherein the coding nucleic acid sequence of said gene is at least 90%, preferably at least 95%, more preferably 100% identical to SEQ ID NO: 6, SEQ ID NO:7 and/or SEQ ID NO:8.

In particular embodiments, the amplification primer pair configured to amplify a target nucleic acid region in A4galT GT comprises a first amplification primer which comprises a nucleotide sequence which is at least 90%, preferably at least 95%, identical to SEQ ID NO: 9 or 11 and a second amplification primer which comprises a nucleotide sequence which is at least 90%, preferably at least 95%, identical to SEQ ID NO: 10 or 12; the amplification primer pair configured to amplify a target nucleic acid region in the first GT1 comprises a first amplification primer which comprises a nucleotide sequence which is at least 90%, preferably at least 95%, identical to SEQ ID NO: 13 or 15 and a second amplification primer which comprises a nucleotide sequence which is at least 90%, preferably at least 95%, identical to SEQ ID NO: 14 or 16; the amplification primer pair configured to amplify a target nucleic acid region in wzy comprises a first amplification primer which comprises a nucleotide sequence which is at least 90%, preferably at least 95%, identical to SEQ ID NO: 17 and a second amplification primer which comprises a nucleotide sequence which is at least 90%, preferably at least 95%, identical to SEQ ID NO: 18; the amplification primer pair configured to amplify a target nucleic acid region in wbbJ comprises a first amplification primer which comprises a nucleotide sequence which is at least 90%, preferably at least 95%, identical to SEQ ID NO: 19 and a second amplification primer which comprises a nucleotide sequence which is at least 90%, preferably at least 95%, identical to SEQ ID NO: 20; the amplification primer pair configured to amplify a target nucleic acid region in the second GT1 comprises a first amplification primer which comprises a nucleotide sequence which is at least 90%, preferably at least 95%, identical to SEQ ID NO: 21 and a second amplification primer which comprises a nucleotide sequence which is at least 90%, preferably at least 95%, identical to SEQ ID NO: 22; and/or the amplification primer pair configured to amplify a target nucleic acid region in wfdR comprises a first amplification primer which comprises a nucleotide sequence which is at least 90%, preferably at least 95%, identical to SEQ ID NO: 23 and a second amplification primer which comprises a nucleotide sequence which is at least 90%, preferably at least 95%, identical to SEQ ID NO: 24.

In particular embodiments, the amplification primer pairs are configured to allow for multiplexed polymerase-based nucleic acid amplification, such that at least two, at least three, at least four or at least five of the target nucleic acid regions can be amplified in the same polymerase-based nucleic acid amplification reaction.

Also provided herein is a kit of parts comprising the set of amplification primer pairs as taught, and optionally further comprising reagents sufficient for formulating a polymerase-based nucleic acid amplification reaction mixture.

Also, the application provides a polyclonal antibody recognizing capsular polysaccharides (CPS) and/or lipooligosaccharides (LOS) of wild-type C. canimorsus bacteria of one or more but not all capsular serotypes which is obtainable by adsorbing anti-serum obtained by immunization of a non-human animal with a composition comprising wild-type C. canimorsus bacteria of said one or more capsular serotypes subsequently or simultaneously with one or more wild type C. canimorsus strains selected from a wild-type C. canimorsus strain of the capsular serotype A, a wild-type C. canimorsus strain of the capsular serotype B, a wild-type C. canimorsus strain of the capsular serotype C, a wild-type C. canimorsus strain of the capsular serotype D and a wild-type C. canimorsus strain, of the capsular serotype E, but not with wild-type C. canimorsus strains, of the one or more capsular serotypes used for immunization. Accordingly, provided herein is a polyclonal antibody which is obtained by adsorbing anti-serum obtained by immunization with wild-type C. canimorsus with non-capsulated and rough mutant C. canimorsus bacteria or by absorbing anti-serum obtained by immunization with wild-type C. canimorsus bacteria subsequently or simultaneously with at least one wild-type C. canimorsus strain of the capsular serotype A, at least one wild-type C. canimorsus strain of the capsular serotype B, at least one wild-type C. canimorsus strain of the capsular serotype C, at least one wild-type C. canimorsus strain of the capsular serotype D and at least one wild-type C. canimorsus strain of the capsular serotype E, with the proviso that if the anti-serum is adsorbed with wild-type C. canimorsus strains, the anti-serum is not adsorbed with wild-type C. canimorsus strains of the same capsular serotype as used for immunization; wherein said non-capsulated and rough mutant C. canimorsus bacteria are of the same serotype as the wild-type C. canimorsus bacteria used for immunization and wherein said polyclonal antibody specifically recognizes the capsular polysaccharides (CPS) and/or lipooligosaccharides (LOS) of wild-type C. canimorsus bacteria of the same capsular serotype as the wild-type C. canimorsus bacteria used for immunization. In particular embodiments, the polyclonal antibody additionally does not react with a non-capsular or rough mutant strain of one or more of C. canimorsus serotypes, more particularly, does not react with a non-capsular or rough mutant strain of the one or more C. canimorsus serotypes used for immunization. The application also provides methods for producing the antibodies disclosed herein which comprise immunization with one or more strains of one or more C. canimorsus serotypes and adsorption of the antibodies so obtained with one or more strains of one or more C. canimorsus serotypes other than those used for immunization and/or adsorption with one or more rough or non-capsular mutant strains, more particularly with a rough or non-capsular mutant strains of the serotype used for immunization.

It is noted that the inventors have found that different Capnocytophaga have the same capsular serotypes such that antibodies obtained by immunization of a non-human animal with a composition comprising wild-type C. canimorsus bacteria of one or more capsular serotypes (and further absorbed with at least one wild-type C. canimorsus strain of one or more capsular serotypes so as to get a capsular serotype-specific antiserum specific for one or more serotypes) will recognize other Capnocytophaga species of the same serotype.

Accordingly, the application provides polyclonal antibodies which specifically recognizing capsular polysaccharides (CPS) and/or lipooligosaccharides (LOS) of wild-type Capnocytophaga bacteria of one or more but not all capsular serotypes.

Also provided herein is a method for serotyping C. canimorsus in a sample, said method comprising detecting the presence of capsular polysaccharides (CPS) of C. canimorsus capsular serotype A, B, C, D and/or E in a sample through an immunoassay employing one or more polyclonal antibodies specifically recognizing CPS of the C. canimorsus capsular serotype A, B, C, D or E as taught herein.

Also provided herein is a method for specifically identifying C. canimorsus of serotype A, serotype B, serotype C, serotype D or serotype E in a sample, said method comprising a step of subjecting said sample to an immuno-purification step using one or more polyclonal antibodies as envisaged herein; and a step of identifying C. canimorsus by performing a C. canimorsus-specific PCR on said immuno-purified sample, preferably a C. canimorsus-specific 16S ribosomal DNA PCR. Also provided herein is a polyvalent vaccine for protection against an infection with C. canimorsus comprising inactivated or attenuated cells of a C. canimorsus strain of the serotype A selected from the list consisting of C. canimorsus 1, 2, 3, 5, 10, 13, 15, 21, 22, 24 and 25, or fragments thereof; inactivated or attenuated cells of a C. canimorsus strain of the serotype B selected from the list consisting of C. canimorsus 6, 8, 11, 16, 17, 18 and 23, or fragments thereof; inactivated or attenuated cells of a C. canimorsus strain of the serotype C selected from the list consisting of C. canimorsus 9, 14, 19 and 20, or fragments thereof; inactivated or attenuated cells of a C. canimorsus strain of the serotype D selected from the list consisting of C. canimorsus 12 and 7, or fragments thereof; and/or inactivated or attenuated cells of a C. canimorsus strain of the serotype E, wherein said C. canimorsus strain is C. canimorsus 4, or fragments thereof.

Also provided herein is a method of preparing the polyvalent vaccine as taught herein.

Also provided herein is the use of the polyvalent vaccine as taught herein in the prevention or elimination of a bacterial infection with a C. canimorsus strain in a non-human animal, wherein the method is not a method of treatment practiced on the animal body.

### BRIEF DESCRIPTION OF THE FIGURES

FIG. 1. Capsular serotyping of C. canimorsus clinical isolates. Western blot analysis of proteinase-K treated lysates of patient-isolated C. canimorsus strains using the following sera: Y1C12 adsorbed anti-Cc5 (A), anti-Cc6 (B), anti-Cc9 (C), anti-Cc12 (D) and anti-Cc4 (E). Non-capsulated and rough mutants Cc5 Y1C12, Cc6 ΔwbuB, Cc9 ΔwbuB and Cc12 ΔwbtA were used in panels A, B, C and D respectively. Numbers correspond to molecular mass markers in kDa.

FIG. 2. Analysis of high molecular weight polysaccharide structures from the dog-hosted strains found positive by enzyme-linked immunosorbent assay (ELISA) screening. Western blot analysis of proteinase K treated lysates of dog-isolated C. canimorsus strains using the following sera: Y1C12 adsorbed anti-Cc5 (A), Cc6 ΔwbuB adsorbed anti-Cc6 (B), Cc9 ΔwbuB adsorbed anti-Cc9 (C), Cc12 ΔwbtA adsorbed anti-Cc12 (D) and anti-Cc4 adsorbed with all clinical strains except Cc4 (E). Numbers correspond to molecular mass markers in kDa.

FIG. 3. Prevalence of capsular serotypes A to E in C. canimorsus strains isolated from patients or dogs. Summary of capsular serotype prevalence in strains isolated from patients (A) or dogs (B).

FIG. 4. PCR detection of capsular serotypes A, B, C, D and E in C. canimorsus strains isolated from patients and dogs. Detection of capsular serotypes in C. canimorsus and dog isolates using specific amplification primers. PCR A was performed using amplification primers according to SEQ ID NO. 11 and 12, PCR B was performed using amplification primers according to SEQ ID NO. 15 and 16, PCR C was performed using amplification primers according to SEQ ID NO. 17 and 18, PCR D was performed using amplification primers according to SEQ ID NO. 19 and 20, PCR E was performed using amplification primers according to SEQ ID NO. 21 and 22, and PCR ABC was performed using amplification primers according to SEQ ID NO. 23 and 24.

FIG. 5. Synteny analysis of lipooligosaccharide (LOS)/capsular polysaccharides (CPS) loci in the A, B, C, D, E capsular serotypes. Comparison of the CPS/LOS-biosynthesis and transport loci of the seven sequenced C. canimorsus strains. The boxes indicate different genomic loci. Orthologs of the Cc5 genes are indicated in grey. The target genes detected by the A-, B-, C-, D- and E-serotype specific PCR are indicated in black. The target genes detected by the ABC serotypes specific PCR are underlined. Genes indicated in white are strain specific genes likely involved in CPS/LOS biosynthesis. The hatched pattern indicates genes likely unrelated to CPS/LOS biosynthesis and transport. Fragmented genes are marked with (f). Note that the genomes of Cc2, Cc4, Cc6, Cc9, Cc11 and Cc12 are draft genomes. For the sake of simplicity genes are not represented to scale.

FIG. 6. Control of adsorption efficacy by immunofluorescence microscopy. Paraformaldehyde-fixed bacteria were stained using the following sera:Y1C12-adsorbed anti-Cc5 (A), Cc6 ΔwbuB-adsorbed anti-Cc6 (B), Cc9 ΔwbuB-adsorbed anti-Cc9 (C), Cc12 ΔwbtA-adsorbed anti-Cc12 and anti-Cc4 absorbed with all clinical strains except Cc4 (E). Bar, 10 μM.

FIG. 7. Specific detection of the B serotype. Alignment of the first 42 nucleotides of the glycosyl transferase 1 (GT1) genes amplified for the detection of the B serotype. Underlined is the sequence of the forward amplification primer for serotype B (SEQ ID NO: 15) that anneals to the target genes. Dashed lines indicate missing nucleotides in Cc2 and Cc9 GT1 while in grey are the mismatches.

FIG. 8. Analysis of capsular structures from the dog-hosted strains found positive by the PCR screening. Western blot analysis of proteinase-K treated lysates of the dog-isolated C. canimorsus strains: CcD57 using the Cc6 ΔwbuB adsorbed anti-Cc6 serum (A) and CcD10 using anti-Cc4 adsorbed with all clinical strains except Cc4 serum (B).

FIG. 9. Table 2. Coding nucleic acid sequences of A4galT GT, GT1, wzy, wbbJ and wfdR. The preferred oligonucleotides annealing regions are indicated in bold and are underlined.

FIG. 10. Table 3. Nucleic acid sequences of the target nucleic acid region in A4galT GT, GT1, wzy, wbbJ and wfdR.

FIG. 11. Table 7. Oligonucleotides used in examples 1 to 4.

FIG. 12. Control of the specificity of the anti-A serum (Y1C12 adsorbed anti-Cc5 serum). (a) Immunoblot analysis of proteinase K-treated wt Cc5 and Y1C12 mutant bacterial lysates and of pure LOS isolated from wt Cc5 and Y1C12 mutant using anti-Cc5 serum. (b) Immunoblot analysis as described in (a) using the Y1C12-adsorbed anti-Cc5 serum. Band A, B and D refer to uncharacterized structures, band E to the CPS, band C to the WT LOS and band C* to the mutant LOS. The bands of interest are band «E» (CPS) and band «C» (LOS). Numbers correspond to molecular mass markers in kDa.

FIG. 13. Control of the specificity of anti-B, anti-C, anti-D, and anti-E antisera. (A) Immunoblot analysis of proteinase K-treated bacterial lysates from Cc6 wt and wbuB mutant using anti-Cc6 or Cc6 wbuB adsorbed anti-Cc6 serum. (B) Immunoblot analysis of proteinase K-treated bacterial lysates from Cc9 wt and wbuB mutant using anti-Cc9 or Cc9 wbuB adsorbed anti-Cc9 serum. (C) Immunoblot analysis of proteinase K-treated bacterial lysates from Cc12 wt and wbtA mutant using anti-Cc12 or Cc12 wbtA adsorbed anti-Cc12 serum. (D) Immunoblot analysis of proteinase K-treated bacterial lysates from Cc4 wt using anti-Cc4 or anti-Cc4 adsorbed with 24 clinical isolates serum (including Cc1, Cc2, Cc3, Cc5, Cc6, Cc7, Cc8, Cc9, Cc10, Cc11, Cc12, Cc13, Cc14, Cc15, Cc16, Cc17, Cc18, Cc19, Cc20, Cc21, Cc22, Cc23, Cc24, Cc25) serum. Band A refers to uncharacterized structures, band E to the CPS and band C to the WT LOS. Numbers correspond to molecular mass markers in kDa.

FIG. 14. Control of the specificity of the adsorbed anti-sera by ELISA. Two different bleedings (bl; Bl1 and Bl2) of each anti-serum were adsorbed and tested by ELISA at a dilution of 1/2000. Positive control refers to the type strain of each serotype (Cc5, Cc6, Cc9 Cc12 and Cc4 for A, B, C, D and E, respectively). Negative controls refers to the strains used to adsorb the anti-sera (Y1C12, Cc6 ΔwbuB, Cc9 ΔwbuB, Cc12 ΔwbtA and a mix of 24 clinical isolates (including Cc1, Cc2, Cc3, Cc5, Cc6, Cc7, Cc8, Cc9, Cc10, Cc11, Cc12, Cc13, Cc14, Cc15, Cc16, Cc17, Cc18, Cc19, Cc20, Cc21, Cc22, Cc23, Cc24, Cc25) for serotype A, B, C, D and E respectively).

### DETAILED DESCRIPTION OF THE INVENTION

Before the present C. canimorsus serotyping methods or methods for identifying C. canimorsus serotypes as well as tools for doing so are described, it is to be understood that the present C. canimorsus serotyping methods or methods for identifying C. canimorsus in a sample as one of serotype A, serotype B, serotype C, serotype D or serotype E, as well as tools for doing so are not limited to particular serotyping methods or tools/products, as such particular serotyping methods or methods for identifying C. canimorsus in a sample as one of serotype A, serotype B, serotype C, serotype D or serotype E, and tools may vary. It is also to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

In this specification and the appended claims, the singular forms “a”, “an”, and “the” include plural references unless the context clearly dictates otherwise.

The terms “comprising”, “comprises” and “comprised of”, as used herein, are synonymous with “including”, “includes” or “containing”, “contains”, and are inclusive or open-ended and do not exclude additional, non-recited members, elements or method steps.

The terms “comprising”, “comprises” and “comprised of” also include the term “consisting of”.

The term “about”, as used herein, when referring to a measurable value such as a parameter, an amount, a temporal duration, and the like, is meant to encompass variations of +/−10% or less, preferably +/−5% or less, more preferably +/−1% or less, and still more preferably +1-0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The Applicants have found that C. canimorsus strains from clinical isolates (i.e. isolated from human subjects infected with C. canimorsus) are endowed with capsular polysaccharides (CPS) and that those polysaccharide structures present a limited variability, with 5 capsular serotypes (i.e. serotype A, serotype B, serotype C, serotype D and serotype E), of which 3 (i.e. serotype A, serotype B and serotype C) are found to be dominant. In addition, a clear enrichment of those dominant capsular serotypes was found in the C. canimorsus strains isolated from patients as compared to the C. canimorsus strains isolated from dog mouths, implying that C. canimorsus strains endowed with a type A, B or C capsule are more virulent for humans. Accordingly, a few capsular serotypes appear to be more dangerous for humans than others.

The applicants' finding that the clinically relevant C. canimorsus strains are endowed with a CPS which presents a limited variability has allowed them to further create identification methods that specifically discriminate between those C. canimorsus capsular serotypes that are pathogenic to humans and those that are not. The Applicants identified the CPS/LOS biosynthesis and transport loci of the 5 C. canimorsus capsular serotypes and designed amplification primer pairs to specifically discriminate between them. Accordingly, described herein is a PCR typing method based on the amplification of specific genes of the CPS/LOS biosynthesis and transport loci, amplification primers, primer pairs and kits which can be used to serotype C. canimorsus strains, or to identify C. canimorsus strains as one of serotype A, serotype B, serotype C, serotype D or serotype E, in a sample from a subject known to harbour or suspected of harbouring C. canimorsus and thereby detect potentially pathogenic C. canimorsus in a sample, such as a sample of saliva isolated from dogs or cats. Accordingly, capsular serotyping of C. canimorsus strains or identifying C. canimorsus strains as one of serotype A, serotype B, serotype C, serotype D or serotype E, could contribute to the prevention of these infections. For example, holders of dogs and cats could take measurements to prevent scratches and/or bites of dogs/cats identified to carry potentially dangerous C. canimorsus strains, thereby preventing a potential C. canimorsus infection. Additionally, described herein are specific polyclonal antibodies against C. canimorsus capsular antigens and methods for detecting and/or serotyping C. canimorsus and/or identifying C. canimorsus strains as one of serotype A, serotype B, serotype C, serotype D or serotype E, in a sample, such as a blood or urine sample, preferably a urine sample, comprising detecting the presence of C. canimorsus capsular polysaccharides in a sample through an immunoassay employing said one or more polyclonal antibodies. This method could be implemented for rapidly detecting a C. canimorsus-derived capsular antigen at an early stage of infection and reduce the time for identifying the causal bacterium for sepsis, fever of unknown origin, meningitis, septic shock, respiratory tract infections, etc. Moreover, this method may be non-invasive, since a capsular antigen can be detected in a urine sample.

It is noted that the inventors have found that different Capnocytophaga have the same capsular serotypes such that antibodies obtained by immunization of a non-human animal with a composition comprising wild-type C. canimorsus bacteria of one or more capsular serotypes (and further absorbed with non-capsular or mutant bacteria so as to get a capsular serotype-specific antiserum specific for one or more serotypes) may recognize other Capnocytophaga species of the same serotype. Accordingly, the application provides polyclonal antibodies which specifically recognizing capsular polysaccharides (CPS) and/or lipooligosaccharides (LOS) of wild-type Capnocytophaga bacteria of one or more but not all capsular serotypes. However, given that in particular embodiments, the primary interest is in detecting the relevant pathogenic serotypes of C. canimorsus, it will be understood by the skilled person that in order to identify the presence of C. canimorsus of a particular serotype in a sample, it can in fact be sufficient to detect the presence of a C. canimorsus of a given serotype in sample. Where it is important to specifically identify (and optionally quantify the presence of the C. canimorsus serotype, further identification can be ensured as envisaged herein.

In addition, the high prevalence of a limited number of C. canimorsus serotypes among the human isolates sets the basis for a prophylactic vaccination of persons at risk. A polyvalent vaccine made of only 5 (i.e. serotype A, serotype B, serotype C, serotype D and serotype E), or even only 3 (i.e. serotype A, serotype B and serotype C), different C. canimorsus capsules may protect at least against the C. canimorsus strains which are the most dangerous to human. A vaccination of people at risk can thus be envisioned. Accordingly, provided herein are vaccines to help in the prevention of these dramatic C. canimorsus infections.

More particularly, present inventors have shown that C. canimorsus strains can be subdivided into capsular serotypes A, B, C, D and E (see Table 1) by looking at the presence of certain genes of the lipooligosaccharide (LOS)/CPS biosynthesis and transport loci (e.g. genes (presumed to be) involved in the biosynthesis of CPS).

Accordingly, the application provides methods for serotyping C. canimorsus in a sample, or for identifying C. canimorsus in a sample as one of serotype A, serotype B, serotype C, serotype D or serotype E, said method comprising the step of determining the presence and/or expression of a target nucleic acid region in a gene A, a gene B, a gene C, a gene D, a gene E and/or gene ABC of the CPS/LOS biosynthesis and transport loci of C. canimorsus in said sample and serotyping C. canimorsus or identifying C. canimorsus as one of serotype A, serotype B, serotype C, serotype D or serotype E in said sample, wherein.
the presence and/or expression of a target nucleic acid region in said gene A and optionally the absence and/or absence of expression of a target nucleic acid region in said genes B, C, D and E is indicative of the presence of a C. canimorsus of C. canimorsus capsular serotype A in said sample;
the presence and/or expression of a target nucleic acid region in said gene B, and the absence and/or absence of expression of a target nucleic acid region in said genes A, C, D and E is indicative of the presence of a C. canimorsus of C. canimorsus capsular serotype B in said sample;
the presence and/or expression of a target nucleic acid region in said gene C, and the absence and/or absence of expression of a target nucleic acid region in said genes A, B, D and E is indicative of the presence of a C. canimorsus of C. canimorsus capsular serotype C in said sample
the presence and/or expression of a target nucleic acid region in said gene D, and the absence and/or absence of expression of a target nucleic acid region in said genes A, B, C, and E is indicative of the presence of a C. canimorsus of C. canimorsus capsular serotype D in said sample
the presence and/or expression of a target nucleic acid region in said gene E, and the absence and/or absence of expression of a target nucleic acid region in said genes A, B, C, and D is indicative of the presence of a C. canimorsus of C. canimorsus capsular serotype E in said sample
the presence and/or expression of a target nucleic acid region in said gene ABC, and the absence and/or absence of expression of a target nucleic acid region in said genes D and E is indicative of the presence of a C. canimorsus of C. canimorsus capsular serotype A, B and/or C in said sample.

In particular embodiments, the methods comprise determining the presence and/or expression of a target nucleic acid region identified herein as A, B, C, D and E genes. However, it will be clear to the skilled person that, based on the identification of the pathogenic strains, other genes that are common to these pathogenic strains can easily be identified. Thus, while the application provides suitable A, B, C, D and E genes for use in this context, the identification of additional genes may be envisaged. Accordingly, the application also provides methods for identifying tools suitable for the detection of a pathogenic serotype in a sample, said method comprising
identifying a target nucleic acid region in a gene of the CPS/LOS biosynthesis and transport loci of C. canimorsus wherein said gene is referred to herein as a gene A and has syntenic orthologs in C. canimorsus strains Cc1, Cc2, Cc3, Cc 5, Cc10, Cc13, Cc15, Cc21, Cc22, Cc24 and Cc25 and not in C. canimorsus strains Cc4, Cc6, Cc7, Cc8, Cc9, Cc11, Cc12, Cc14, Cc16, Cc17, Cc18, Cc19, Cc20 and Cc23;
identifying a target nucleic acid region in a gene of the LOS/capsule biosynthesis and transport loci of C. canimorsus wherein said gene is referred to herein as a gene B and has syntenic orthologs in C. canimorsus strains Cc6, Cc8, Cc11, Cc16, Cc17, Cc18 and Cc23 and not C. canimorsus strains Cc1, Cc2, Cc3, Cc4, Cc5, Cc7, Cc9, Cc10, Cc12, Cc13, Cc14, Cc15, Cc19, Cc20, Cc21, Cc22, Cc24 and Cc25;
identifying a target nucleic acid region in a gene of the LOS/capsule biosynthesis and transport loci of C. canimorsus wherein said gene is referred to herein as a gene C and has syntenic orthologs in C. canimorsus strains Cc9, Cc14, Cc19 and Cc20 and not in C. canimorsus strains Cc1, Cc2, Cc3, Cc4, Cc5, Cc6, Cc7, Cc8, Cc10, Cc11, Cc12, Cc13, Cc15, Cc16, Cc17, Cc18, Cc21, Cc22, Cc23, Cc24 and Cc25;
identifying a target nucleic acid region in a gene D of the CPS/LOS biosynthesis and transport loci of C. canimorsus wherein said gene is referred to herein as a gene D and has syntenic orthologs in C. canimorsus strains Cc7 and Cc12 and not in C. canimorsus strains Cc1, Cc2, Cc3, Cc4, Cc5, Cc6, Cc8, Cc9, Cc10, Cc11, Cc13, Cc14, Cc15, Cc16, Cc17, Cc18, Cc19, Cc20, Cc21, Cc22, Cc23, Cc24 and Cc25;
identifying a target nucleic acid region in a gene of the CPS/LOS biosynthesis and transport loci of C. canimorsus, wherein said gene is referred to herein as a gene E and is unique to C. canimorsus strain Cc4; and/or
identifying a target nucleic acid region in a gene of the CPS/LOS biosynthesis and transport loci of C. canimorsus wherein said gene is referred to herein as an ABC gene and has syntenic orthologs in C. canimorsus strains Cc1, Cc2, Cc3, Cc5, Cc6, Cc8, Cc9, Cc10, Cc11, Cc13, Cc14, Cc15, Cc16, Cc17, Cc18, Cc19, Cc20, Cc21, Cc22, Cc23, Cc24 and Cc25 and not in C. canimorsus strains Cc4, Cc7 and Cc12; and
   developing an amplification primer pair configured to amplify said one or more target nucleic acid regions of an A, B, C, D, E or ABC gene.

The application also provides tools or kits of tools comprising one or more amplification primer pairs, more particularly amplification primer pairs capable of specifically amplifying target nucleic acid regions of each of said A, B, C, D, E or ABC gene, obtainable by these methods.

The determination of the presence and/or expression of a nucleic acid region in a sample can be performed in different ways, which are known in the art and include techniques such as sequencing assays, micro-arrays, PCR, RT-PCR and Northern blots. The determination of the expression level of a nucleic acid region can also be performed indirectly by measuring abundance levels of cDNAs, amplified RNAs or DNAs or quantities of DNA probes, or other molecules that are indicative of the expression level of the sequence. The information obtained by the detection method can be quantitative or can be a qualitative signal which can be translated into a quantitative measure by a user or automatically by a reader or computer system. Additionally, it can be envisaged that expression of a nucleic acid region can be detected using techniques such as antibody-binding assays, enzyme-linked immunosorbent assays (ELISAs), flow cytometry, protein assays, Western blots, nephelometry, turbidimetry, chromatography, mass spectrometry, or immunoassays

In particular embodiments, the presence of a target nucleic acid region in said gene A, gene B, gene C, gene D, gene E and/or gene ABC is determined using an amplification primer pair or probe capable of specifically detecting said target nucleic acid region(s).

In particular embodiments, expression of a target nucleic acid region in said gene A, gene B, gene C, gene D, gene E and/or gene ABC is determined by measuring RNA and/or protein levels of expression products of said target nucleic acid region(s).

In particular embodiments, the presence or expression of said target nucleic acid region in said gene A, gene B, gene C, gene D, gene E and/or gene ABC of the CPS/LOS biosynthesis and transport loci of C. canimorsus is determined by detecting the presence of capsular polysaccharides (CPS) and/or lipooligosaccharides (LOS) of C. canimorsus capsular serotype A, B, C, D and/or E in said sample.

In particular embodiments, the method for serotyping C. canimorsus in a sample, or for identifying C. canimorsus in a sample as one of serotype A, serotype B, serotype C, serotype D or serotype E, comprises the steps of, preferably being based (solely) on one or more of the following steps:
contacting said sample or at least a portion of nucleic acids isolated from said sample under conditions conducive to polymerase-based nucleic acid amplification with an amplification primer pair configured to amplify a target nucleic acid region in a gene A of the CPS/LOS biosynthesis and transport loci of C. canimorsus wherein said gene A has syntenic orthologs in C. canimorsus strain Cc1, Cc2, Cc3, Cc5, Cc10, Cc13, Cc15, Cc21, Cc22, Cc24 and Cc25, and preferably not in C. canimorsus strain Cc4, Cc6, Cc7, Cc8, Cc9, Cc11, Cc12, Cc14, Cc16, Cc17, Cc18, Cc19, Cc20 and Cc23;
an amplification primer pair configured to amplify a target nucleic acid region in a gene B of the LOS/capsule biosynthesis and transport loci of C. canimorsus wherein said gene B has syntenic orthologs in C. canimorsus strain Cc6, Cc8, Cc11, Cc16, Cc17, Cc18 and Cc23 and preferably not in C. canimorsus strain Cc1, Cc2, Cc3, Cc4, Cc5, Cc7, Cc9, Cc10, Cc12, Cc13, Cc14, Cc15, Cc19, Cc20, Cc21, Cc22, Cc24 and Cc25; an amplification primer pair configured to amplify a target nucleic acid region in a gene C of the LOS/capsule biosynthesis and transport loci of C. canimorsus wherein said gene C has syntenic orthologs in C. canimorsus strain Cc9, Cc14, Cc19 and Cc20, and preferably not in C. canimorsus strain Cc1, Cc2, Cc3, Cc4, Cc5, Cc6, Cc7, Cc8, Cc10, Cc11, Cc12, Cc13, Cc15, Cc16, Cc17, Cc18, Cc21, Cc22, Cc23, Cc24 and Cc25; an amplification primer pair configured to amplify a target nucleic acid region in a gene D of the CPS/LOS biosynthesis and transport loci of C. canimorsus wherein said gene D has syntenic orthologs in C. canimorsus strain Cc 7 and Cc12, and preferably not in C. canimorsus strains Cc1, Cc2, Cc3, Cc4, Cc5, Cc6, Cc8, Cc9, Cc10, Cc11, Cc13, Cc14, Cc15, Cc16, Cc17, Cc18, Cc19, Cc20, Cc21, Cc22, Cc23, Cc24 and Cc25; an amplification primer pair configured to amplify a target nucleic acid region in a gene E of the CPS/LOS biosynthesis and transport loci of C. canimorsus, wherein said gene E is unique to C. canimorsus strain Cc4; and/or an amplification primer pair configured to amplify a target nucleic acid region in a gene ABC of the CPS/LOS biosynthesis and transport loci of C. canimorsus wherein said gene ABC has syntenic orthologs in C. canimorsus strain Cc1, Cc2, Cc3, Cc5, Cc6, Cc8, Cc9, Cc10, Cc11, Cc13, Cc14, Cc15, Cc16, Cc17, Cc18, Cc19, Cc20, Cc21, Cc22, Cc23, Cc24 and Cc25, and preferably not in C. canimorsus strain Cc4, Cc7 and Cc12; and one or more of the following steps:
detecting the amplified target nucleic acid region in said gene A, and optionally simultaneously not detecting the amplified target nucleic acid region in said gene B, C, D and E, whereby the presence of C. canimorsus capsular serotype A in the sample is detected or whereby the C. canimorsus is identified as serotype A;
detecting the amplified target nucleic acid region in said gene B, and simultaneously not detecting the amplified target nucleic acid region in said gene A, C, D and E, whereby the presence of C. canimorsus capsular serotype B in the sample is detected or whereby the C. canimorsus is identified as serotype B;
detecting the amplified target nucleic acid region in said gene C, and simultaneously not detecting the amplified target nucleic acid region in said gene A, B, D, and E, whereby the presence of C. canimorsus capsular serotype C in the sample is detected or whereby the C. canimorsus is identified as serotype C;
detecting the amplified target nucleic acid region in said gene D, and simultaneously not detecting the amplified target nucleic acid region in said gene A, B, C, E and ABC, whereby the presence of C. canimorsus capsular serotype D in the sample is detected or whereby the C. canimorsus is identified as serotype D;
detecting the amplified target nucleic acid region in said gene E, and simultaneously not detecting the amplified target nucleic acid region in said gene A, B, C, D and ABC, whereby the presence of C. canimorsus capsular serotype E in the sample is detected or whereby the C. canimorsus is identified as serotype E; and/or
detecting the amplified target nucleic acid region in said gene ABC, and simultaneously not detecting the amplified target nucleic acid region in gene D and E, whereby the presence of C. canimorsus capsular serotype A, B and/or C in the sample is detected or whereby the C. canimorsus is identified as one of serotype A, B and/or C.

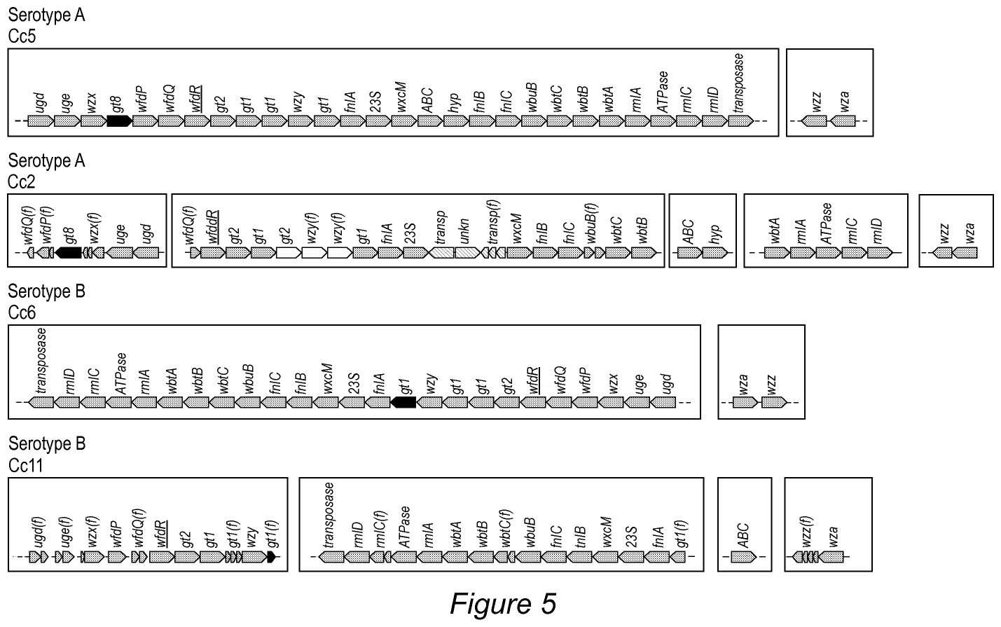

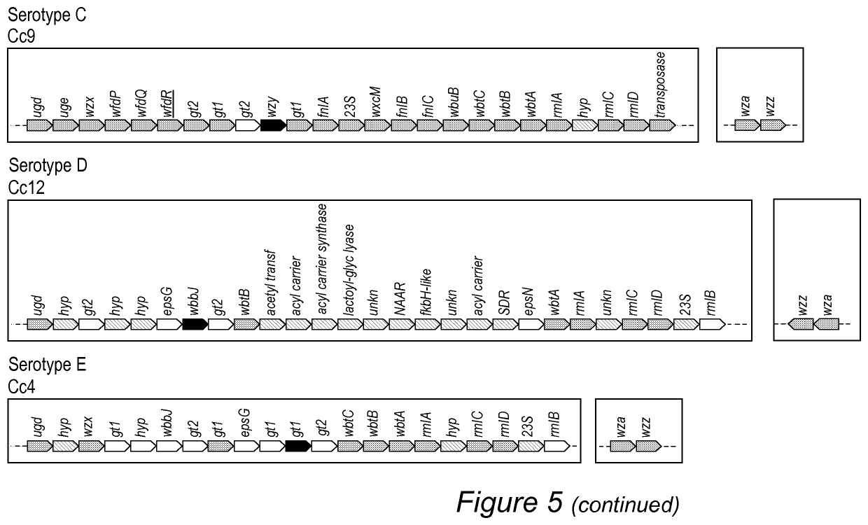

In C. canimorsus, the CPS is composed of lipopolysaccharides (LPS) O-antigen repeating units, which are composed of either homo- or heteropolymers of repeating monosaccharides joined by glycosidic linkages. The term “CPS/LOS biosynthesis and transport loci of C. canimorsus” or “O-antigen gene cluster”, as used herein, refers to a gene cluster which includes the gene encoding for the synthesis, assembly and transport of the CPS/LOS. The skilled person will understand that the size and the number of loci varies depending on the genome of a C. canimorsus strain. Examples of CPS/LOS biosynthesis and transport loci in specific C. canimorsus strains, namely C. canimorsus strain Cc2, Cc4, Cc5, Cc6, Cc9, Cc11 and Cc12, are provided in FIG. 5. For instance, in C. canimorsus 5, the CPS/LOS biosynthesis and transport loci is a 27 Kb gene cluster which includes the genes encoding for the synthesis, assembly and transport of the O-chain units, including the O-units saccharides that constitute the serotype A C. canimorsus CPS and O-antigen units, namely UDP-GalA (ugd and uge), FucNAc (fnlA, fnlB, fnlC), QuiNAc (wbtA, wbtB); homologs of genes found in the O-antigen clusters of some E. coli serotypes as wfdP, wfdQ and wfdR; genes involved in the synthesis of rahmnose (rmlA, rmlC and rmlD) that are part of the C. canimorsus 5 core; several putative glycosyltransferases; the wbuB FucNAc transferase which is crucial for the O-chain and CPS synthesis; an rfaJ homolog likely involved in the core assembly; and wzx flippase and wzy polymerase, both needed for O-chain and CPS assembly.

The terms “gene A”, “gene B”, “gene C”, “gene D”, “gene E” or “gene ABC”, as used herein, refer to any gene of the CPS/LOS biosynthesis and transport loci of C. canimorsus which can be used to detect C. canimorsus serotype A (gene A and/or gene ABC), B (gene B and/or gene ABC), C (gene C and/or gene ABC), D (gene D) or E (gene E) or to identify C. canimorsus as one of serotype A, serotype B, serotype C, serotype D or serotype E, according to the method as taught herein. The terms A, B, C, D, E or ABC are not used herein to indicate the order or position in which the genes are present in the CPS/LOS biosynthesis and transport loci of C. canimorsus.

The term “syntenic ortholog” as used herein, refers to orthologous genes whose order is conserved between two (or more) genomes. In order to determine whether two genes are under synteny, both the sequence of the two genes themselves and their flanking genes are considered. Analysis of synteny may be performed by all methods known by the skilled in the art, for example using MicroScope Microbial Genome Annotation and analysis Platform (https://www.genoscope.cns.filagc/microscope/home/index.php). The genomes of C. canimorsus strains Cc2, Cc5, Cc11 and Cc12 are available in the GenBank database (http://www.ncbi.nlm.nih.gov/). The skilled person will understand that the selection of syntenic orthologs can only be performed for those C. canimorsus strains for which the genomes are available. Putative orthologous relations between two genomes can be defined as gene couples satisfying the bi-directional best hit (BBH) criterion or a blastP alignment threshold, a minimum of 35% sequence identity on 80% of the length of the smallest protein. These relations can be subsequently used to search for conserved gene clusters, e.g. synteny groups (syntons) among several bacterial genomes. All possible kinds of chromosomal rearrangements are allowed (inversion, insertion/deletion). A gap parameter, representing the maximum number of consecutive genes which are not involved in a synteny group, can be set to five genes. Putative orthologous relations between two genomes may be determined using software programs such as BlastP and Delta Blast. For C. canimorsus strain Cc5, Ccan_23180 (ugd) to Ccan_23450 (transposase) of the CPS/LOS biosynthesis and transport loci are located in chromosome region 2483741 to 2510483 (+1) (NCBI Reference Sequence: NC_015846.1). For C. canimorsus strain Cc2, CCAN2_1920001 (wfdQ fragment) to CCAN2_1920009 (ugd) of the CPS/LOS biosynthesis and transport loci are located in chromosome region 1810154 to 1816039 (+1) (GenBank: CDOJ01000104.1); CCAN2_1430001 (wfdQ fragment) to CCAN_1430022 (wbtB) of the CPS/LOS biosynthesis and transport loci are located in chromosome region 671077 to 687045 (+1) (GenBank: CDOJ01000050.1) and CCAN2_10001 (wbtA) to CCAN2_10005 (rmlD) of the CPS/LOS biosynthesis and transport loci are located in chromosome region 9 to 4781 (+1) (GenBank: CDOJ01000001.1). For C. canimorsus strain Cc11, CCAN11_20010004 (ugd) to CCAN11_20010020 (GT1 fragment) of the CPS/LOS biosynthesis and transport loci are located in chromosome region 1116213 to 1126693 (+1) (GenBank: CDOK01000115.1) and CCAN_10011 (transposase) to CCAN11_10027 (GT1 fragment) of the CPS/LOS biosynthesis and transport loci are located in chromosome region 11146 to 24841 (+1) (GenBank: CDOK01000001.1). For C. canimorsus strain Cc6, CCAN6_1430021 (transposase) to CCAN6_1430045 (ugd) of the CPS/LOS biosynthesis and transport loci are available at http://www.urbm.be/sites/default/files/teaching/c._canimorsus_loscps_loci_sequences.pdf.

For C. canimorsus strain Cc9, CCAN9_74022 (ugd) to CCAN9_740046 (transposase) of the CPS/LOS biosynthesis and transport loci are available at http://www.urbm.be/sites/default/files/teaching/c._canimorsus_loscps_loci_sequences.pdf. For C. canimorsus strain Cc12, CCAN12_760037 (ugd) to CCAN12_760063 (rmlB) of the CPS/LOS biosynthesis and transport loci are located in chromosome region 1684022 to 17002987 (+1) (GenBank: CDOE01000074.1). For C. canimorsus strain Cc4, CCAN4_530056 (ugd) to CCAN4_530076 (rmlB) of the CPS/LOS biosynthesis and transport loci are available at. http://www.urbm.be/sites/default/files/teaching/c._canimorsus_loscps_loci_sequences.pdf.

The term “unique”, as used herein, refers to not being present in clinical C. canimorsus strains Cc1 (BCCM/LMG 11511; CCUG 17234; C. canimorsus strain P810; C. canimorsus strain SSI P810), Cc2, Cc3, Cc4, Cc5 (BCCM/LMG 28512), Cc6, Cc7, Cc8, Cc9 (BCCM/LMG 11510, CCUG 12569, CDC A3626), Cc10 (BCCM/LMG 11541, CCUG 24741, ATCC 35978, CDC C8936), Cc11 (BCCM/LMG 11551, MCCM 01373), Cc12 (ATCC 35979, CDC 7120, CCUG 53895), Cc13, Cc14, Cc15, Cc16, Cc17, Cc18, Cc19, Cc20 (CCUG 55909), Cc21 (CCUG 60839), Cc22 (CCUG 20318), Cc23 (CCUG 48899), Cc24 (CCUG 67384) and Cc25 (CCUG 66222), with the exception of the clinical C. canimorsus strain to which the gene is unique.

The term “Capnocytophaga canimorsus”, “C. canimorsus”, “CCAN” or “Cc” (formerly Centers for Disease Control group DF-2), as used herein, refers to a fastidious, slow-growing capnophilic Gram-negative bacteria that belong to the family of Flavobacteriaceae in the phylum Bacteroidetes. At present there are at least 25 C. canimorsus strains identified from human clinical isolates, more particularly C. canimorsus strains Cc1 (BCCM/LMG 11511; CCUG 17234; C. canimorsus strain P810; C. canimorsus strain SSI P810), Cc2, Cc3, Cc4, Cc5 (BCCM/LMG 28512), Cc6, Cc7, Cc8, Cc9 (BCCM/LMG 11510, CCUG 12569, CDC A3626), Cc10 (BCCM/LMG 11541, CCUG 24741, ATCC 35978, CDC C8936), Cc11 (BCCM/LMG 11551, MCCM 01373), Cc12 (ATCC 35979, CDC 7120, CCUG 53895), Cc13, Cc14, Cc15, Cc16, Cc17, Cc18, Cc19, Cc20 (CCUG 55909), Cc21 (CCUG 60839), Cc22 (CCUG 20318), Cc23 (CCUG 48899), Cc24 (CCUG 67384) and Cc25 (CCUG 66222), and at least 52 C. canimorsus strains have been identified from dog isolates, more particularly C. canimorsus strains CcD3, CcD5, CcD6, CcD10, CcD13, CcD16, CcD18, CcD20, CcD25, CcD33, CcD34, CcD35, CcD37, CcD39, CcD40, CcD43, CcD44, CcD47, CcD51, CcD52, CcD53, CcD57, CcD58, CcD63, CcD68, CcD69, CcD71, CcD73, CcD76, CcD77, CcD80, CcD81, CcD84, CcD89, CcD96, CcD101, CcD104, CcD105, CcD106, CcD113, CcD115, CcD116, CcD117, CcD118, CcD119, CcD120, CcD122, CcD124, CcD126, CcD129, CcD130 and CcD131 as previously described in Renzi F. et al. (Renzi F. et al., Only a subset of C. canimorsus strains is dangerous for humans. Emerg Microbes Infect, 2016, 5:e29). These scientific classifications are known by the skilled person. If the term “C. canimorsus” is not accompanied by the term “mutant”, the C. canimorsus as described herein is considered a “wild-type” (i.e. non-mutated form).

The term “capsular serotype”, as used herein, refers to a specific subtype of C. canimorsus strains based on specific CPS which are present on the bacterial cell surface. The capsular serotype can be serotype A, serotype B, serotype C, serotype D or serotype E. Table 1 indicates which C. canimorsus strains isolated from patients are comprised by either capsular serotype A, serotype B, serotype C, serotype D or serotype E.

TABLE 1

| | C. canimorsus strains isolated | C. canimorsus strains |
|---|---|---|
| Capsular | from patients (C. canimorsus | isolated from dog mouths |
| serotype | strain ID) | (C. canimorsus strain ID) |
| A | Cc1, Cc2, Cc3, Cc5, Cc10, Cc13, | CcD105 |
| | Cc15, Cc21, Cc22, Cc24, Cc25 | |
| B | Cc6, Cc8, Cc11, Cc16, Cc17, | CcD68 |
| | Cc18, Cc23 | |
| C | Cc9, Cc14, Cc19, Cc20 | CcD43, CcD130 |
| D | Cc7, Cc12 | CcD16, CcD89, CcD117 |
| E | Cc4 | CcD96 |

The term “sample”, as used herein, is broadly conceived and encompasses any substance or composition that may be subjected to the methods as taught herein. Useful samples may in particular encompass samples from biological specimens obtained from subjects, and microorganisms (particularly bacteria) isolated and/or propagated (e.g., cultured in vitro as known per se) from such biological specimens. Preferably, a biological specimen is readily obtainable by non-invasive or minimally invasive methods, allowing to remove or isolate said specimen from the subject. Representative biological specimens and samples that can be used in practicing the methods as taught herein include without limitation oral swabs, saliva, nasal swabs, throat swabs, faeces, faecal swabs, perineum swabs dermal swabs, blood (including blood culture), sputum, bronchio-alveolar lavage, bronchial aspirates, lung tissue, and urine. Preferably, the sample is a direct sample collected from a subject or at least a portion of nucleic acids isolated from the sample is directly subjected to the methods as taught herein, without prior isolation and/or propagation (culture) of microorganisms (particularly bacteria) from the sample. The use of direct samples as facilitated by methods embodying the principles of the present invention allows inter alia to simplify the analysis and reduce turnaround times. Preferably, if the subject is a canine or feline, the sample is an oral swab or saliva.

As samples from biological specimens obtained from subject might also comprise other microorganisms than C. canimorsus bacteria, it might be of interest to isolate and culture the bacteria of interest from the sample.

In particular embodiments, the methods as described herein comprise a step of isolating and cultivating C. canimorsus bacteria from the sample, and optionally repeating the methods as described herein on the cell colonies obtained by the cultivation step.

The term “subject”, as used herein, typically and preferably denotes humans, but may also encompass reference to non-human animals, preferably warm-blooded animals, more preferably vertebrates, even more preferably mammals, such as, e.g., non-human primates, rodents, canines (e.g. dog), felines, equines, ovines, porcines, and the like. Particularly suitable may be subjects known to harbour or suspected of harbouring C. canimorsus, preferably potentially pathogenic C. canimorsus. The presence of C. canimorsus in a sample may be identified by methods known by the skilled person, such as PCR specific detection of 16S rRNA gene as reported by Suzuki et al. (Suzuki et al., 2010, Vet Microbiol). By investigating samples from subjects, the present methods offer comprehensive information about the presence of potentially pathogenic C. canimorsus in the subjects.

Samples for screening in the methods as taught herein, such as direct samples as defined herein, may be generally only minimally processed prior to being subjected to the methods. For example, the samples may be treated to lyse microorganisms such as bacteria present therein, using any acceptable method known in the art, e.g., chemical methods employing for example detergents and/or chaotropic salts and/or physical methods employing for example heat and/or mechanical cell disintegration, and cellular debris may be suitably removed, for example by centrifugation, sedimentation or filtration, whereby the supernatant is retained for analysis.

If the sample is saliva from a dog or a cat obtained via an oral swab, the sample is preferably heated at a temperature of at least 95° C. and at most 100° C., such as 96° C., 97° C., 98° C. or 99° C., preferably 98° C. for a period of at least 10 min, preferably 15 min, before using it as a template for the polymerase-based nucleic acid amplification.

In particular embodiments, the sample is subjected to an immuno-purification step prior to contacting said sample or at least a portion of nucleic acids isolated from said sample under conditions conducive to polymerase-based nucleic acid amplification with said amplification primer pair; wherein said immuno-purification step is performed employing one or more polyclonal antibodies recognizing capsular polysaccharides (CPS) and/or lipooligosaccharides (LOS) of wild-type C. canimorsus bacteria of one or more but not all capsular serotypes as described elsewhere herein. The skilled person will understand that the immunopurified sample will only comprise bacteria binding to the polyclonal antibody recognizing capsular polysaccharides (CPS) and/or lipooligosaccharides (LOS) of wild-type C. canimorsus bacteria, more particularly of one or more but not all capsular serotypes as described elsewhere herein. The immuno-purification step may be performed by any known methods for immuno-purification of bacteria, such as immuno-adsorption, immuno-precipitation or fluorescence-activated cell sorting.

In certain embodiments, nucleic acids may be isolated from samples, such as direct samples, and the methods may employ at least a portion of so-isolated nucleic acids. The term “isolated” with reference to a particular component (e.g., a nucleic acid) generally denotes that such component exists in separation from—for example, has been separated from or prepared and/or maintained in separation from—one or more other components of its natural environment. For instance, an isolated bacterial nucleic acid may exist in separation from the bacterium where it naturally occurs. The term “isolated”, as used herein, may preferably also encompass the qualifier “purified”. By means of example, the term “purified” with reference to a substance (e.g., a nucleic acid) does not require absolute purity. Instead, it denotes that such substance is in a discrete environment in which its abundance (conveniently expressed in terms of mass or weight or concentration) relative to other relevant substances is greater than in a sample. A discrete environment denotes a single medium, such as for example a single solution, gel, precipitate, lyophilisate, etc. Purified nucleic acids may be obtained by methods routinely known in the art, e.g., nucleic acids present in the supernatant as mentioned above may be precipitated, e.g., by ethanol precipitation, pelleted, washed, and re-suspended in an appropriate buffer. Purity and quantity of nucleic acids may be determined by measuring absorbance A₂₆₀/A₂₈₀.

The phrase “polymerase-based nucleic acid amplification”, as used herein, generally encompasses any in vitro process for increasing the number of copies of a target nucleic acid region within a nucleic acid molecule, preferably within a DNA molecule, by the action of a nucleic acid polymerase, e.g., DNA polymerase. The process may encompass both linear and exponential amplification, and particularly preferably refers to exponential amplification. The process may particularly preferably refer to polymerase chain reaction (PCR). In PCR, target nucleic acid region within a nucleic acid molecule, especially within a DNA molecule, is amplified using thermostable DNA polymerase(s) and at least two amplification primers, one complementary to the (+)-strand at one end of the target sequence to be amplified and the other complementary to the (−)-strand at the other end of the target sequence. A reference to PCR, as used herein, encompasses modifications of the prototypic PCR, such as, e.g., high-fidelity PCR, hot-start PCR, touch-down PCR, nested PCR, multiplex PCR, quantitative PCR, quantitative real-time PCR, long-range PCR, RT-PCR, etc. (see, e.g., PCR Protocols: A Guide to Methods and Applications, eds. Innis et al., Academic Press, San Diego, 1990). PCR is well known in the art and can be directly applied or adapted for use with the primers and primer pairs as described herein, and methods as described herein. Methods for setting up a PCR reaction are well known to those skilled in the art. The reaction mixture minimally comprises template nucleic acid (except in the case of a negative control) and oligonucleotide primers in combination with suitable buffers, salts, and the like, and an appropriate concentration of a nucleic acid polymerase.

The methods as taught herein comprise contacting a sample or at least a portion of nucleic acids isolated from the sample under conditions conducive to polymerase-based nucleic acid amplification with primer pairs configured to amplify genes of the CPS/LOS biosynthesis and transport loci (including A4galT GT, GT1, wzy, wfdR and/or wbbJ) under said conditions as taught herein.

The reference to “conditions conducive to polymerase-based nucleic acid amplification” means that the conditions, such as in particular the composition of the amplification reaction and the physical conditions to which the amplification reaction is subjected (in particular temperature cycling conditions) are sufficient to effect amplification of target nucleic acid regions in the genes of the CPS/LOS biosynthesis and transport loci (i.e. A4galT GT, GT1, wzy, wfdR and/or wbbJ by the respective primer pairs as taught herein.

By “nucleic acid” is meant oligomers and polymers of any length composed essentially of nucleotides, e.g., deoxyribonucleotides and/or ribonucleotides. Nucleic acids can comprise purine and/or pyrimidine bases and/or other natural (e.g., xanthine, inosine, hypoxanthine), chemically or biochemically modified (e.g., methylated), non-natural, or derivatised nucleotide bases. The backbone of nucleic acids can comprise sugars and phosphate groups, as can typically be found in RNA or DNA, and/or one or more modified or substituted sugars and/or one or more modified or substituted phosphate groups. Modifications of phosphate groups or sugars may be introduced to improve stability, resistance to enzymatic degradation, or some other useful property. A “nucleic acid” can be for example double-stranded, partly double stranded, or single-stranded. Where single-stranded, the nucleic acid can be the sense strand or the antisense strand. In addition, nucleic acid can be circular or linear. The term “nucleic acid”, as used herein, preferably encompasses DNA and RNA, specifically including RNA, genomic RNA, cDNA, DNA, provirus, pre-mRNA and mRNA.

As used herein, the term “primer” refers to a single-stranded oligonucleotide sequence, more preferably to a DNA oligonucleotide, which is (or part of which is) complementary or sufficiently complementary to a sequence comprised in a nucleic acid to be amplified by polymerase-based amplification process, e.g., PCR, such that the primer can hybridise (anneal) with said sequence and can act as a point of initiation of synthesis of a primer extension product in the presence of nucleotides and a nucleic acid polymerase, e.g., DNA polymerase. The length and sequence of a primer are determined to be suitable for initiating the synthesis of an extension product. A typical primer may thus be at least 10 nucleotides in length, e.g., at least 11, at least 12, at least 13 or at least 14 nucleotides in length, preferably at least 15 nucleotides in length, e.g., at least 16, at least 17, at least 18 or at least 19 nucleotides in length, more preferably at least 20 nucleotides in length. Further preferred primers are between about 10 and about 40 nucleotides in length, more preferably between about 15 and about 30 nucleotides in length, most preferably between about 18 and about 26 nucleotides long. Primers can be designed using, for example, a computer program such as primer designing tools. Non-limiting examples are Primer3 Plus (Bioinformatics), Primer3 (Simgene). The skilled person will understand that the size amplification product, melting temperatures of a primer pair and the length of the primer are important features when designing oligonucleotides to be used as amplification primers.

In certain embodiments, primers as taught herein may be defined as configured to hybridise (anneal) within certain recited nucleic acid sequences. In this context, the phrase “hybridise within a nucleic acid” or “hybridise within a nucleic acid sequence” is intended to mean that the primer may anneal to the whole of the recited nucleic acid sequence, or only to a portion of the recited nucleic acid sequence, but does not anneal to sequences adjacent to but outside of the recited nucleic acid sequence.

The terms “hybridisation” and “hybridise” refer to a process by which a nucleic acid strand anneals with complementary or sufficiently complementary sequence(s) comprised in the same or another nucleic acid strand through base pairing, particularly Watson-Crick base pairing. The terms “complementary” or “complementarity”, as used herein with reference to nucleic acids, refer to the normal binding of single-stranded nucleic acids under permissive salt (ionic strength) and temperature conditions by base pairing, particularly Watson-Crick base pairing. By means of example, complementary Watson-Crick base pairing occurs between the bases A and T, A and U or G and C. For example, the sequence 5′-A-G-T-3′ is complementary to sequence 5′-A-C-T-3′.

A primer said to hybridise within a given nucleic acid may in certain embodiments be wholly complementary to the sequence or portion thereof with which it anneals. In other embodiments, the primer may be partly but not wholly complementary to said sequence. For example, the primer may display one or more, typically only one or two, substitutions, deletions or additions vis-à-vis a primer that would be wholly complementary to said sequence. Hence, such primer, while not being wholly complementary, is sufficiently complementary to act as a point of initiation of synthesis of a primer extension product in the polymerase-based amplification reaction.

Hybridisation and the strength of hybridisation (i.e., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components (e.g., the presence or absence of polyethylene glycol), the molarity of the hybridizing strands and the G:C content of the polynucleotide strands.

A primer as taught herein thus comprises an oligonucleotide sequence which effects the hybridisation (annealing) of the primer with its respective target nucleic acid. In certain embodiments, a primer does not contain any further oligonucleotide sequence(s). In certain other embodiments, a primer may contain—besides the oligonucleotide sequence which effects the hybridisation of the primer with its target nucleic acid—additional oligonucleotide sequence(s) serving other useful purpose(s). For example but without limitation, such additional oligonucleotide sequence(s) may provide primer-binding sequences allowing for subsequent amplification or sequencing of the initial amplification product, or may provide probe-binding sequences allowing for subsequent hybridisation of the initial amplification product with probes or capture probes (e.g., on (micro)arrays), or may provide cloning adaptor sequences facilitating cloning of the initial amplification product to nucleic acid constructs, e.g., by restriction enzyme- or recombination-mediated cloning, or may provide linker sequences allowing to couple a primer with another moiety or moieties, e.g., label(s), etc; various options are available to a skilled reader. Such additional oligonucleotide sequence(s) may be suitably arranged at the 5′ terminus of the primer, such that the primer extension reaction from the 3′ end of the primer is not altered.

In certain embodiments, the primer pairs may be designed such that the melting temperature (Tm) of one primer in the pair is within 2° C. of the Tm of the other primer in the pair. In further embodiments, the primers in the set may be designed such that the melting temperature (Tm) of one primer in the set is within 10° C. of the Tm of any other primer in the set. Tm similarity will help to ensure that different target sequences are amplified with comparable efficiencies at given conditions (e.g., PCR cycling conditions).

As used herein, “Tm” and “melting temperature” are interchangeable terms which are the temperature at which 50% of a population of double-stranded polynucleotide molecules becomes dissociated into single strands. The equation for calculating the Tm of polynucleotides is well known in the art. For example, the Tm may be calculated by the following equation: Tm=69.3+0.41×(G+C) %−650/L, wherein L is the length of the probe in nucleotides. The Tm of a hybrid polynucleotide may also be estimated using a formula adopted from hybridisation assays in 1 M salt, and commonly used for calculating Tm for PCR primers: [(number of A+T)×2° C.+(number of G+C)×4° C.]. Other more sophisticated computations exist in the art, which take structural as well as sequence characteristics into account for the calculation of Tm. A calculated Tm is merely an estimate; the optimum temperature is commonly determined empirically.

The term “primer pair” or “amplification primer pair” refers to a combination of two primers which are suited for amplification of a target nucleic acid region (amplicon) from within a nucleic acid of interest by a polymerase-based amplification process, e.g., PCR. More particularly, such a primer pair may comprise a forward primer (or 5′ primer) which anneals to the template (−) strand of dsDNA and a reverse primer (or 3′ primer) which anneals to the template (+) strand of dsDNA. The ability to amplify an amplicon from within the nucleic acid of interest using a primer pair designed to specifically hybridise within the nucleic acid indicates the presence (and optionally quantity) of the nucleic acid in the polymerase-based amplification reaction. The skilled person will understand that a primer pair requires similar melting temperatures since annealing in a PCR occurs for the forward and the reverse primer simultaneously.

The term “oligonucleotide”, as used herein, refers to a nucleic acid (including nucleic acid analogues and mimetics) oligomer or polymer as defined herein. Preferably, an oligonucleotide is (substantially) single-stranded. Oligonucleotides as intended herein may be preferably between about 10 and about 100 nucleoside units (i.e., nucleotides or nucleotide analogues) in length, preferably between about 15 and about 50, more preferably between about 15 and about 40, also preferably between about 20 and about 30.

As used herein, the term “probe” refers to an oligonucleotide, more preferably to a DNA oligonucleotide, which (or part of which) is complementary or sufficiently complementary as defined herein to a sequence comprised in a nucleic acid to be detected by the probe, such that the probe can hybridise (anneal) with said sequence. In particular, probes as intended herewith can hybridise (anneal) with a primer extension product produced by the polymerase-based amplification.

In certain embodiments, probes as taught herein may be defined as configured to hybridise (anneal) specifically within certain recited nucleic acid sequences. In this context, the phrase “hybridise within a nucleic acid” or “hybridise within a nucleic acid sequence” is intended to mean that the probe may anneal to the whole of the recited nucleic acid sequence, or only to a portion of the recited nucleic acid sequence, but does not anneal to sequences adjacent to but outside of the recited nucleic acid sequence.

Probes may be ideally less than or equal to about 50 nucleotides in length, for example less than or equal to about 40, about 30, about 20, or less than about 10 nucleotides in length, e.g., between 10 and 30 or between 15 and 25 nucleotides in length.

A probe as taught herein thus comprises an oligonucleotide sequence which effects the hybridisation (annealing) of the probe with a sequence comprised in a nucleic acid to be detected by the probe. In certain embodiments, a probe does not contain any further oligonucleotide sequence(s). In certain other embodiments, a probe may contain—besides the oligonucleotide sequence which effects the hybridisation of the probe with a sequence comprised in a nucleic acid to be detected by the probe—additional oligonucleotide sequence(s) serving other useful purpose(s). For example but without limitation, such additional oligonucleotide sequence(s) may provide linker sequences allowing to couple a probe with another moiety or moieties, e.g., label(s), or may provide sequences ensuring a certain conformation of a probe, etc.; various options are available to a skilled reader.

By means of example and not limitation, when a probe forms a molecular beacon as known in the art, mutually complementary oligonucleotide extensions are provided at the 5′ and 3′ ends of the probe, one of the oligonucleotide extensions linked to a fluorophore and the other one to a quencher capable of quenching the fluorescent emission of the fluorophore. When the probe is not annealed to the nucleic acid to be detected, the mutually complementary oligonucleotide extensions will form a hairpin structure, whereby the quencher is brought into proximity of the fluorophore and quenches the fluorophore's signal. Conversely, when the probe is annealed to the nucleic acid to be detected, the hairpin structure cannot formed, the quencher is not in proximity of the fluorophore and does not quench the fluorophore's signal, which signal is therefore detectable.

Furthermore, the use of probes based on the TAQMAN® probe technology is also envisaged, as explained elsewhere in this application. Such latter probes need not contain oligonucleotide stretches responsible for stem structure formation, in contrast to molecular beacon probes. Such variations on probe technology and design are available to the skilled reader.

In particular embodiments, oligonucleotide probes designed to hybridise specifically with the target nucleic acid regions can be used to detect the target nucleic acid region. Such probes may also be used in conjunction with amplification primers to facilitate detection of the amplified target nucleic acid regions.

In particular embodiments, the oligonucleotide probe configured to hybridise with a target nucleic acid region in A4galT GT is configured to hybridize within a nucleic acid sequence which is at least 90%, preferably at least 95%, identical to SEQ ID NO: 25; the oligonucleotide probe configured to hybridise with a target nucleic acid region in the first GT1 is configured to hybridize within a nucleic acid sequence which is at least 90%, preferably at least 95%, identical to SEQ ID NO: 26 or 50, the oligonucleotide probe which is configured to hybridise with a target nucleic acid region in wzy is configured to hybridize within a nucleic acid sequence which is at least 90%, preferably at least 95%, identical to SEQ ID NO: 27, the oligonucleotide probe which is configured to hybridise with a target nucleic acid region in wbbJ is configured to hybridize within a nucleic acid sequence which is at least 90%, preferably at least 95%, identical to SEQ ID NO: 28, the oligonucleotide probe which is configured to hybridise with a target nucleic acid region in the second GT1 is configured to hybridize within a nucleic acid sequence which is at least 90%, preferably at least 95%, identical to SEQ ID NO: 29, and/or the oligonucleotide probe which is configured to hybridise with a target nucleic acid region in wfdR is configured to hybridize within a nucleic acid sequence which is at least 90%, preferably at least 95%, identical to SEQ ID NO: 30, 31 or 32.

In particular embodiments, the polymerase-based nucleic acid amplification may be polymerase chain reaction (PCR). In certain embodiments, the polymerase-based nucleic acid amplification may be quantitative, i.e., it provides information about the quantity of the amplification products and by extension about the quantity of the templates (i.e., said gene A, gene B, gene C, gene D, gene E or gene ABC of the CPS/LOS biosynthesis and transport loci of C. canimorsus). In particularly preferred embodiments, the polymerase-based nucleic acid amplification may be real-time quantitative amplification, more preferably real-time quantitative PCR. Real-time quantitative PCR is commonly known in the art as simply “quantitative PCR” (qPCR, QPCR) or as real-time qPCR, real-time QPCR, RT-qPCR or RT-QPCR. Real-time quantitative PCR may be preferred under some circumstances, because it provides not only a quantitative measurement, but also reduced time and contamination.

The step of contacting the sample or at least a portion of nucleic acids isolated from said sample under conditions conducive to polymerase-based nucleic acid amplification with an amplification primer pair configured to amplify a target nucleic acid region in a gene A of the CPS/LOS biosynthesis and transport loci of C. canimorsus as taught herein, an amplification primer pair configured to amplify a target nucleic acid region in a gene B of the CPS/LOS biosynthesis and transport loci of C. canimorsus as taught herein, an amplification primer pair configured to amplify a target nucleic acid region in a gene C of the CPS/LOS biosynthesis and transport loci of C. canimorsus as taught herein, an amplification primer pair configured to amplify a target nucleic acid region in a gene D of the CPS/LOS biosynthesis and transport loci of C. canimorsus as taught herein, an amplification primer pair configured to amplify a gene E of the CPS/LOS biosynthesis and transport loci of C. canimorsus as taught herein and/or an amplification primer pair configured to amplify a target nucleic acid region in a gene ABC of the CPS/LOS biosynthesis and transport loci of C. canimorsus as taught herein can be accomplished by independent polymerase-based nucleic acid amplification reactions (e.g. PCR reactions), meaning that each polymerase-based nucleic acid amplification reaction comprises only one of the amplification primer pairs as described herein, or alternatively by one multiplexed polymerase-based nucleic acid amplification reaction (e.g. PCR reaction) comprising one or more, preferably five, of the amplification primer pairs as described herein, preferably wherein said amplification primers are labeled.

In particularly preferred embodiments, the polymerase-based nucleic acid amplification as taught herein, such as PCR or QPCR, may multiplexed, such that at least two, at least three, at least four, or at least five of the target nucleic acid regions are amplified in the same polymerase-based nucleic acid amplification reaction.

Accordingly, primer pairs as taught herein to amplify at least two, at least three, at least four, or at least five of the target nucleic acid regions will be included in the same polymerase-based nucleic acid amplification reaction. By reducing the number of reactions to set-up, multiplexing advantageously reduces the amount of sample needed, cuts down the processing and machine space required, reduces variability between reactions, etc.

Preferably, the multiplexed polymerase-based nucleic acid amplification as taught herein amplifies the target nucleic acid region in said gene A, B, C, D and E or said gene ABC, D and Eas taught herein in the same polymerase-based nucleic acid amplification reaction. Numerous different PCR or QPCR protocols are known in the art and can be directly applied or adapted for use with the primers and primer pairs as described herein, and methods as described herein. Generally, in PCR, a target polynucleotide sequence is amplified by reaction with a pair of oligonucleotide primers. The primers hybridise to complementary regions of a target nucleic acid and a DNA polymerase extends the primers to amplify the target sequence, generating an amplification product. The amplification cycle is repeated to increase the concentration of the amplification product. The reaction can be performed in any thermocycler commonly used for PCR.

As used herein, “quantitative PCR” (or “real-time QPCR”) refers to the direct monitoring of the progress of a PCR amplification as it is occurring without the need for repeated sampling of the reaction products. In QPCR, the reaction products may be monitored via a signalling mechanism (e.g., fluorescence) as they are generated and are tracked after the signal rises above a background level but before the reaction reaches a plateau. The number of cycles required to achieve a detectable or “threshold” level of fluorescence (“cycle threshold”, “CT”) varies directly with the concentration of amplifiable targets at the beginning of the PCR process, enabling a measure of signal intensity to provide a measure of the amount of target nucleic acid in a sample in real time.

In preferred embodiments, labelled probes may be used to detect the extension product generated by PCR amplification. Any probe format utilising labelled probes as taught herein may be used, e.g., such as SCORPIONS™ probes, sunrise probes, TAQMAN® probes, or molecular beacon probes, as is known in the art.

The PCR or QPCR reaction may contain various controls. Such controls should include a “no template” negative control, in which primers, buffer, enzyme(s) and other necessary reagents (e.g., magnesium chloride, nucleotides) are cycled in the absence of added test sample. A positive control including a known target nucleic acid should also be run in parallel. Both positive control and negative control may be included in the amplification reaction. A single reaction may contain either a positive control, a negative control, or a sample template. In addition to “no template” controls, negative controls can also include amplification reactions with non-specific target nucleic acid included in the reaction, or can be samples prepared using any or all steps of the sample preparation (from nucleic acid extraction to amplification preparation) without the addition of a test sample (e.g., each step uses either no test sample or a sample known to be free of C. canimorsus).

Where the starting material for the PCR reaction is RNA, complementary DNA (cDNA) is made from RNA via reverse transcription. A PCR used to amplify RNA products is referred to as reverse transcriptase PCR or “RT-PCR.” The term “RT-PCR”, as used herein, refers to reverse transcription-polymerase chain reaction. RT-PCR is a technique that is used to amplify cDNA reversely transcribed from a RNA template, and involves the synthesis of cDNA from RNA using reverse transcriptase; and amplification of a specific site of cDNA.

In certain embodiments, the primers used in the methods as taught herein may comprise detectable labels. Preferably, the detectable labels may allow for individual detection and monitoring of each of the amplified target nucleic acid regions. Preferably the detectable labels may comprise distinct fluorophores having distinct excitation and/or emission characteristics, such that each of the amplified target nucleic acid regions can be individually detected by detecting the corresponding fluorophore.

Present inventors have shown that from all genes of the CPS/LOS biosynthesis and transport loci in C. canimorsus, a A4galT-like glycosyltransferase gene (i.e. A4galT GT), two different putative family 1 glycosyltransferase genes (i.e. GT1), a putative O-antigen polymerase (i.e. wzy), a putative lipopolysaccharide biosynthesis O-acetyl transferase (i.e. wbbJ) and/or a putative glycosyltransferase (i.e. wfdR) are particularly suitable to differentiate between capsular serotypes A, B, C, D and E.

In view hereof, in particular embodiments of the method for serotyping C. canimorsus in a sample as taught herein, said gene A of the CPS/LOS biosynthesis and transport loci of C. canimorsus is A4galT GT, said gene B of the CPS/LOS biosynthesis and transport loci of C. canimorsus is a first GT1, said gene C of the CPS/LOS biosynthesis and transport loci of C. canimorsus is wzy, said gene D of the CPS/LOS biosynthesis and transport loci of C. canimorsus is wbbJ; said gene E of the CPS/LOS biosynthesis and transport loci of C. canimorsus is a second GT1 and said gene ABC of the CPS/LOS biosynthesis and transport loci of C. canimorsus is wfdR.

In particular embodiments, the method for serotyping C. canimorsus or identifying C. canimorsus as one of serotype A, serotype B, serotype C, serotype D or serotype E in a sample as taught herein, comprises the step of
contacting said sample or at least a portion of nucleic acids isolated from said sample under conditions conducive to polymerase-based nucleic acid amplification with an amplification primer pair configured to amplify a target nucleic acid region in C. canimorsus A4galT GT, wherein the coding nucleic acid sequence of said gene is at least 90%, preferably at least 95%, more preferably 100% identical to SEQ ID NO: 1, an amplification primer pair configured to amplify a target nucleic acid region in a first C. canimorsus GT1, wherein the coding nucleic acid sequence of said gene is at least 90%, preferably at least 95%, more preferably 100% identical to SEQ ID NO: 2 and/or 49; an amplification primer pair configured to amplify a target nucleic acid region in C. canimorsus wzy, wherein the coding nucleic acid sequence of said gene is at least 90%, preferably at least 95%, more preferably 100% identical to SEQ ID NO: 3, an amplification primer pair configured to amplify a target nucleic acid region in C. canimorsus wbbJ; wherein the coding nucleic acid sequence of said gene is at least 90%, preferably at least 95%, more preferably 100% identical to SEQ ID NO: 4, an amplification primer pair configured to amplify a second C. canimorsus GT1, wherein the coding nucleic acid sequence of said gene is at least 90%, preferably at least 95%, identical to SEQ ID NO: 5 and/or an amplification primer pair configured to amplify a target nucleic acid region in C. canimorsus wfdR, wherein the coding nucleic acid sequence of said gene is at least 90%, preferably at least 95%, more preferably 100% identical to SEQ ID NO: 6, 7 and/or 8; and one or more of the following steps:
detecting the amplified target nucleic acid region in A4galT GT, whereby the presence of C. canimorsus capsular serotype A in the sample is detected or whereby C. canimorsus is identified as serotype A;
detecting the amplified target nucleic acid region in the first GT1 and simultaneously not detecting the amplified target nucleic acid region in A4galT GT, whereby the presence of C. canimorsus capsular serotype B in the sample is detected or whereby C. canimorsus is identified as serotype B;
detecting the amplified target nucleic acid region in wzy and simultaneously not detecting the amplified target nucleic acid region in the A4galT GT, whereby the presence of C. canimorsus capsular serotype C in the sample is detected or whereby C. canimorsus is identified as serotype C;
detecting the amplified target nucleic acid region in wbbJ, whereby the presence of C. canimorsus capsular serotype D in the sample is detected or whereby C. canimorsus is identified as serotype D;
detecting the amplified target nucleic acid region in the second GT1, whereby the presence of C. canimorsus capsular serotype E in the sample is detected or whereby C. canimorsus is identified as serotype E; and/or
detecting the amplified target nucleic acid region in wfdR whereby the presence of serotype A, B or C in the sample is detected or whereby C. canimorsus is identified as one of serotype A, B or C.

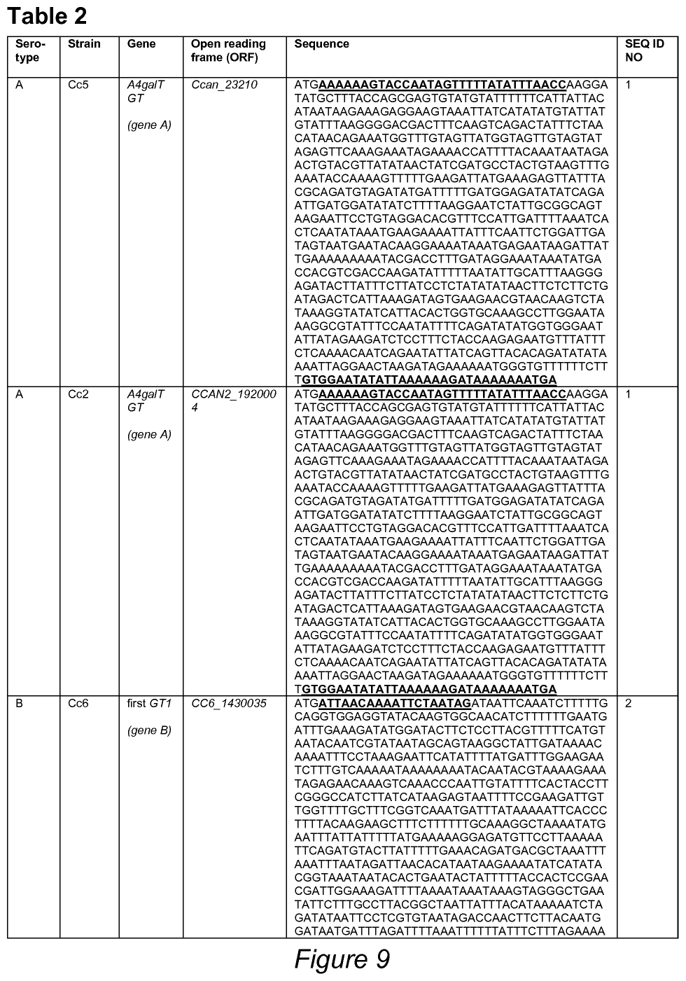

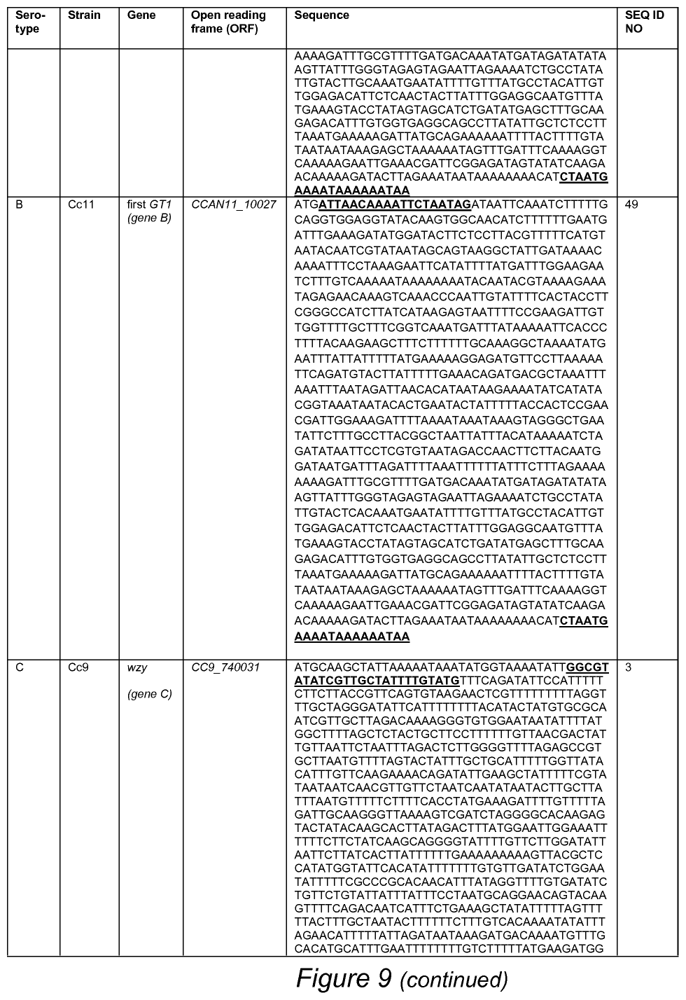

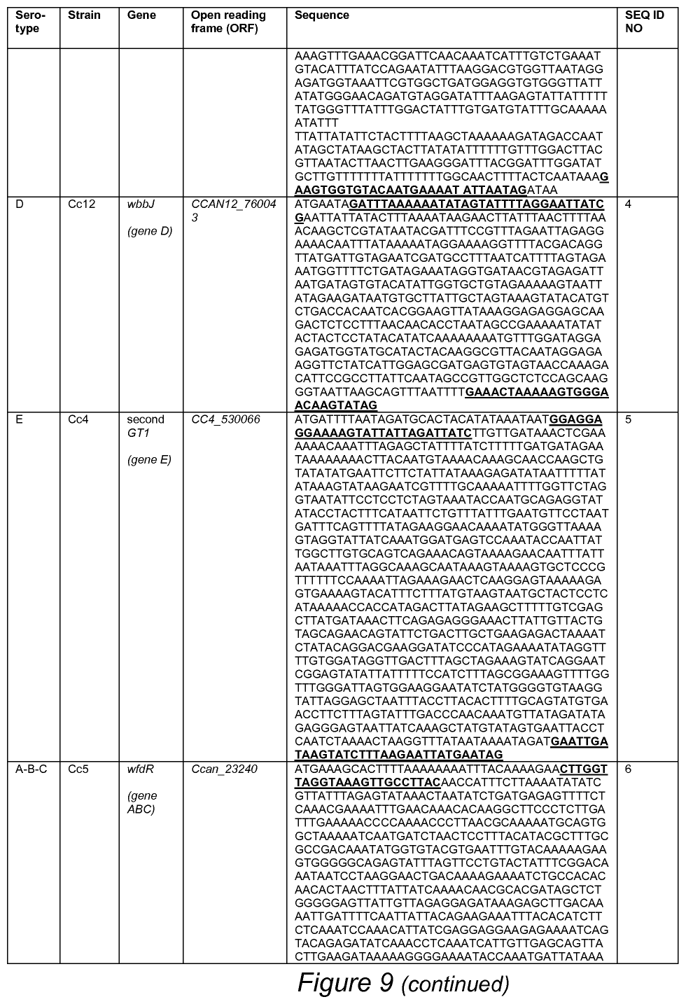

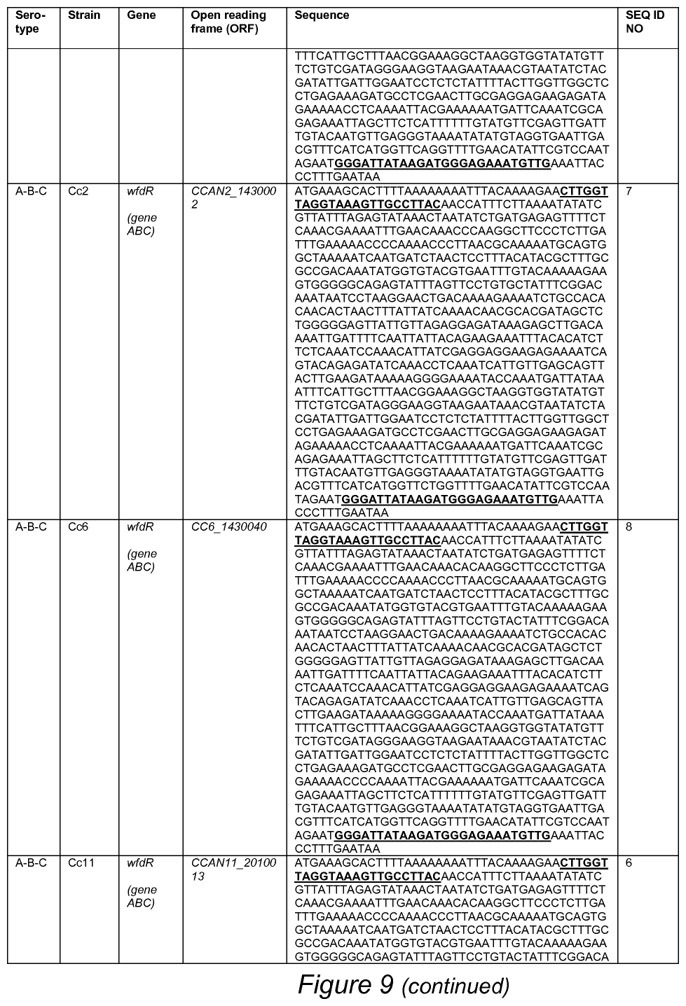

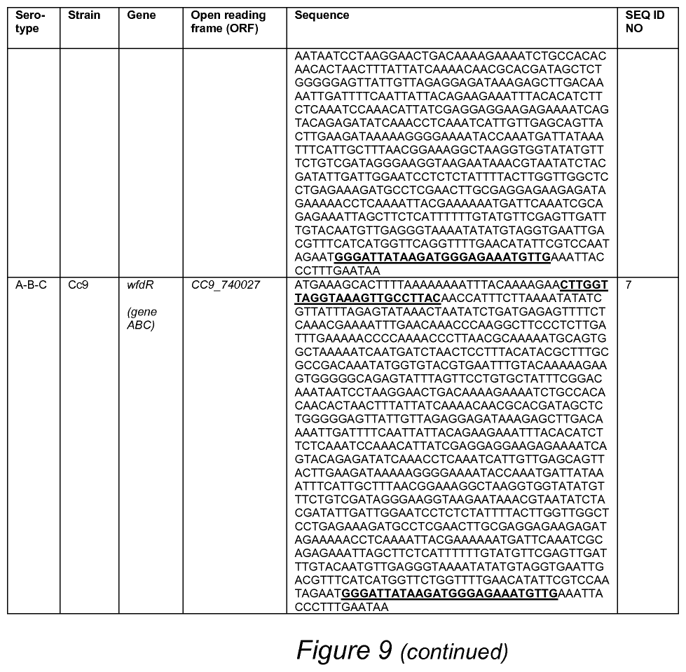

Nucleotide sequences of genes encoding C. canimorsus A4galT GT, GT1 (i.e. two different GT1 genes), wzy, wfdR and wbbJ as taught herein are provided in Table 2 (FIG. 9).

The term “A4GalT-like glycosyltransferase gene” or “A4galT GT”, as used herein, refers to a gene located in the CPS/LOS biosynthesis and transport loci that encodes a A4galT-like glycosyltransferase in C. canimorsus. This gene is also known as Ccan_23210 in C. canimorsus 5 (SEQ ID NO: 1) and as CCAN2_1920004 in C. canimorsus 2 (SEQ ID NO: 1). The term “family 1 glycosyltransferase gene” or “GT1”, as used herein, refers to a gene located in the CPS/LOS biosynthesis and transport loci that encodes a putative family 1 glycosyltransferase in C. canimorsus. The CPS/LOS biosynthesis and transport loci in C. canimorsus comprise several GT1 genes. A “first GT1”, as used herein, is also known as CC6_1430035 in C. canimorsus 6 (SEQ ID NO: 2) and as CCAN11_10027 in C. canimorsus 11 (SEQ ID NO: 49). A “second GT1”, as used herein, is also known as CC4_530066 in C. canimorsus 4 (SEQ ID NO: 5). The first GT1 and the second GT1 are not orthologs and both catalyze different reactions.

The term “wzy”, as used herein, refers to a gene located in the CPS/LOS biosynthesis and transport loci that encodes a putative O-antigen polymerase in C. canimorsus. This gene is also known as CC9_740031 in C. canimorsus 9 (SEQ ID NO: 3).

The term “wbbJ”, as used herein, refers to a gene located in the CPS/LOS biosynthesis and transport loci that encodes a putative lipopolysaccharide biosynthesis O-acetyl transferase in C. canimorsus. The gene is also known as CCAN12_760043 in C. canimorsus 12 (SEQ ID NO: 4).

The term “wfdR”, as used herein, refers to a gene located in the CPS/LOS biosynthesis and transport loci that encodes a putative glycosyltransferase in C. canimorsus. The gene is also known as Ccan_23290 in C. canimorsus 5 (SEQ ID NO: 6), as CCAN2_201002 in C. canimorsus 2 (SEQ ID NO:7), as CC6_1430035 in C. canimorsus 6 (SEQ ID NO: 8), as CCAN11_201002 in C. canimorsus 11 (SEQ ID NO: 6) and as CC9_740032 in C. canimorsus 9 (SEQ ID NO: 7).

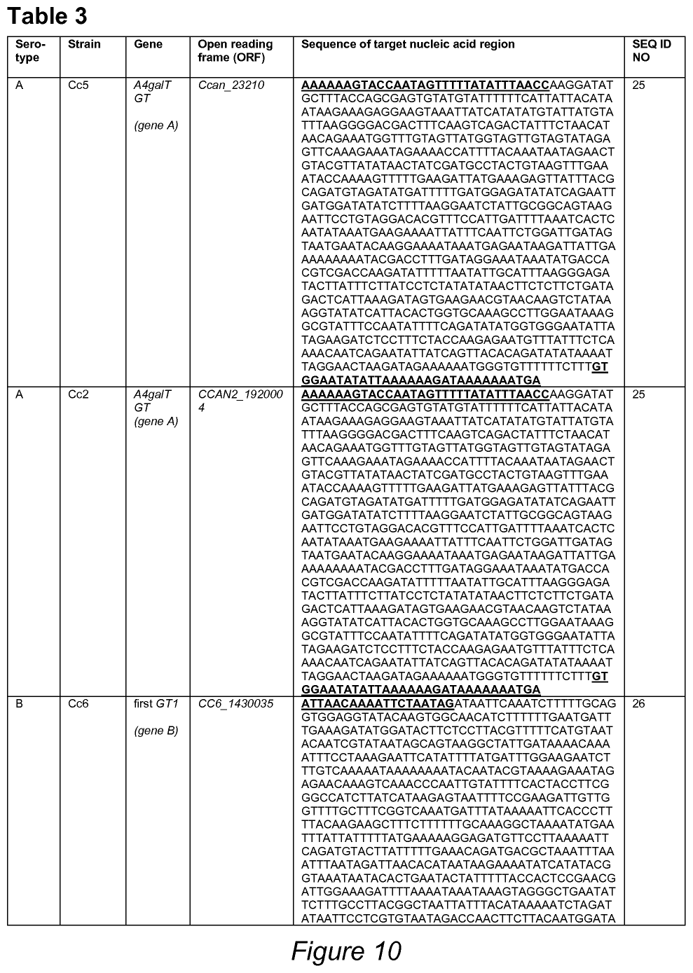

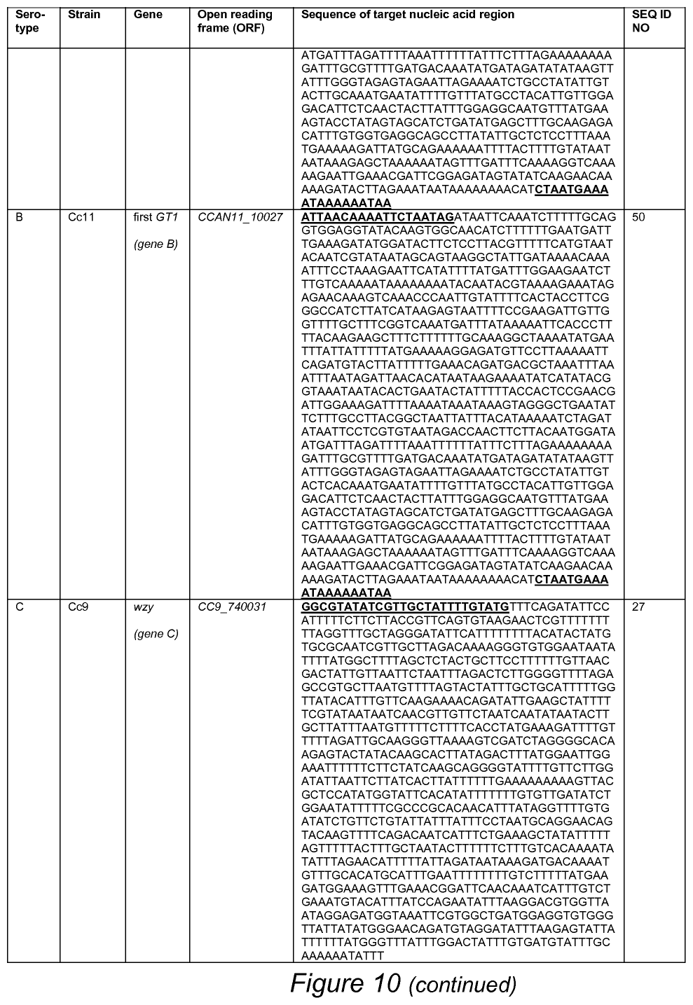

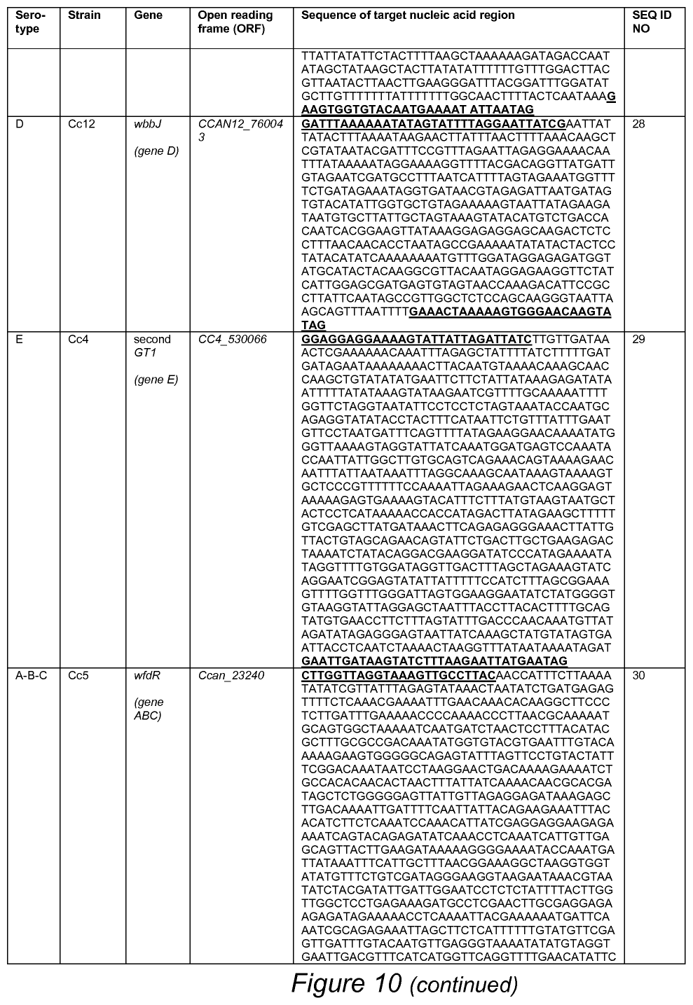

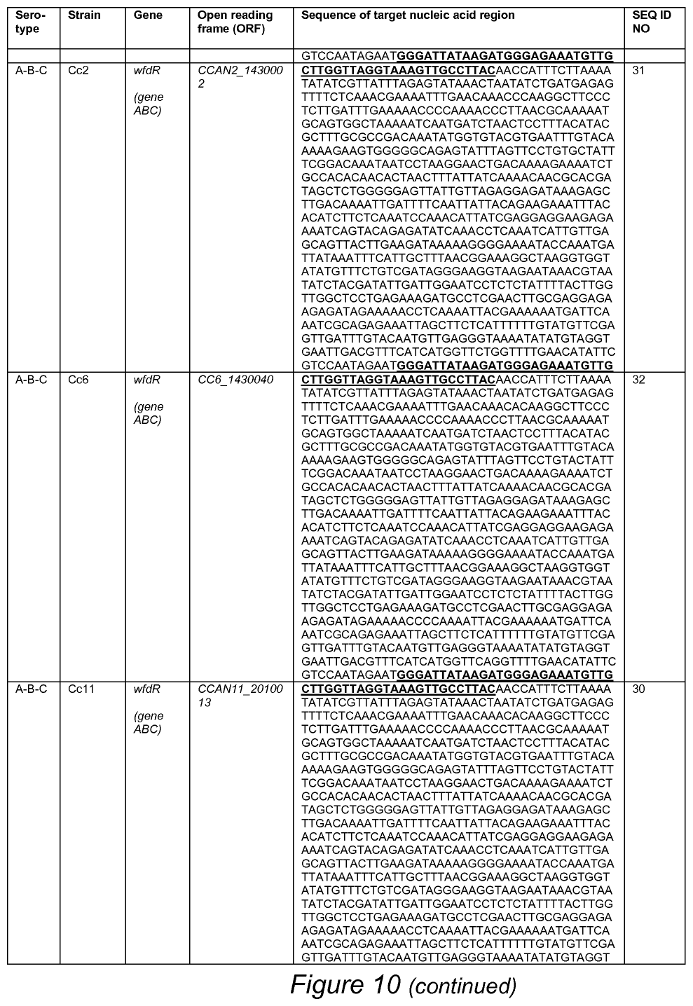

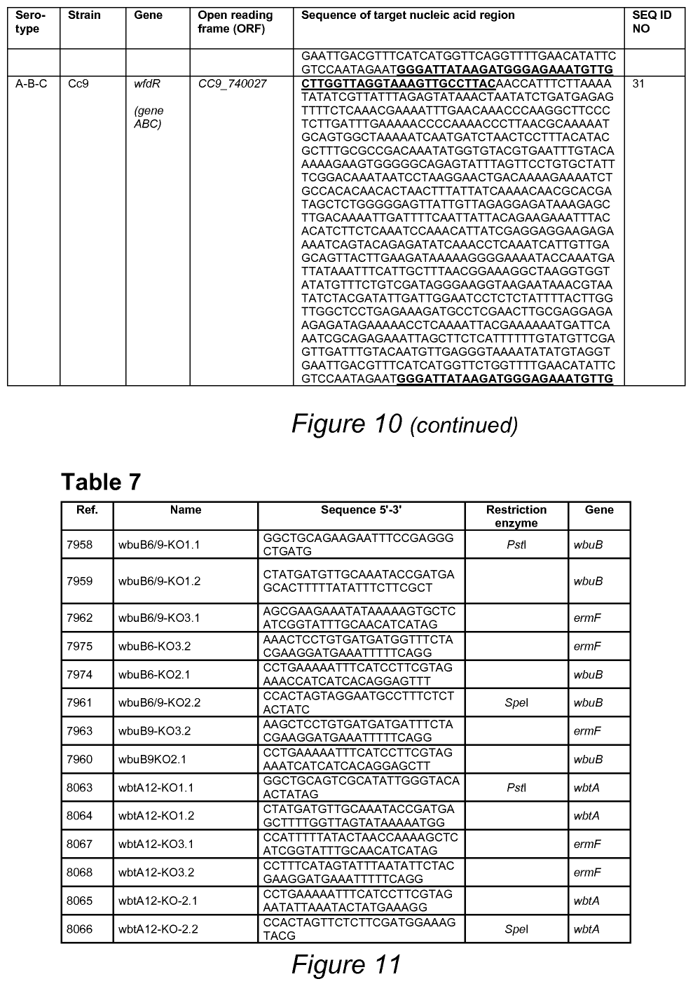

In a particular embodiment, the target nucleic acid region in said gene A, gene B, gene C, gene D, gene E and gene ABC encompasses at least 90%, preferably at least 95%, more preferably at least 96% of the coding nucleic acid sequence of said gene (Table 3, FIG. 10).

In a particular embodiment, the target nucleic acid region in A4galT GT consists of a nucleic acid sequence which is at least 90%, preferably at least 95%, identical to SEQ ID NO: 25; the target nucleic acid region in the first GT1 consists of a nucleic acid sequence which is at least 90%, preferably at least 95%, identical to SEQ ID NO: 26 or 50, the target nucleic acid region in wzy consists of a nucleic acid sequence which is at least 90%, preferably at least 95%, identical to SEQ ID NO: 27, the target nucleic acid region in wbbJ consists of a nucleic acid sequence which is at least 90%, preferably at least 95%, identical to SEQ ID NO: 28, the target nucleic acid region in the second GT1 consists of a nucleic acid sequence which is at least 90%, preferably at least 95%, identical to SEQ ID NO: 29, and/or the target nucleic acid region in wfdR consists of a nucleic acid sequence which is at least 90%, preferably at least 95%, identical to SEQ ID NO: 30, 31 or 32 (Table 3, FIG. 10).

In a particular embodiment, the method for serotyping C. canimorsus or identifying C. canimorsus as one of serotype A, serotype B, serotype C, serotype D or serotype E in a sample as taught herein, comprises the step of
contacting said sample or at least a portion of nucleic acids isolated from said sample under conditions conducive to polymerase-based nucleic acid amplification with an amplification primer pair configured to amplify a target nucleic acid region in C. canimorsus A4galT GT, wherein the coding nucleic acid sequence of said gene is at least 90%, preferably at least 95%, more preferably 100% identical to SEQ ID NO: 1, an amplification primer pair configured to amplify a target nucleic acid region in a first C. canimorsus GT1, wherein the coding nucleic acid sequence of said gene is at least 90%, preferably at least 95%, more preferably 100% identical to SEQ ID NO: 2 and/or 49; an amplification primer pair configured to amplify a target nucleic acid region in C. canimorsus wzy, wherein the coding nucleic acid sequence of said gene is at least 90%, preferably at least 95%, more preferably 100% identical to SEQ ID NO: 3, an amplification primer pair configured to amplify a target nucleic acid region in C. canimorsus wbbJ; wherein the coding nucleic acid sequence of said gene is at least 90%, preferably at least 95%, more preferably 100% identical to SEQ ID NO: 4 and an amplification primer pair configured to amplify a second C. canimorsus GT1, wherein the coding nucleic acid sequence of said gene is at least 90%, preferably at least 95%, more preferably 100% identical to SEQ ID NO: 5; and one or more of the following steps:
detecting the amplified target nucleic acid region in A4galT GT, whereby the presence of C. canimorsus capsular serotype A in the sample is detected or whereby C. canimorsus is identified as serotype A;
detecting the amplified target nucleic acid region in the first GT1 and simultaneously not detecting the amplified target nucleic acid region in A4galT GT, whereby the presence of C. canimorsus capsular serotype B in the sample is detected or whereby C. canimorsus is identified as serotype B;
detecting the amplified target nucleic acid region in wzy and simultaneously not detecting the amplified target nucleic acid region in the A4galT GT, whereby the presence of C. canimorsus capsular serotype C in the sample is detected or whereby C. canimorsus is identified as serotype C;
detecting the amplified target nucleic acid region in wbbJ, whereby the presence of C. canimorsus capsular serotype D in the sample is detected or whereby C. canimorsus is identified as serotype D; or
detecting the amplified target nucleic acid region in the second GT1, whereby the presence of C. canimorsus capsular serotype E in the sample is detected or whereby C. canimorsus is identified as serotype E. Also provided herein is a more simplified serotyping method or method of identifying C. canimorsus as one of serotype A, serotype B, serotype C, serotype D or serotype E, in which detection reagent, such as one probe or one amplification primer pair is sufficient to detect the presence of a pathogenic C. canimorsus (i.e. capsular serotype A, serotype B or serotype C) in a sample. Indeed, the inventors have found that this nucleic acid region is common to the three serotypes.

In a particular embodiment, the method for serotyping C. canimorsus or identifying C. canimorsus as one of serotype A, serotype B, serotype C, serotype D or serotype E in a sample comprises the steps of:
contacting said sample or at least a portion of nucleic acids isolated from said sample under conditions conducive to polymerase-based nucleic acid amplification with an amplification primer pair configured to amplify a target nucleic acid region in C. canimorsus wfdR, wherein the coding nucleic acid sequence of said gene is at least 90%, preferably at least 95%, more preferably 100% identical to SEQ ID NO: 6, SEQ ID NO:7 and/or SEQ ID NO:8; and
detecting the amplified target nucleic acid region in wfdR whereby the presence of serotype A, B or C in the sample is detected or whereby C. canimorsus is identified as one of serotype A, B or C.

It might be of interest only to specify the capsular serotype of a pathogenic C. canimorsus (i.e. capsular serotype A, B or C), once the presence of a pathogenic C. canimorsus is detected in the sample. Such strategy will avoid unnecessary PCR analyses.

In a particular embodiment, the method for serotyping C. canimorsus or identifying C. canimorsus as one of serotype A, serotype B, serotype C, serotype D or serotype E in a sample comprises the steps of:
contacting a first portion said sample or at least a portion of nucleic acids isolated from said sample under conditions conducive to polymerase-based nucleic acid amplification with an amplification primer pair configured to amplify a target nucleic acid region in a target nucleic acid region in a target nucleic acid region in C. canimorsus wfdR, wherein the coding nucleic acid sequence of said gene is at least 90%, preferably at least 95%, more preferably 100% identical to SEQ ID NO: 6, SEQ ID NO:7 and/or SEQ ID NO:8;
detecting the amplified target nucleic acid region in wfdR, whereby the presence of serotype A, B or C in the sample is detected or whereby the C. canimorsus is identified as one of serotype A, B or C;
contacting a second portion said sample or at least a portion of nucleic acids isolated from said sample under conditions conducive to polymerase-based nucleic acid amplification with an amplification primer pair configured to amplify a target nucleic acid region in C. canimorsus A4galT GT, wherein the coding nucleic acid sequence of said gene is at least 90%, preferably at least 95%, more preferably 100% identical to SEQ ID NO: 1, an amplification primer pair configured to amplify a target nucleic acid region in a first C. canimorsus GT1, wherein the coding nucleic acid sequence of said gene is at least 90%, preferably at least 95%, more preferably 100% identical to SEQ ID NO: 2 and/or 49; an amplification primer pair configured to amplify a target nucleic acid region in C. canimorsus wzy, wherein the coding nucleic acid sequence of said gene is at least 90%, preferably at least 95%, more preferably 100% identical to SEQ ID NO: 3, and one or more of the following steps:
detecting the amplified target nucleic acid region in A4galT GT, whereby the presence of C. canimorsus capsular serotype A in the sample is detected or whereby the C. canimorsus is identified as serotype A;
detecting the amplified target nucleic acid region in the first GT1 and simultaneously not detecting the amplified target nucleic acid region in A4galT GT, whereby the presence of C. canimorsus capsular serotype B in the sample is detected or whereby the C. canimorsus is identified as serotype B; and/or
detecting the amplified target nucleic acid region in wzy and simultaneously not detecting the amplified target nucleic acid region in the A4galT GT, whereby the presence of C. canimorsus capsular serotype C in the sample is detected or whereby the C. canimorsus is identified as serotype C.

In a particular embodiment, the detection of the presence of C. canimorsus serotype A, serotype B or serotype C or the identification of C. canimorsus as one of serotype A is indicative of the presence of a high-pathogenicity C. canimorsus strain.

In a particular embodiment, at most one C. canimorsus serotype selected from the list comprising capsular serotypes A, B, C, D and E, is detected or C. canimorsus is identified to be at most one of the capsular serotypes A, B, C, D and E.

In a particular embodiment, the method for serotyping C. canimorsus or identifying C. canimorsus as one of serotype A, serotype B, serotype C, serotype D or serotype E in a sample as described herein is performed before or after, preferably after, detecting the presence of C. canimorsus in a sample by any method known to the skilled person or as described herein, for example by a C. canimorsus-specific 16S ribosomal DNA PCR.

Because it is understood that nucleic acids do not require complete complementarity in order to hybridize, the primer sequences disclosed herein may be modified to some extent without loss of utility as specific primers. Accordingly, the primers comprised within an amplification primer pair configured to amplify a target nucleic acid region as described herein may comprise a nucleic acid sequence which is substantially identical (i.e., largely but not wholly identical) to the nucleic acid sequence of an amplification primer as set forth in Table 4 (SEQ ID Nos: 9 to 24). For instance, at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical to a nucleic acid sequence of an amplification primer as set forth in Table 4 (SEQ ID Nos: 9 to 24). The skilled person will understand that the primers comprised within the amplification primer pair configured to amplify the target nucleic acid regions as described herein may further comprise additional oligonucleotide sequence(s) serving other useful purpose(s) as described elsewhere herein.

TABLE 4

| Detectable | Forward/ | | Target | SEQ ID |
|---|---|---|---|---|
| serotype | reverse | Sequence 5′-3′ | gene | NO. |
| A | forward | AAAAAAGTACCAATAG | A4galT | 9 |
| | | TTTTTATATTTAACC | GT | |
| A | reverse | TCATTTTTTTATCTTT | A4galT | 10 |
| | | TTTAATATATTCCAC | GT | |
| A | forward | CATACCATGGGAAAAA | A4galT | 11 |
| | | AAGTACCAATAGTTTT | GT | |
| | | TATATTTAACC | | |
| A | reverse | CCGCTCGAGTCATTTT | A4galT | 12 |
| | | TTTATCTTTTTTAATA | GT | |
| | | TATTCCAC | | |
| B | forward | ATTAACAAAATTCTAA | first | 13 |
| | | TAG | GT1 | |
| B | reverse | TTATTTTTTATTTTCA | first | 14 |
| | | TTAG | GT1 | |
| B | forward | CATACCATGGGAATTA | first | 15 |
| | | ACAAAATTCTAATAG | GT1 | |
| B | reverse | CCGCTCGAGTTATTTT | first | 16 |
| | | TTATTTTCATTAG | GT1 | |
| C | forward | GGCGTATATCGTTGCT | wzy | 17 |
| | | ATTTTGTATG | | |
| C | reverse | CTATTAATATTTTCAT | wzy | 18 |
| | | TGTACACCACTTC | | |
| D | forward | GATTTAAAAAATATAG | wbbJ | 19 |
| | | TATTTTAGGAATTATCG | | |
| D | reverse | CTATACTTGTTCCCAC | wbbJ | 20 |
| | | TTTTTAGTTTC | | |
| E | forward | GGAGGAGGAAAAGTAT | second | 21 |
| | | TATTAGATTATC | GT1 | |
| E | reverse | CTATTCATAATTCTTA | second | 22 |
| | | AAGATACTTATCAATTC | GT1 | |
| A, B, C | forward | CTTGGTTAGGTAAAGT | wfdR | 23 |
| | | TGCCTTAC | | |
| A, B, C | reverse | CAACATTTCTCCCATC | wfdR | 24 |
| | | TTATAATCCC | | |

Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (Altschul et al., J Mol Biol, 1990, 215: 403-10). An example procedure to determine the percent identity between a particular nucleic acid sequence and the nucleic acid sequence of a query oligonucleotide sequence (e.g., an amplification primer as set forth in SEQ ID NO: 9) will entail aligning the two nucleic acid sequences using Nucleotide-nucleotide BLAST (blastn) or Primer-BLAST, available as a web application at the NCBI web site (www.ncbi.nlm.nih.gov), using suitable algorithm parameters. If the two compared sequences do not share homology, then the output will not present aligned sequences. Once aligned, the number of matches will be determined by counting the number of positions where an identical nucleic acid residue is presented in both sequences. The percent identity is determined by dividing the number of matches by the length of the query polynucleotide, followed by multiplying the resulting value by 100. The percent identity value may, but need not, be rounded to the nearest tenth. For example, 78.11, 78.12, 78.13, and 78.14 may be rounded down to 78.1, while 78.15, 78.16, 78.17, 78.18, and 78.19 may be rounded up to 78.2.

In a particular embodiment, the following characteristics may apply in respect of the amplification primer pairs as taught herein:
the amplification primer pair configured to amplify a target nucleic acid region in A4galT GT comprises a first amplification primer which comprises a nucleotide sequence which is at least 90%, preferably at least 95%, more preferably 100% identical to SEQ ID NO: 9, i.e. 5′-AAAAAAGTACCAATAGTTTTTATATTTAACC-3′ (SEQ ID NO: 9) or SEQ ID NO: 11, i.e. 5′-CATACCATGGGAAAAAAAGTACCAATAGTTTTTATATTTAACC-3′ (SEQ ID NO: 11) and a second amplification primer which comprises a nucleotide sequence which is at least 90%, preferably at least 95%, more preferably 100% identical to SEQ ID NO: 10, i.e. 5′-TCATTTTTTTATCTTTTTTAATATATTCCAC-3′ (SEQ ID NO: 10) or to SEQ ID NO: 12, i.e. 5′-CCGCTCGAGTCATTTTTTTATCTTTTTTAATATATTCCAC-3′ (SEQ ID NO: 12);
the amplification primer pair configured to amplify a target nucleic acid region in the first GT1 comprises a first amplification primer which comprises a nucleotide sequence which is at least 90%, preferably at least 95%, more preferably 100% identical to SEQ ID NO: 13, i.e. 5′-ATTAACAAAATTCTAATAG-3′ (SEQ ID NO: 13) or SEQ ID NO: 15, i.e. 5′-CATACCATGGGAATTAACAAAATTCTAATAG-3′ (SEQ ID NO: 15) and a second amplification primer which comprises a nucleotide sequence which is at least 90%, preferably at least 95%, more preferably 100% identical to SEQ ID NO: 14, i.e. 5′-TTATTTTTTATTTTCATTAG-3′ (SEQ ID NO: 14) or SEQ ID NO: 16, i.e. 5′-CCGCTCGAGTTATTTTTTATTTTCATTAG-3′ (SEQ ID NO: 16);
the amplification primer pair configured to amplify a target nucleic acid region in wzy comprises a first amplification primer which comprises a nucleotide sequence which is at least 90%, preferably at least 95%, more preferably 100% identical to SEQ ID NO: 17, i.e. 5′-GGCGTATATCGTTGCTATTTTGTATG-3′ (SEQ ID NO: 17) and a second amplification primer which comprises a nucleotide sequence which is at least 90%, preferably at least 95%, identical to SEQ ID NO: 18, i.e. 5′-CTATTAATATTTTCATTGTACACCACTTC-3′ (SEQ ID NO: 18);
the amplification primer pair configured to amplify a target nucleic acid region in wbbJ comprises a first amplification primer which comprises a nucleotide sequence which is at least 90%, preferably at least 95%, more preferably 100% identical to SEQ ID NO: 19, i.e. 5′-GATTTAAAAAATATAGTATTTTAGGAATTATCG-3′ (SEQ ID NO: 19) and a second amplification primer which comprises a nucleotide sequence which is at least 90%, preferably at least 95%, more preferably 100% identical to SEQ ID NO: 20, i.e. 5′-CTATACTTGTTCCCACTTTTTAGTTTC-3′ (SEQ ID NO: 20);
the amplification primer pair configured to amplify a target nucleic acid region in the second GT1 comprises a first amplification primer which comprises a nucleotide sequence which is at least 90%, preferably at least 95%, more preferably 100% identical to SEQ ID NO: 21, i.e. 5′-GGAGGAGGAAAAGTATTATTAGATTATC-3′ (SEQ ID NO: 21) and a second amplification primer which comprises a nucleotide sequence which is at least 90%, preferably at least 95%, more preferably 100% identical to SEQ ID NO: 22, i.e. 5′-CTATTCATAATTCTTAAAGATACTTATCAATTC-3′ (SEQ ID NO: 22); and/or
the amplification primer pair configured to amplify a target nucleic acid region in wfdR comprises a first amplification primer which comprises a nucleotide sequence which is at least 90%, preferably at least 95%, more preferably 100% identical to SEQ ID NO: 23, i.e. 5′-CTTGGTTAGGTAAAGTTGCCTTAC-3′ (SEQ ID NO: 23) and a second amplification primer which comprises a nucleotide sequence which is at least 90%, preferably at least 95%, more preferably 100% identical to SEQ ID NO: 24, i.e. 5′-CAACATTTCTCCCATCTTATAATCCC-3′ (SEQ ID NO: 24).

In a particular embodiment, the following characteristics may apply in respect of the amplification primer pairs as taught herein:
the amplification primer pair configured to amplify a target nucleic acid region in A4galT GT comprises the amplification primer set forth in SEQ ID NO: 11, i.e. 5′-CATACCATGGGAAAAAAAGTACCAATAGTTTTTATATTTAACC-3′ (SEQ ID NO: 11) and the amplification primer set forth in SEQ ID NO: 12, i.e. 5′-CCGCTCGAGTCATTTTTTTATCTTTTTTAATATATTCCAC-3′ (SEQ ID NO: 12);
the amplification primer pair configured to amplify a target nucleic acid region in the first GT1 comprises the amplification primer set forth in SEQ ID NO: 15, i.e. 5′-CATACCATGGGAATTAACAAAATTCTAATAG-3′ (SEQ ID NO: 15) and the amplification primer set forth in SEQ ID NO: 16, i.e. 5′-CCGCTCGAGTTATTTTTTATTTTCATTAG-3′ (SEQ ID NO: 16);
the amplification primer pair configured to amplify a target nucleic acid region in wzy comprises the amplification primer set forth in SEQ ID NO: 17, i.e. 5′-GGCGTATATCGTTGCTATTTTGTATG-3′ (SEQ ID NO: 17) and the amplification primer set forth in SEQ ID NO: 18, i.e. 5′-CTATTAATATTTTCATTGTACACCACTTC-3′ (SEQ ID NO: 18);
the amplification primer pair configured to amplify a target nucleic acid region in wbbJ comprises the amplification primer set forth in SEQ ID NO: 19, i.e. 5′-GATTTAAAAAATATAGTATTTTAGGAATTATCG-3′ (SEQ ID NO: 19) and the amplification primer set forth in SEQ ID NO: 20, i.e. 5′-CTATACTTGTTCCCACTTTTTAGTTTC-3′ (SEQ ID NO: 20);
the amplification primer pair configured to amplify a target nucleic acid region in the second GT1 comprises the amplification primer set forth in SEQ ID NO: 21, i.e. 5′-GGAGGAGGAAAAGTATTATTAGATTATC-3′ (SEQ ID NO: 21) and the amplification primer set forth in SEQ ID NO: 22, i.e. 5′-CTATTCATAATTCTTAAAGATACTTATCAATTC-3′ (SEQ ID NO: 22); and/or
the amplification primer pair configured to amplify a target nucleic acid region in wfdR comprises the amplification primer set forth in SEQ ID NO: 23, i.e. 5′-CTTGGTTAGGTAAAGTTGCCTTAC-3′(SEQ ID NO: 23) and the amplification primer set forth in SEQ ID NO: 24, i.e. 5′-CAACATTTCTCCCATCTTATAATCCC-3′ (SEQ ID NO: 24).

A further aspect provides in a set of amplification primer pairs suitable for polymerase-based nucleic acid amplification, comprising an amplification primer pair configured to amplify a target nucleic acid region in A4galT GT, an amplification primer pair configured to amplify a target nucleic acid region in a first GT1, an amplification primer pair configured to amplify a target nucleic acid region in wzy, an amplification primer pair configured to amplify a target nucleic acid region in wbbJ, an amplification primer pair configured to amplify a target nucleic acid region in a second GT1 and/or an amplification primer pair configured to amplify a target nucleic acid region in wfdR.

In a particular embodiment, any one of the following characteristics may apply in respect of the set of amplification primer pairs as taught herein:
the amplification primer pair configured to amplify a target nucleic acid region in A4galT GT comprises a first amplification primer which comprises a nucleotide sequence which is at least 90%, preferably at least 95%, identical to SEQ ID NO: 9, i.e. 5′-AAAAAAGTACCAATAGTTTTTATATTTAACC-3′ (SEQ ID NO: 9) or to SEQ ID NO: 11, i.e. 5′-CATACCATGGGAAAAAAAGTACCAATAGTTTTTATATTTAACC-3′ (SEQ ID NO: 11) and a second amplification primer which comprises a nucleotide sequence which is at least 90%, preferably at least 95%, identical to SEQ ID NO: 10, i.e. 5′-TCATTTTTTTATCTTTTTTAATATATTCCAC-3′ (SEQ ID NO: 10) or to SEQ ID NO: 12, i.e. 5′-CCGCTCGAGTCATTTTTTTATCTTTTTTAATATATTCCAC-3′ (SEQ ID NO: 12);
the amplification primer pair configured to amplify a target nucleic acid region in the first GT1 comprises a first amplification primer which comprises a nucleotide sequence which is at least 90%, preferably at least 95%, identical to SEQ ID NO: 13, i.e. 5′-ATTAACAAAATTCTAATAG-3′ (SEQ ID NO: 13) or to SEQ ID NO: 15, i.e. 5′-CATACCATGGGAATTAACAAAATTCTAATAG-3′ (SEQ ID NO: 15) and a second amplification primer which comprises a nucleotide sequence which is at least 90%, preferably at least 95%, identical to SEQ ID NO: 14, i.e. 5′-TTATTTTTTATTTTCATTAG-3′ (SEQ ID NO: 14) or to SEQ ID NO: 16, i.e. 5′-CCGCTCGAGTTATTTTTTATTTTCATTAG-3′ (SEQ ID NO: 16);
the amplification primer pair configured to amplify a target nucleic acid region in wzy comprises a first amplification primer which comprises a nucleotide sequence which is at least 90%, preferably at least 95%, identical to SEQ ID NO: 17, i.e. 5′-GGCGTATATCGTTGCTATTTTGTATG-3′ (SEQ ID NO: 17) and a second amplification primer which comprises a nucleotide sequence which is at least 90%, preferably at least 95%, identical to SEQ ID NO: 18, i.e. 5′-CTATTAATATTTTCATTGTACACCACTTC-3′ (SEQ ID NO: 18);
the amplification primer pair configured to amplify a target nucleic acid region in wbbJ comprises a first amplification primer which comprises a nucleotide sequence which is at least 90%, preferably at least 95%, identical to SEQ ID NO: 19, i.e. 5′-GATTTAAAAAATATAGTATTTTAGGAATTATCG-3′ (SEQ ID NO: 19) and a second amplification primer which comprises a nucleotide sequence which is at least 90%, preferably at least 95%, identical to SEQ ID NO: 20, i.e. 5′-CTATACTTGTTCCCACTTTTTAGTTTC-3′ (SEQ ID NO: 20);
the amplification primer pair configured to amplify a target nucleic acid region in the second GT1 comprises a first amplification primer which comprises a nucleotide sequence which is at least 90%, preferably at least 95%, identical to SEQ ID NO: 21, i.e. 5′-GGAGGAGGAAAAGTATTATTAGATTATC-3′ (SEQ ID NO: 21) and a second amplification primer which comprises a nucleotide sequence which is at least 90%, preferably at least 95%, identical to SEQ ID NO: 22, i.e. 5′-CTATTCATAATTCTTAAAGATACTTATCAATTC-3′ (SEQ ID NO: 22); and/or
the amplification primer pair configured to amplify a target nucleic acid region in wfdR comprises a first amplification primer which comprises a nucleotide sequence which is at least 90%, preferably at least 95%, identical to SEQ ID NO: 23, i.e. 5′-CTTGGTTAGGTAAAGTTGCCTTAC-3′ (SEQ ID NO: 23) and a second amplification primer which comprises a nucleotide sequence which is at least 90%, preferably at least 95%, identical to SEQ ID NO: 24, i.e. 5′-CAACATTTCTCCCATCTTATAATCCC-3′ (SEQ ID NO: 24).

In a particular embodiment, any one of the following characteristics may apply in respect of the set of amplification primer pairs as taught herein:
the amplification primer pair configured to amplify a target nucleic acid region in A4galT GT comprises the amplification primer set forth in SEQ ID NO: 11, i.e. 5′-CATACCATGGGAAAAAAAGTACCAATAGTTTTTATATTTAACC-3′ (SEQ ID NO: 11) and the amplification primer set forth in SEQ ID NO: 12, i.e. 5′-CCGCTCGAGTCATTTTTTTATCTTTTTTAATATATTCCAC-3′ (SEQ ID NO: 12);
the amplification primer pair configured to amplify a target nucleic acid region in the first GT1 comprises the amplification primer set forth in SEQ ID NO: 15, i.e. 5′-CATACCATGGGAATTAACAAAATTCTAATAG-3′ (SEQ ID NO: 15) and the amplification primer set forth in SEQ ID NO: 16, i.e. 5′-CCGCTCGAGTTATTTTTTATTTTCATTAG-3′ (SEQ ID NO: 16);
the amplification primer pair configured to amplify a target nucleic acid region in wzy comprises the amplification primer set forth in SEQ ID NO: 17, i.e. 5′-GGCGTATATCGTTGCTATTTTGTATG-3′ (SEQ ID NO: 17) and the amplification primer set forth in SEQ ID NO: 18, i.e. 5′-CTATTAATATTTTCATTGTACACCACTTC-3′ (SEQ ID NO: 18);
the amplification primer pair configured to amplify a target nucleic acid region in wbbJ comprises the amplification primer set forth in SEQ ID NO: 19, i.e. 5′-GATTTAAAAAATATAGTATTTTAGGAATTATCG-3′ (SEQ ID NO: 19) and the amplification primer set forth in SEQ ID NO: 20, i.e. 5′-CTATACTTGTTCCCACTTTTTAGTTTC-3′ (SEQ ID NO: 20);
the amplification primer pair configured to amplify a target nucleic acid region in the second GT1 comprises the amplification primer set forth in SEQ ID NO: 21, i.e. 5′-GGAGGAGGAAAAGTATTATTAGATTATC-3′ (SEQ ID NO: 21) and the amplification primer set forth in SEQ ID NO: 22, i.e. 5′-CTATTCATAATTCTTAAAGATACTTATCAATTC-3′ (SEQ ID NO: 22); and/or
the amplification primer pair configured to amplify a target nucleic acid region in wfdR comprises the amplification primer set forth in SEQ ID NO: 23, i.e. 5′-CTTGGTTAGGTAAAGTTGCCTTAC-3′(SEQ ID NO: 23) and the amplification primer set forth in SEQ ID NO: 24, i.e. 5′-CAACATTTCTCCCATCTTATAATCCC-3′ (SEQ ID NO: 24).

The term “set” as in “a set of amplification primer pairs” is synonymous with such terms as “collection”, “group”, “grouping”, “combination”, or “assembly”. The phrase “a set of amplification primer pairs” denotes the set of amplification primer pairs irrespective of whether the primers or primer pairs constituting the set are provided each individually (e.g., each primer or primer pair may be provided within a separate composition), or are provided as several sub-sets together making up the set (e.g., each sub-set of primers or primer pairs may be provided within a separate composition), or are provided as a complete set (e.g., the set of primer pairs may be provided within the same composition).

A further aspect provides in a composition comprising the set of amplification primer pairs as taught herein. In a particular embodiments, compositions comprising amplification primers or primer pairs may contain additional components, in particular components compatible with the use of the primers or primer pairs in polymerase-based amplification methods, such as, without limitation, solvents, buffers, salts, or similar.

In a particular embodiment, the amplification primer pairs as described herein may be configured to allow for multiplexed polymerase-based nucleic acid amplification, such that at least two, at least three, at least four, or at least five and most preferably all six of the target nucleic acid regions can be amplified in the same polymerase-based nucleic acid amplification reaction.

A further aspect provides in a set of oligonucleotide probes comprising oligonucleotide probes configured to specifically hybridise with a target nucleic acid region in A4galT GT, oligonucleotide probes configured to specifically hybridise with a target nucleic acid region in a first GT1, oligonucleotide probes configured to specifically hybridise with a target nucleic acid region in wzy, oligonucleotide probes configured to specifically hybridise with a target nucleic acid region in wbbJ, oligonucleotide probes configured to specifically hybridise with a target nucleic acid region in a second GT1 and/or oligonucleotide probes configured to specifically hybridize with a target nucleic acid region in wfdR.

In a particular embodiment, the primers and/or oligonucleotide probes as taught herein may comprise detectable labels. Preferably, the detectable labels may allow for individual detection and monitoring of each of the amplified target nucleic acid regions. Preferably the detectable labels may comprise distinct fluorophores having distinct excitation and/or emission characteristics, such that each of the amplified target nucleic acid regions can be individually detected by detecting the corresponding fluorophore.

The term “label” or “labelled” refers to any atom, molecule, moiety or biomolecule that can be used to provide a detectable and preferably quantifiable read-out or property, and that can be attached to or made part of an entity of interest, such as a primer. Labels may be suitably detectable by mass spectrometric, spectroscopic, optical, colorimetric, magnetic, photochemical, biochemical, immunochemical or chemical means. A wide variety of labels and conjugation techniques, including direct and indirect labelling, are known and are reported extensively in both the scientific and patent literature. Examples of labels that can be used include radionucleotides, enzymes, substrates, cofactors, inhibitors, fluorescent moieties, intercalators, chemiluminescent moieties, magnetic particles, and the like. For example, labels include without limitation a radioactive isotope (e.g., ³²P, ³³P), ligand, chemiluminescent agent, fluorophore (e.g., fluorescein, tetrachloro-fluorescein, TAMRA, ROX, Cy3, Cy3.5, Cy5, Cy5.5, Texas Red, etc.), vitamin (e.g., biotin), steroid (e.g., digoxin), enzyme (e.g., HRP, AP, etc.), etc.

Another aspect provides in a kit of parts comprising the set of amplification primer pairs and/or the set of oligonucleotide probes as described above, and optionally further comprising reagents sufficient for formulating a polymerase-based nucleic acid amplification reaction mixture. For example, such reagents may include one or more or all of thermostable nucleic acid polymerase, preferably thermostable DNA polymerase, such as without limitation Taq polymerase, a mixture of nucleotides, preferably deoxynucleotides (dATP, dGTP, dCTP, dTTP), a suitable reaction buffer, source of divalent ions, preferably Mg²⁺ ions, such as magnesium sulphate, and deionised water. The kits may further comprise instructions for using the provided composition in a polymerase-based amplification reaction. Kits as intended herein may comprise a carrier being compartmentalised to receive in close confinement therein one or more containers, such as tubes or vials. The containers will hold the set of amplification primer pairs as taught herein. The primers may be present in lyophilised form or in an appropriate buffer as necessary. One or more containers may contain one or more enzymes or reagents to be utilised in amplification reactions. These enzymes may be present by themselves or in admixtures, in lyophilized form or in appropriate buffers. The kit may optionally contain any or all additional elements useful to carry out the techniques taught herein, such as buffers, extraction reagents, enzymes, pipettes, plates, nucleic acids, nucleoside triphosphates, filter paper, gel materials, transfer materials, autoradiography supplies, and the like. The various reagent components of the kits may be present in separate containers, or may some or all be pre-combined into a reagent mixture for combination with template nucleic acid.

Instructions for using the provided composition in a polymerase-based amplification reaction may be included in the kits; such as in any one or more of a variety of forms. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, flash memory, etc., on which the information has been recorded. Yet another means that may be present is a website address that may be used via the internet to access the information at a removed site. Any convenient means may be present in the kits.

Also provided herein are antibodies capable of specifically detecting one capsular serotype of C. canimorsus, i.e. capsular serotype A, capsular serotype B, capsular serotype C, capsular serotype D or capsular serotype E. Indeed, given that these capsular serotypes have been identified, it is now possible to generate antibodies and screen them for specificity against a specific serotype. Methods for producing antibodies are known in the art. In particular embodiments, the antibodies are polyclonal antibodies. In further particular embodiments, the polyclonal antibodies are obtained by adsorbing anti-serum obtained by immunization with (fixed) C. canimorsus bacteria with (fixed) non-capsulated and rough mutant C. canimorsus bacteria or by absorbing anti-serum obtained by immunization with (fixed) C. canimorsus bacteria subsequently or simultaneously with at least one, preferably all, wild-type C. canimorsus strain(s) of the capsular serotype A, at least one, preferably all, wild-type C. canimorsus strain(s) of the capsular serotype B, at least one, preferably all, wild-type C. canimorsus strain(s) of the capsular serotype C and at least one, preferably all, wild-type C. canimorsus strain(s) of the capsular serotype D and at least one, preferably all, wild-type C. canimorsus strain(s) of the capsular serotype E; with the proviso that if the anti-serum is adsorbed with wild-type C. canimorsus strains, the anti-serum is not adsorbed with C. canimorsus strains of the same capsular serotype as used for immunization. As detailed herein, the antibodies may be capable of detecting other Capnocytophaga species of the same serotype.

In particular embodiments, the wild-type C. canimorsus strain(s) of the capsular serotype A are Cc1, Cc2, Cc3, Cc5, Cc10, Cc13, Cc15, Cc21, Cc22, Cc24 and Cc25, the wild-type C. canimorsus strain(s) of the capsular serotype B are Cc6, Cc8, Cc11, Cc16, Cc17, Cc18 and Cc23, the wild-type C. canimorsus strain(s) of the capsular serotype C are Cc9, Cc14, Cc19 and Cc20, the wild-type C. canimorsus strain(s) of the capsular serotype D are Cc12 and Cc7, the wild-type C. canimorsus strain(s) of the capsular serotype A is Cc4.

The term “non-capsulated and rough mutant”, as used herein, refers to C. canimorsus bacteria missing a capsule and at least part of or the complete LOS. Such mutants can be obtained by all methods known in the art, for example by inactivation and/or deletion of genes involved in the biosynthesis of said capsule and LOS. Non-limiting examples of non-capsulated and rough mutant C. canimorsus bacteria are a C. canimorsus 5 Y1C12 mutant which is non-capsulated and presents an incomplete LOS (Renzi F. et al, Scientific Reports, 2016) (i.e. inactivation of wbuB, which is a gene encoding a N-acetyl-fucosamine (FucNAc) transferase), a C. canimorsus 6 ΔwbuB mutant (i.e. inactivation of wbuB), a C. canimorsus 9 ΔwbuB mutant (i.e. inactivation of wbuB) or a C. canimorsus 12 ΔwbtA mutant (i.e. inactivation of wbtA, which is a gene putatively encoding a UDP-N-acetylglucosamine 4,6-dehydratase).

The term “polyclonal antibody”, as used herein, may refer to an anti-serum or immunoglobulins purified therefrom (e.g., affinity-purified). The term “anti-serum” refers to blood serum isolated from one or more non-human animals containing polyclonal antibodies. In the context of the present invention, polyclonal antibodies are provided which are directed against one or more C. canimorsus strains and preferably specifically react with one or more specific C. canimorsus strains. In this context, one or more C. canimorsus strains are used to immunize a non-human animal(s). Anti-serum may be produced by any method known in the art. For example, anti-serum may be produced by growing bacteria of the selected one or more C. canimorsus strains on sheep blood plates, inoculating a non-human animal (e.g. a rabbit) with the bacteria after resuspending, fixating and washing them, and centrifuging blood collected from the inoculated non-human animal to retrieve the polyclonal anti-serum. Immunization of the non-human animal may be performed once or several times. The amount of C. canimorsus bacteria used for immunization is preferably at least 1.5×10⁸/kg body weight, at least 2×10⁸/kg body weight or at least 2.5×10⁸/kg body weight. The anti-serum is preferably subjected to an adsorption method to deplete the antibodies recognizing structures other than the CPS/LOS and/or recognizing structures other than the CPS/LOS of the one or more bacterial strains, more particularly deplete the antibodies recognizing structures other than the CPS/LOS of the strain of interest. The IgG fraction may be collected from the adsorbed anti-serum through methods known in the art such as affinity purification (by use of Protein A or the like) or ion-exchange resin. If required, an additional purification procedure (e.g. purification through gel filteration) may be performed in combination.

Adsorption may be performed by incubating the polyclonal antiserum with an excess of fixed non-capsulated, and rough (i.e. no or at least an incomplete LOS), mutant C. canimorsus bacteria. Incubation may be performed on a rotating wheel for at least 2, at least 3 or at least 4 times to ensure that only the anti-CPS/LOS antibodies remain in the treated anti-serum and is preferably performed at room temperature. During this incubation antibodies recognizing any other epitopes than the ones present in the CPS/LOS, bind to the mutant bacteria. Bacteria may be removed after the adsorption method by centrifugation. Adsorption efficacy is preferably 70%, 75%, 80%, 85%, 90%, 95%, 96%, 97%, 98%, 99%, 100% and may be assessed by methods known in the art, such as immunofluorescence staining, western blot analysis and/or enzyme-linked immunosorbent assay (ELISA). For example, for immunofluorescence staining, glass coverslips may be coated with poly-D-lysine, washed and incubated for 30 minutes at 37° C. with a bacterial suspension adjusted to an OD₆₀₀ of 0.25. Next, the coverslips are washed and bacteria are fixed for 15 min with 4% PFA. Coverslips are washed again and blocked with 1% bovine serum albumin (BSA) for 1 hour at RT. Subsequently, bacteria may be stained with the adsorbed sera for 1 hour at RT followed by an incubation with a fluorescently-labeled anti-rabbit antibody for 45 min. In particular embodiments, the application provides polyclonal antibodies capable of specifically detecting the presence of bacteria of one or more non-mutated C. canimorsus strains, which do not detect non-capsid components. The application demonstrates methods which encompass immunization of a non-human animal with a composition comprising one or more C. canimorsus strains and that adsorption of the antiserum so-obtained with non-capsulated and rough mutant of a C. canimorsus strain, preferably with a non-capsulated and rough mutant of the C. canimorsus strain(s) used for immunization prevents cross-reaction with non-capsular proteins or sugars of C. canimorsus. In these embodiments, adsorption is effective if the adsorbed anti-serum is able to detect the bacteria of the one or more non-mutated C. canimorsus strain which was used for immunizing, but not the non-capsulated and rough mutant thereof. As detailed herein the antibodies may also detect the presence of other Capnocytophaga bacteria of the same serotype. Accordingly, the application in fact provides polyclonal antibodies capable of specifically detecting the presence of bacteria of one or more non-mutated Capnocytophaga bacteria. However, while these antibodies may also recognize other Capnocytophaga bacteria given the prevalence of and the relevance of C. canimorsus in many applications, these antibodies can nevertheless still be used to detect C. canimorsus serotypes. Where a more specific identification is required, a combination with a further identification method, such as described herein is envisaged.

In order to be specific for the capsular proteins, the antibodies are preferably adsorbed with non-capsulated and rough mutant bacteria after immunization.

In a particular embodiment, the non-capsulated and rough mutant C. canimorsus bacteria have the same serotype as the C. canimorsus strain used for immunization.

In a particular embodiment, the non-capsulated and rough mutant C. canimorsus bacteria are mutant forms of the C. canimorsus strain used for immunization. Accordingly, in a particular embodiment, the non-capsulated and rough mutant C. canimorsus bacterium is a C. canimorsus 5 Y1C12 mutant if the C. canimorsus used for immunization is C. canimorsus 5, the non-capsulated and rough mutant C. canimorsus bacterium is a C. canimorsus 6 ΔwbuB mutant if the C. canimorsus used for immunization is C. canimorsus 6, non-capsulated and rough mutant C. canimorsus bacterium is a C. canimorsus 9 ΔwbuB mutant if the C. canimorsus used for immunization is C. canimorsus 9 and non-capsulated and rough mutant C. canimorsus bacterium is a C. canimorsus 12 ΔwbtA mutant if the C. canimorsus used for immunization is C. canimorsus 12.

In particular embodiments, the application provides polyclonal antibodies specifically recognizing the CPS/LOS of C. canimorsus bacteria of one or more (but not all) capsular serotypes of interest and methods for making such antibodies. The application provides methods for ensuring that a polyclonal antibody specifically recognizes the CPS/LOS of C. canimorsus bacteria of only the one or more capsular serotype(s) of the C. canimorsus bacteria used for immunization. The one or more capsular serotypes may be capsular serotype A, B, C, D or E as taught herein.

The inventors have found that polyclonal antibodies obtained by the methods as described herein are able to recognize capsular serotype A, capsular serotype B, capsular serotype C, capsular serotype D or capsular serotype E of not only C. canimorsus, but also other Capnocytophaga bacteria.

Accordingly, in particular embodiments the polyclonal antibodies specifically recognizing the CPS/LOS of Capnocytophaga bacteria of one or more (but not all) capsular serotypes of interest and methods for making such antibodies. As detailed herein, in view of the relevance of the serotyping of C. canimorsus in the context of the present invention, these antibodies can still be considered to specifically recognize the CPS/LOS of C. canimorsus bacteria, in that the serotypes are specifically recognized. Moreover, in view of the relevance of C. canimorsus in certain diagnostic and/or therapeutic application, the antibodies may nevertheless be used as specific for C. canimorsus serotypes (given that the identification of other species may be less relevant in this context).

The methods for obtaining capsular-serotype specific antibodies encompass immunization of a non-human animal with a composition comprising one or more C. canimorsus strains of one or more capsular serotype and adsorption with one or more (fixed) C. canimorsus bacteria of one or more serotypes other than the strain(s) used for immunization. Accordingly, if the immunization is performed with a C. canimorsus strain of capsular serotype A, it can be ensured that it specifically recognizes CPS/LOS of C. canimorsus capsular serotype A, and not of capsular serotype B, C, D and E; if the immunization is performed with a C. canimorsus strain of capsular serotype B, it can be ensured that it specifically recognizes CPS/LOS of Capnocytophaga capsular serotype B, preferably C. canimorsus capsular serotype B, and not of capsular serotype A, C, D and E; if the immunization is performed with a C. canimorsus strain of capsular serotype C, it can be ensured that it specifically recognizes CPS/LOS of Capnocytophaga capsular serotype C, preferably C. canimorsus capsular serotype C, and not of capsular serotype A, B, D and E; if the immunization is performed with a C. canimorsus strain of capsular serotype D, it can be ensured that it specifically recognizes CPS/LOS of Capnocytophaga capsular serotype D, preferably C. canimorsus capsular serotype D, and not of capsular serotype A, B, C and E and if the immunization is performed with a C. canimorsus strain of capsular serotype E, it can be ensured that it specifically recognizes CPS/LOS of Capnocytophaga capsular serotype E, preferably C. canimorsus capsular serotype E, and not of capsular serotype A, B, C and D.

In a particular embodiment, the polyclonal antibody is obtained by adsorbing anti-serum obtained by immunization with (fixed) C. canimorsus 5 bacteria with (fixed) C. canimorsus 5 Y1C12 mutant bacteria and thus specifically recognizes C. canimorsus CPS/LOS of capsular serotype A (e.g. CPS/LOS of Cc1, Cc2, Cc3, Cc5, Cc10, Cc13, Cc15, Cc21, Cc22, Cc24 and Cc25), and preferably not of capsular serotype B, C, D and E.

In a particular embodiment, a polyclonal antibody is obtained by adsorbing anti-serum that has been obtained by immunization with (fixed) C. canimorsus 6 bacteria with an excess of (fixed) C. canimorsus 6 ΔwbuB mutant bacteria and thus specifically recognizes CPS/LOS of Capnocytophaga capsular serotype B, preferably C. canimorsus capsular serotype B (e.g. CPS/LOS of Cc6, Cc8, Cc11, Cc16, Cc17, Cc18 and Cc23), and preferably not of capsular serotype A, C, D and E.

In a particular embodiment, a polyclonal antibody that specifically recognizes CPS/LOS of Capnocytophaga capsular serotype C, preferably C. canimorsus capsular serotype C (e.g. CPS/LOS of Cc9, Cc14, Cc19 and Cc20), and preferably not of capsular serotype A, B, D and E is obtained by adsorbing anti-serum obtained by immunization with (fixed) C. canimorsus 9 bacteria with (fixed) C. canimorsus 9 ΔwbuB mutant bacteria.

In a particular embodiment, a polyclonal antibody that specifically recognizes CPS/LOS of Capnocytophaga capsular serotype D, preferably C. canimorsus capsular serotype D (e.g. CPS/LOS of Cc7 and Cc12), and preferably not of capsular serotype A, B, C and E is obtained by adsorbing anti-serum obtained by immunization with (fixed) C. canimorsus 12 bacteria with (fixed) C. canimorsus 12 ΔwbtA mutant bacteria.

In a particular embodiment, a polyclonal antibody that specifically recognizes CPS/LOS of Capnocytophaga capsular serotype E, preferably C. canimorsus capsular serotype E (e.g. CPS/LOS of Cc4) and preferably not of capsular serotype A, B, C and D is obtained by adsorbing anti-serum obtained by immunization with (fixed) C. canimorsus 4 bacteria with a mixture of (fixed) C. canimorsus bacteria comprising bacteria of C. canimorsus strain Cc1, Cc2, Cc3, Cc5, Cc6, Cc7, Cc8, Cc9, Cc10, Cc11, Cc12, Cc13, Cc14, Cc15, Cc16, Cc17, Cc18, Cc19, Cc20, Cc21, Cc22, Cc23, Cc24 and Cc25.

The application further provides polyclonal antibodies which specifically recognize CPS/LOS of one or more Capnocytophaga capsular serotypes, preferably C. canimorsus capsular serotypes, but not of all Capnocytophaga capsular serotypes, preferably not all C. canimorsus capsular serotypes and which, in addition, are specific in that they do not recognize other, non-capsular components of Capnocytophaga bacteria, preferably C. canimorsus bacteria, and methods for producing them. Indeed, the adsorption, simultaneously or sequentially with a non-capsulated and rough mutant strain (such as a non-capsulated and rough mutant strain of the strain of interest). Accordingly, methods are provided for the production of such antibodies which encompass the immunization and adsorption steps as disclosed above.

The term “specifically recognizing”, as used herein, is used in relation to polyclonal antibodies having affinity for, specificity for, and/or being specifically directed against a certain target, such as CPS and/or (wild-type) LOS of a specific capsular serotype. It does not exclude that the polyclonal antibody may also recognize non-CPS/LOS related low molecular weight structures of the LPS, preferably in a lesser amount as compared to non-absorbed, crude, anti-serum.

In particular embodiments, the polyclonal antibodies as taught herein allow to detect the CPS of C. canimorsus of the capsular serotype A, B, C, D or E as a high molecular weight (MW) band of at least 250 kDa in a protease-treated, preferably a proteinase K-treated, extract of C. canimorsus bacteria by Western blot.

In particular embodiments, the polyclonal antibodies as taught herein allow to detect (wild-type) LOS of C. canimorsus of the capsular serotype A, B, C, D or E as a high molecular weight (MW) band of at most 80 kDa, at most 70 kDa, at most 60 kDa, at most 50 kDa, at most 45 kDa, at most 40 kDa, at most 35 kDa, at most 30 kDa, at most 25 kDa, preferably at most 50 kDa and at least 10 kDa, at least 15 kDa, at least 20 kDa, preferably at least 15 kDa, in a protease-treated, preferably a proteinase K-treated, extract of C. canimorsus bacteria by Western blot.

Accordingly, the application also provides methods for serotyping C. canimorsus or identifying C. canimorsus as one of serotype A, serotype B, serotype C, serotype D or serotype E in a sample, which make use of serotype-specific antibodies. In these methods, and depending on the outcome envisaged or information required the sample may be contacted with one antibody against a given serotype or with different antibodies against the different serotypes. In further embodiments it may be of interest to determine whether the sample comprises a pathogenic capsular serotype of C. canimorsus (i.e. serotype A, B or C). In further embodiments, the method for serotyping C. canimorsus or identifying C. canimorsus as one of serotype A, serotype B, serotype C, serotype D or serotype E in a sample comprises detecting the presence of CPS/LOS of the C. canimorsus capsular serotype A, B, C, D and/or E in a sample or identifying C. canimorsus as one of serotype A, serotype B, serotype C, serotype D or serotype E through an immunoassay employing one or more polyclonal antibodies specifically recognizing CPS/LOS of the C. canimorsus capsular serotype A, B, C, D or E as taught herein. As the polyclonal antibodies obtained by the methods as described herein are able to recognize capsular serotype A, capsular serotype B, capsular serotype C, capsular serotype D or capsular serotype E of Capnocytophaga bacteria, other than C. canimorsus, it is preferably that, if determining whether or not other Capnocytophaga species are present in the sample is relevant, the specific identification of C. canimorsus bacteria is further confirmed using other methods.

In particular embodiments, the method for serotyping C. canimorsus or identifying C. canimorsus as one of serotype A, serotype B, serotype C, serotype D or serotype E in a sample comprises one or more of the following steps:
contacting a portion of said sample with a polyclonal antibody specifically recognizing CPS/LOS of the C. canimorsus capsular serotype A, as taught herein;
contacting a portion of said sample with a polyclonal antibody specifically recognizing CPS/LOS of the C. canimorsus capsular serotype B as taught herein;
contacting a portion of said sample with a polyclonal antibody specifically recognizing CPS/LOS of the C. canimorsus capsular serotype C as taught herein;
contacting a portion of said sample with a polyclonal antibody specifically recognizing CPS/LOS of the C. canimorsus capsular serotype D as taught herein; and/or
contacting a portion of said sample with a polyclonal antibody specifically recognizing CPS/LOS of the C. canimorsus capsular serotype E as taught herein; and the step of
detecting the presence of C. canimorsus capsular serotype A, B, C, D or E, or identifying C. canimorsus as one of serotype A, B, C, D or E through an immunoassay.

In particular embodiments, the method for serotyping C. canimorsus or identifying C. canimorsus as one of serotype A, serotype B, serotype C, serotype D or serotype E in a sample comprises one or more of the following steps:
contacting a portion of said sample with a polyclonal antibody obtained by adsorbing anti-serum obtained by immunization with (fixed) C. canimorsus 5 with (fixed) C. canimorsus 5 Y1C12 mutant bacteria and that specifically recognizes Capnocytophaga CPS/LOS of capsular serotype A, preferably C. canimorsus CPS/LOS of capsular serotype A, as taught herein;
contacting a portion of said sample with a polyclonal antibody obtained by adsorbing anti-serum obtained by immunization with (fixed) C. canimorsus 6 bacteria with an excess of (fixed) C. canimorsus 6 ΔwbuB mutant bacteria and that specifically recognizes CPS/LOS of Capnocytophaga capsular serotype B, preferably CPS/LOS of C. canimorsus capsular serotype B, as taught herein;
contacting a portion of said sample with a polyclonal antibody obtained by adsorbing anti-serum obtained by immunization with (fixed) C. canimorsus 9 with (fixed) C. canimorsus 9 ΔwbuB mutant bacteria and that specifically recognizes CPS/LOS of Capnocytophaga capsular serotype C, preferably CPS/LOS of C. canimorsus capsular serotype C, as taught herein;
contacting a portion of said sample with a polyclonal antibody obtained by adsorbing anti-serum obtained by immunization with (fixed) C. canimorsus 12 bacteria with (fixed) C. canimorsus 12 ΔwbtA mutant bacteria and that specifically recognizes CPS/LOS of Capnocytophaga capsular serotype D, preferably CPS/LOS of C. canimorsus capsular serotype D as taught herein; and/or
contacting a portion of said sample with a polyclonal antibody obtained by immunization with (fixed) C. canimorsus 4 bacteria with a mixture of (fixed) C. canimorsus bacteria comprising bacteria of C. canimorsus strains Cc1, Cc2, Cc3, Cc5, Cc6, Cc7, Cc8, Cc9, Cc10, Cc11, Cc12, Cc13, Cc14, Cc15, Cc16, Cc17, Cc18, Cc19, Cc20, Cc21, Cc22, Cc23, Cc24 and Cc25 and that specifically recognizes CPS/LOS of Capnocytophaga capsular serotype E, preferably CPS/LOS of C. canimorsus capsular serotype E as taught herein; and the step of
detecting the presence of C. canimorsus capsular serotype A, B, C, D or E or identifying C. canimorsus as one of serotype A, B, C, D or E through an immunoassay.

In a particular embodiment, the sample is treated with a broad spectrum protease enzyme to destroy all proteins, peptides and/or polypeptides in the sample before contacting said sample to the one or more polyclonal antibodies as taught herein. Preferably, said protease is proteinase K. The amount of proteins, peptides and/or polypeptides remaining in the sample is preferably at most 10%, at most 5%, at most 4%, at most 3%, at most 2%, at most 1%, more preferably less than 1% and may be assessed by methods known in the art.

In a particular embodiment, the sample is subjected to an immuno-purification step employing one or more polyclonal antibodies as described herein prior to or after the immunoassay for detecting the presence of C. canimorsus capsular serotype A, B, C, D or E or identifying C. canimorsus as one of serotype A, B, C, D or E. Preferably, immuno-purification step is performed prior to the immunoassay for detecting the presence of C. canimorsus capsular serotype A, B, C, D or E or identifying C. canimorsus as one of serotype A, B, C, D or E.

The term “immunoassay”, as used herein, refers to a biochemical test that measures the presence or concentration of a molecule, such as proteins or saccharides, in a solution through the use of an antibody or an antigen. Non-limiting examples of immunoassays are radioimmunoassay (RIA), enzyme immunoassay (EIA) such as ELISA, the latex agglutination method (LTIA), immunochromatography and Western blot analysis. In particular embodiments, immunopurification may also be used. The skilled person will understand that for detection by ELISA, the sample may need to be fixated to a substrate, for instance a 96 well plates, before the anti-serum can be added thereto. The polyclonal antibody will act as a mixture of primary antibodies. Subsequently, one or more labelled secondary antibodies directed against the mixture of primary antibodies are added. Examples of such labeling substances for secondary antibodies include enzymes such as horseradish peroxidase (HRP), alkaline phosphatase and beta-galactosidase; radioisotopes (RI), fluorescence substances such as FITC and tetramethylrhodamine thiocyanate; luminescence substrates such as chemiluminescence substrates; and visualizing substances such as colloidal gold and colored latex. The skilled person will understand that if the secondary antibody is a HRP, alkaline phosphatase or beta-galactosidase-coupled antibody, a substrate is required for detection.

If the detection is obtained by Western blot analysis, the skilled person will understand that the sample may need to be prepared for Western blot analysis (e.g. heat-treatment, proteinase K treatment) and loaded and electrophoretically separated on a gel suitable for Western blot analysis (e.g. a polyacrylamide gel). Next, the proteins, peptides, polypeptides and/or polysaccharide present on the gel may be transferred to a membrane (e.g. a nitrocellulose membrane) and said membrane may be blocked (e.g. with skimmed milk) prior to contacting said membrane to the polyclonal antibody, which will serve as a mixture of primary antibodies. Subsequently, one or more labelled secondary antibodies directed against the mixture of primary antibodies (e.g. HRP-coupled goat anti-rabbit polyclonal antibody, if the polyclonal antibody is obtained from rabbits) and optionally a substrate (e.g. chemiluminescent substrate) may be used for detection. The identification of a protein of interest in a Western blot relies among other factors on its molecular weight. To this end, the electrophoretically separated proteins in the sample are compared with a molecular weight standard of a sample known to comprise a C. canimorsus strain of the serotype A, B, C, D or E. Immunofluorescence and Western blot analysis may be performed by any method known by the skilled person. For fluorescence detection methods, large quantities of antigen are typically preferred. Accordingly, the sample is preferably obtained from microorganisms (particularly bacteria) isolated and/or propagated (e.g., cultured in vitro as known per se) from biological specimens. For all types of immunoassays, samples comprising non-capsulated and rough mutant C. canimorsus strains may be used as negative controls and samples comprising a C. canimorsus strain known to have either the capsular serotype A, B, C, D or E may be used as a positive control.

In a particular embodiment, the presence of C. canimorsus capsular serotype A is detected, when contacting a portion of the sample with a polyclonal antibody specifically recognizing CPS/LOS of the Capnocytophaga capsular serotype A, preferably CPS/LOS of the C. canimorsus capsular serotype A, as taught herein leads to a detectable interaction between said sample and said antibody, the presence of C. canimorsus capsular serotype B is detected, when contacting a portion of the sample with a polyclonal antibody specifically recognizing CPS/LOS of the Capnocytophaga capsular serotype B, preferably CPS/LOS of the C. canimorsus capsular serotype B, as taught herein leads to a detectable interaction between said sample and said antibody, the presence of C. canimorsus capsular serotype C is detected, when contacting a portion of the sample with a polyclonal antibody specifically recognizing CPS/LOS of the Capnocytophaga capsular serotype C, preferably CPS/LOS of the C. canimorsus capsular serotype C as taught herein leads to a detectable interaction between said sample and said antibody, the presence of C. canimorsus capsular serotype D is detected, when contacting a portion of the sample with a polyclonal antibody specifically recognizing CPS/LOS of the Capnocytophaga capsular serotype D, preferably CPS/LOS of the C. canimorsus capsular serotype D as taught herein leads to a detectable interaction between said sample and said antibody and the presence of C. canimorsus capsular serotype E is detected, when contacting a portion of the sample with a polyclonal antibody specifically recognizing CPS/LOS of the Capnocytophaga capsular serotype E, preferably CPS/LOS of the C. canimorsus capsular serotype E, as taught herein leads to a detectable interaction between said sample and said antibody. In a particular embodiment, said interaction between said sample and said antibody can be detected by immunoassays as described elsewhere herein.

In particular embodiments, the method for serotyping or the method for identifying C. canimorsus as one of serotype A, serotype B, serotype C, serotype D or serotype E as described herein further comprises a step of confirming that the detected bacteria are C. canimorsus bacteria by a C. canimorsus-specific PCR, preferably by C. canimorsus-specific 16S ribosomal DNA PCR as described elsewhere herein.

Also provided herein is are sets of polyclonal antibodies suitable for serotyping Capnocytophaga, preferably C. canimorsus, or identifying Capnocytophaga, preferably C. canimorsus, as one of serotype A, serotype B, serotype C, serotype D or serotype E. Serotyping using the polyclonal antibodies described herein can be performed by different methods which involve contacting the sample with one or more of the antibodies. For instance, it can be done through immunoassay involving at least one polyclonal antibody specifically recognizing the CPS/LOS of capsular serotype A, at least one polyclonal antibody specifically recognizing the CPS/LOS of capsular serotype B, at least one polyclonal antibody specifically recognizing the CPS/LOS of capsular serotype C, at least one polyclonal antibody specifically recognizing the CPS of capsular serotype D and at least one polyclonal antibody specifically recognizing the CPS/LOS of capsular serotype E.

Also provided herein is a kit of parts for detecting and/or serotyping Capnocytophaga, preferably C. canimorsus and/or or identifying Capnocytophaga, preferably C. canimorsus as one of serotype A, serotype B, serotype C, serotype D or serotype E comprising at least one polyclonal antibody specifically recognizing the CPS/LOS of capsular serotype A, at least one polyclonal antibody specifically recognizing the CPS/LOS of capsular serotype B, at least one polyclonal antibody specifically recognizing the CPS/LOS of capsular serotype C, at least one polyclonal antibody specifically recognizing the CPS/LOS of capsular serotype D and at least one polyclonal antibody specifically recognizing the CPS/LOS of capsular serotype E, and optionally further comprising reagents and/or carriers sufficient for performing the detection methods as taught herein.

The method for serotyping C. canimorsus, or identifying Capnocytophaga, preferably C. canimorsus, as one of serotype A, serotype B, serotype C, serotype D or serotype E in a sample by contacting said sample with one or more polyclonal antibodies as taught herein may be used to detect or quantify Capnocytophaga bacteria, preferably C. canimorsus bacteria, in a sample, for instance in a urine sample of a subject suspected to be infected with Capnocytophaga bacteria, preferably C. canimorsus bacteria.

The method for serotyping C. canimorsus or identifying C. canimorsus as one of serotype A, serotype B, serotype C, serotype D or serotype E in a sample by contacting said sample with one or more polyclonal antibodies as taught herein may be used to confirm the results of the method for serotyping C. canimorsus or identifying C. canimorsus as one of serotype A, serotype B, serotype C, serotype D or serotype E by polymerase-based nucleic acid amplification as taught herein. Likewise, the method for serotyping C. canimorsus or identifying C. canimorsus as one of serotype A, serotype B, serotype C, serotype D or serotype E by polymerase-based nucleic acid amplification as taught herein may be used to confirm the results of the method for serotyping C. canimorsus or identifying C. canimorsus as one of serotype A, serotype B, serotype C, serotype D or serotype E in a sample by contacting said sample with one or more polyclonal antibodies as taught herein.

Also provided herein is a method for detecting the presence of C. canimorsus of serotype A, serotype B, serotype C, serotype D or serotype E in a sample, said method comprising a step of subjecting said sample to an immuno-purification step employing one or more polyclonal antibodies as described herein specific for a given serotype; and a step of identifying in the immuno-purified sample so obtained C. canimorsus bacteria by performing a C. canimorsus-specific PCR, preferably a C. canimorsus-specific 16S ribosomal DNA PCR. C. canimorsus infections may be prevented by vaccination. For instance, C. canimorsus infections may be prevented by vaccination of persons that come into contact with animals such as dogs or cats (i.e. veterinarians, professionals working with dogs or disabled individuals assisted by an assistance dog), especially when these persons have a higher risk of C. canimorsus infection. For instance, splenectomy and alcohol abuse are common predisposing factors. Provided herein are polyvalent vaccines of two, three, four or five different C. canimorsus capsules which protect against the identified dangerous C. canimorsus strains. Such vaccines overcome the need of having to develop a polyvalent vaccine of all identified (most dangerous) C. canimorsus strains.

Accordingly, a further aspect is a polyvalent vaccine against C. canimorsus, or for protection against an infection with C. canimorsus, comprising
inactivated or attenuated cells of a C. canimorsus strain of the serotype A selected from the list consisting of C. canimorsus strains Cc1, Cc2, Cc3, Cc5, Cc10, Cc13, Cc15, Cc21, Cc22, Cc24 and Cc25, or fragments thereof;
inactivated or attenuated cells of a C. canimorsus strain of the serotype B selected from the list consisting of C. canimorsus strains Cc6, Cc8, Cc11, Cc16, Cc17, Cc18 and Cc23, or fragments thereof;
inactivated or attenuated cells of a C. canimorsus strain of the serotype C selected from the list consisting of C. canimorsus strains Cc9, Cc14, Cc19 and Cc20, or fragments thereof;
inactivated or attenuated cells of a C. canimorsus strain of the serotype D selected from the list consisting of C. canimorsus strains Cc12 and Cc7, or fragments thereof; and/or
inactivated or attenuated cells of a C. canimorsus strain of the serotype E, wherein said C. canimorsus strain is C. canimorsus strain Cc4, or fragments thereof.

The term “polyvalent” or “multivalent”, as used herein, in the combination with the term “vaccine” refers to a vaccine prepared from cultures or antigens of more than one strain of C. canimorsus. The valency of a polyvalent vaccine may be denoted with a Greek or Latin prefix (e.g., tetravalent or quadrivalent). The vaccine may be a preventive (i.e. prophylactic) A preventive vaccine may be administered to a subject (animal or human) that is free of the targeted infection in order to produce antibodies to pathogenic C. canimorsus bacteria, which are able to recognize and destroy the pathogenic C. canimorsus bacteria that might enter the subject at a later time.

In certain preferred embodiments of the methods or uses as taught herein, the subject is a human subject. In other embodiments, the subject a canine or a feline, preferably a canine, such as a dog. In particular embodiments, in the context of non-human animals used for immunization, the animal is a rodent or farm-animal (cattle or pig).

The terms “prevent” or “prevention”, as used herein, encompass both the delaying or averting a disease or condition (such as bacterial infection) that has not developed yet. However, there are preferably indications that the disease or condition would occur without administering the vaccine.

The term “inactive”, as used herein, refers to killed micro-organisms.

The term “attenuated”, as used herein, refers to micro-organisms prepared from live microorganisms cultured under adverse conditions, leading to loss of their virulence but retention of their ability to induce protective immunity.

In a particular embodiment, the polyvalent vaccine for protection against an infection C. canimorsus comprises inactivated or attenuated cells of at least two C. canimorsus strains of at least two capsular serotypes selected from serotype A, serotype B, serotype C, serotype D or serotype E or derivatives thereof. It is believed that given that different Capnocytophaga have the same capsular serotypes, vaccination with a C. canimorsus strain of a given capsular serotype will also protect against infection with other Capnocytophaga strains of that same capsular serotype.

In particular embodiments, the polyvalent vaccine against C. canimorsus, or for protection against an infection with C. canimorsus as described herein comprises:
inactivated or attenuated cells of a C. canimorsus strain of the serotype A selected from the list consisting of C. canimorsus strains Cc1, Cc2, Cc3, Cc5, Cc10, Cc13, Cc15, Cc21, Cc22, Cc24 and Cc25, or fragments thereof;
inactivated or attenuated cells of a C. canimorsus strain of the serotype B selected from the list consisting of C. canimorsus strains Cc6, Cc8, Cc11, Cc16, Cc17, Cc18 and Cc23, or fragments thereof;
inactivated or attenuated cells of a C. canimorsus strain of the serotype C selected from the list consisting of C. canimorsus strains Cc9, Cc14, Cc19 and Cc20, or fragments thereof;
inactivated or attenuated cells of a C. canimorsus strain of the serotype D selected from the list consisting of C. canimorsus strains Cc12 and Cc7, or fragments thereof; and
inactivated or attenuated cells of a C. canimorsus strain of the serotype E, wherein said C. canimorsus strain is C. canimorsus strain Cc4, or fragments thereof.

C. canimorsus capsular serotypes A, B and C were found to be more virulent for humans than C. canimorsus strains from other C. canimorsus capsular serotypes. Accordingly, polyvalent vaccines protecting against C. canimorsus capsular serotypes A, B and/or C would provide a good protection against a C. canimorsus infection.

Therefore, in a particular embodiment, the polyvalent vaccine for protection against an infection C. canimorsus comprises inactivated or attenuated cells of a C. canimorsus strain of the C. canimorsus capsular serotypes A, B and C, or derivatives thereof.

In particular embodiments, the polyvalent vaccine against C. canimorsus, or for protection against an infection with C. canimorsus as described herein comprises:
inactivated or attenuated cells of a C. canimorsus strain of the serotype A selected from the list consisting of C. canimorsus strains Cc1, Cc2, Cc3, Cc5, Cc10, Cc13, Cc15, Cc21, Cc22, Cc24 and Cc25, or fragments thereof;
inactivated or attenuated cells of a C. canimorsus strain of the serotype B selected from the list consisting of C. canimorsus strains Cc6, Cc8, Cc11, Cc16, Cc17, Cc18 and Cc23, or fragments thereof; and
   inactivated or attenuated cells of a C. canimorsus strain of the serotype C selected from the list consisting of C. canimorsus strains Cc9, Cc14, Cc19 and Cc20, or fragments thereof. Bacterial infection in a subject with a pathogenic C. canimorsus may be prevented by administering the polyvalent vaccine as described herein to a subject free of the targeted infection, for example a human or animal subject at risk of being infected.

For instance, administering the polyvalent vaccine as described herein to animals, such as canines or felines, more particularly a dog or cat, can be used to prevent bacterial infection with a pathogenic C. canimorsus in humans that are in contact therewith. Accordingly, the invention can be used for preventing a C. canimorsus infection, most particularly an infection with a pathogenic C. canimorsus in a non-human animal, preferably a canine or feline, even when such infection would not be pathogenic for said non-human animal.

In particular embodiments, the vaccine is used as a therapeutic vaccine. A therapeutic vaccine is administered to a subject infected with the targeted C. canimorsus bacteria. More particularly for humans, certain pathogenic C. canimorsus bacteria are pathogenic. In cases where naturally produced antibodies are ineffective, the administration of therapeutic vaccines can be of interest. In other words, therapeutic vaccines aim to strengthen the natural immune response against the C. canimorsus.

Similarly, administering the polyvalent vaccine as described herein to animals carrying or suspected to be carrying the (for humans) pathogenic C. canimorsus, such as canines or felines, more particularly a dog or cat, is envisaged. This can be used to prevent bacterial infection with a pathogenic C. canimorsus in humans that are in contact therewith. Accordingly, the invention can be used for eliminating a C. canimorsus infection, most particularly an infection with a pathogenic C. canimorsus from a non-human animal, preferably a canine or feline, even when such infection is not pathogenic for said non-human animal.

The polyvalent vaccine as described herein may be administered to the subject intradermally, subcutaneously, or intramuscularly.

Another aspect provides in a method of preparing the polyvalent vaccine as described herein.

Methods of preparing polyvalent vaccines are known by the skilled person. Such methods may comprise the steps of separately propagating the selected C. canimorsus strains (e.g. in bioreactors), treating the respective bacteria in such a way that they are either rendered inactive or attenuated, and combining the inactivated or attenuated bacteria to produce a polyvalent vaccine containing at least two different C. canimorsus strains of at least two capsular serotypes. Adjuvants for enhancing the immune response of the antigens, stabilizers for increasing the storage life, and preservatives allowing the use of multidose vials may be added as needed.

The present invention is further illustrated in the following non-limiting examples.

### EXAMPLES

### Material and Methods

### Bacterial Strains and Culture Conditions

C. canimorsus strains isolated from patients used in this study are Cc1 (BCCM/LMG 11511; CCUG 17234; strain P810; strain SSI P810), Cc2, Cc3, Cc4, Cc5 (BCCM/LMG 28512), Cc6, Cc7, Cc8, Cc9 (BCCM/LMG 11510, CCUG 12569, CDC A3626), Cc10 (BCCM/LMG 11541, CCUG 24741, ATCC 35978, CDC C8936), Cc11 (BCCM/LMG 11551, MCCM 01373), Cc12 (ATCC 35979, CDC 7120, CCUG 53895), Cc13, Cc14, Cc15, Cc16, Cc17, Cc18, Cc19, Cc20 (CCUG 55909), Cc21 (CCUG 60839), Cc22 (CCUG 20318), Cc23 (CCUG 48899), Cc24 (CCUG 67384) and Cc25 (CCUG 66222). C. canimorsus strains isolated from dog mouth used in this study are CcD3, CcD5, CcD6, CcD10, CcD13, CcD16, CcD18, CcD20, CcD25, CcD33, CcD34, CcD35, CcD37, CcD39, CcD40, CcD43, CcD44, CcD47, CcD51, CcD52, CcD53, CcD57, CcD58, CcD63, CcD68, CcD69, CcD71, CcD73, CcD80, CcD81, CcD84, CcD89, CcD96, CcD101, CcD104, CcD105, CcD106, CcD113, CcD115, CcD116, CcD117, CcD118, CcD119, CcD120, CcD122, CcD124, CcD126, CcD129, CcD130 and CcD131 as previously described in Renzi F. et al. (Renzi F. et al., Only a subset of C. canimorsus strains is dangerous for humans. Emerg Microbes Infect, 2016, 5:e29) and CcD76 and CcD77 as previously described in Manfredi P. et al. (Manfredi P. et al., New iron acquisition system in Bacteroidetes, Infect Immun, 2015, 83(1):300-310). C. canimorsus were grown on heart infusion agar (HIA; BD Difco, Franklin Lakes, N.J., USA) supplemented with 5% sheep blood (SB; Oxoid, Basingstoke, UK) plates (SB plates) for 48 h at 37° C. with 5% CO₂. Escherichia coli were routinely grown in lysogeny broth (LB; Invitrogen, Waltham, Mass., USA) at 37° C. Antibiotics used as selective agents were added at the following concentrations: 100 μg/mL ampicillin (Amp) and 50 μg/mL kanamycin (Kan) for E. coli and 20 μg/mL gentamicin (Gm), 10 μg/mL erythromycin (Em) and 10 μg/mL cefoxitin (Cfx) for C. canimorsus. Unless otherwise stated products were purchased from Sigma-Aldrich (Darmstadt, Germany).

### Anti-Sera Production and Adsorption

### Anti-Sera Production

Wild-type C. canimorsus bacteria producing the relevant CPS/LOS were grown for 2 days on sheep blood (SB) plates supplemented with gentamicin, gently scraped from the agar, resuspended and washed in PBS. Bacteria were fixed overnight in 0.3% paraformaldehyde (PFA) and then washed in PBS. To generate polyclonal sera 5.10⁸ fixed bacteria were inoculated subcutaneously with complete Freund's adjuvant to a rabbit. Subsequently booster injections of 5.10⁸ fixed bacteria with incomplete Freund's adjuvant were administered to the rabbit. Blood was collected after at least one boost and centrifuged to retrieve the serum, which contained the antibodies raised against the fixed bacteria. Sera were generated at the University of Namur (Belgium) or at the Centre d'économie rurale Groupe (CER Groupe; Aye, Belgium). The respective Animal Welfare Committees approved the animal handling and procedures.

### Anti-Sera Adsorption

The crude anti-sera which were produced using fixed-bacteria contained antibodies recognizing all the immunogenic structures exposed at the outer membrane of the bacteria, including the CPS/LOS but also proteins and lipoproteins (Manfredi, P. et al., Mol Microbiol, 2011). To deplete the antibodies recognizing structures other than the CPS/LOS the anti-sera were “adsorbed” using a rough (no or at least an incomplete LOS) and non-capsulated mutant. For this, the mutant strain were grown for 2 days on SB plates supplemented with gentamicin and erythromycin gently scraped from the agar, resuspended, washed in PBS and fixed overnight in 4% PFA. Fixed bacteria were washed, pelleted and resuspended in the anti-serum to be adsorbed, on a rotating wheel at room temperature (RT) for two hours. During this incubation antibodies recognizing any other epitopes than the ones present in the CPS/LOS, bind to the mutant bacteria. Bacteria along with the bound antibodies are removed from the serum by repeated centrifugations. To ensure that only the anti-CPS/LOS antibodies remain in the treated anti-serum, this incubation was repeated 3 times. To make the specific anti-A serum, the full anti-Cc5 serum was adsorbed with Cc5 Y1C12 mutant, which is non-capsulated and present an incomplete LOS (Renzi F. et al, Scientific Reports, 2016). To make the specific anti-B serum, the crude anti-Cc6 serum was adsorbed with Cc6 ΔwbuB bacteria. To make the specific anti-C serum, the crude anti-Cc9 serum was adsorbed with Cc9 ΔwbuB bacteria. To make the specific anti-D serum, the crude anti-Cc12 serum was adsorbed with Cc12 ΔwbtA bacteria. To make the specific anti-E serum, the crude anti-Cc4 serum was adsorbed with a mixture of 24 wild-type clinical strains (all belonging to a different serotype).

### Specificity of the Adsorbed Anti-Serum

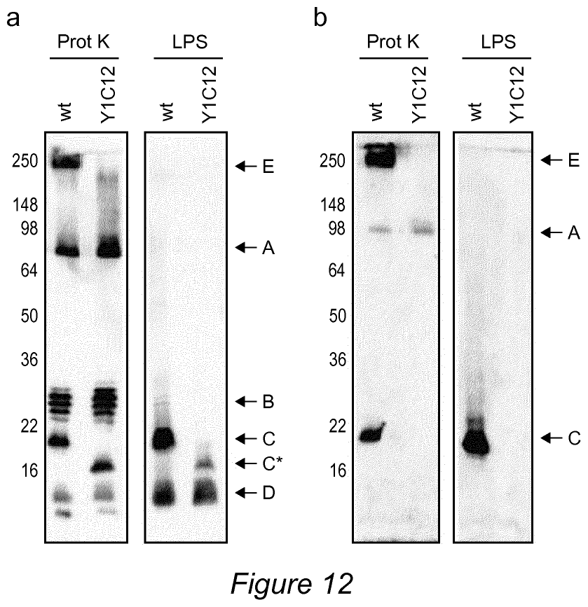

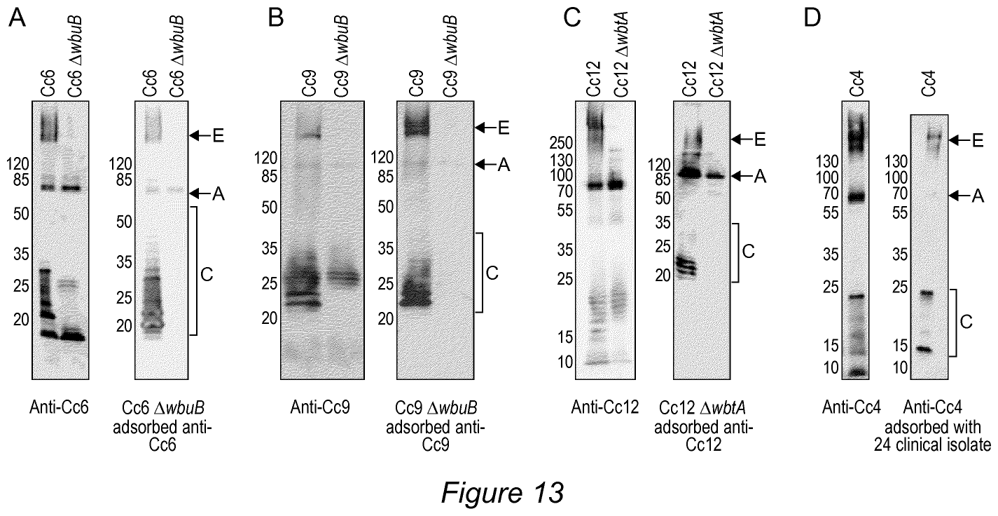

Anti-serum production and adsorption has been previously used and validated by western blotting of polysaccharide structures (Renzi, F. et al., Scientific Reports, 2016; Shin, H. et al., Infect Immun, 2009). To control the adsorption of the anti-Cc5 serum with the Y1C12 mutant, western blots were done on proteinase K-treated lysates of WT and Y1C12 bacteria using the crude anti-Cc5 or the Y1C12-adsorbed anti-Cc5 serum (FIG. 12). While the crude serum recognized the CPS (band E), LOS (band C) and mutant LOS (band C*), it recognized also other uncharacterized structures (band A, B and D). The adsorbed serum only recognized the CPS and the WT LOS. Antibodies recognizing the band A were still present after the adsorption but in reduced amount as compared to anti-CPS/LOS antibodies. Adsorption was also validated by western blots for the following sera: Cc6 ΔwbuB adsorbed anti-Cc6, Cc9 ΔwbuB adsorbed anti-Cc9, Cc12 ΔwbtA adsorbed anti-Cc12 and anti-Cc4 adsorbed with 24 wild-type clinical strains (FIG. 13).

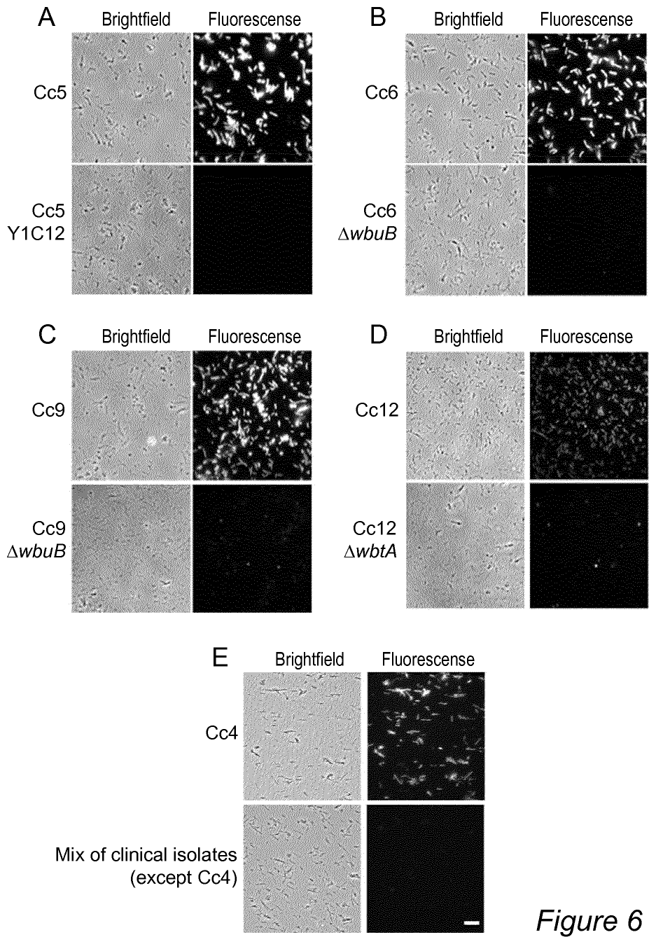

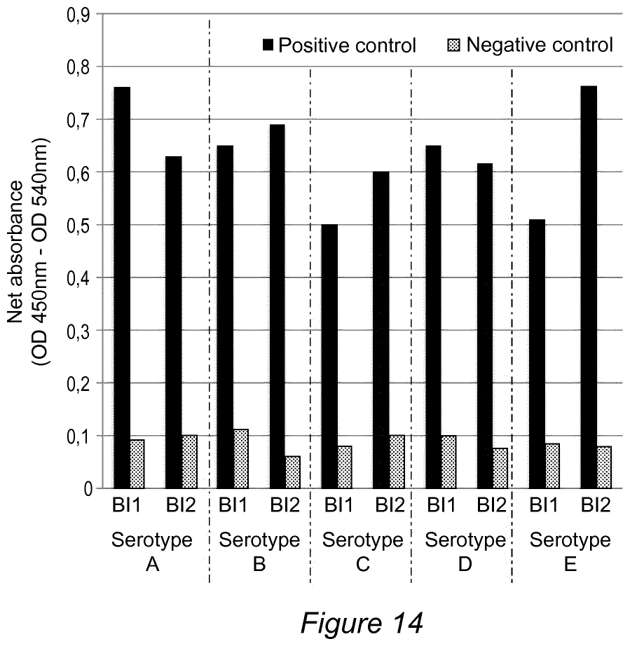

The efficacy of the adsorption, and thus the specificity of the adsorbed serum, was also validated, for each serum, by immunofluorescence staining and microscopy analysis. Briefly, 300 μl of each bacterial suspension (adjusted to an OD₆₀₀ of 0.25) was incubated on poly-D-lysine-coated glass coverslips for 30 minutes at 37° C. Coverslips were washed and fixed with 4% PFA for 15 min. Coverslips were washed again and blocked with 1% bovine serum albumin (BSA) for 1 hour at RT. Bacteria were stained with the adsorbed sera ( 1/1000 in PBS) for 1 hour at RT followed by an incubation with a fluorochrome-conjugated anti-rabbit IgG antibody (alexa fluor 488-coupled donkey anti-rabbit antibody; 1/5000 in PBS; Life technologies, Waltham, Mass., USA; or a Texas Red coupled goat anti-rabbit antibody; 1/1000 in PBS, Southern Biotech, Birmingham, Ala., USA). Coverslips were mounted using mowiol, images were acquired with an Axio Imager. Z1 (Zeiss, Oberkochen, Germany) and samples were then analyzed by microscopy (Zen 2012 software, Zeiss) (FIG. 6). While the adsorbed serum recognized the WT strain against which it was raised, it no longer recognized the mutant strain used to adsorb it. In the case of the anti-Cc4 serum, the adsorbed anti-Cc4 serum did not recognize the mix of the 24 clinical isolates used to adsorb it. This experiment confirmed that the antibodies recognizing epitopes other than the ones found in the CPS/LOS have been successfully depleted from the anti-serum. The adsorbed anti-sera thus specifically recognize the CPS/LOS from the strain against which it has been raised. In addition, the Applicants also checked the specificity of the adsorbed anti-sera by ELISA using the protocol as described below (FIG. 14). Two different rabbit bleeding were tested for all serotypes. While a signal comprised between 0.5 and 0.7 was observed when the adsorbed sera was tested against the type strain, a value equal or lower than 0.1 was found when the rough and non-capsulated mutants were used. For the serotype E, the negative control refers to a mix of the 24 clinical strains (except Cc4).

### Mutagenesis by Allelic Exchange

Mutagenesis of Cc6, Cc9 and Cc12 strains was performed as previously described Mally et al. (Mally et al., 2008, Applied and Environmental Microbiology). The C. canimorsus deletion mutants used are Y1C12 (C. canimorsus 5 Ccan_23370::Tn4351; Em^{r} as described in Shin et al., Resistance of C. Canimorsus to killing by human complement and polymorphonuclear leukocytes, Infect Immun, 2009, 77(6):2262-2271.), CC6ΔwbuB (i.e. the replacement of Cc6_1430029 in C. canimorsus 6 by ermF using primers 7958, 7959, 7962, 7975, 7961, 7974; Em^{r}); Cc9 ΔwbuB (i.e. the replacement of CC9_740038 in C. canimorsus 9 by ermF using primers 7958, 7959, 7960, 7961, 7962, 7963; Em^{r}) and Cc12 ΔwbtA (i.e. the replacement of CCAN12_760057 in C. canimorsus 12 by ermF using primers 8063, 8064, 8065, 8066, 8067, 8068; Em^{r}). The E. coli strains used are Top10 (F⁻ mcrA Δ(mrr-hsdRMS-mcrBC) ϕ80lacZΔM15 ΔlacX74 recA1 araD139 Δ(araleu)7697 galU galK rpsL endA1 nupG; Smr; Invitrogen) and S17-1 (hsdR17 recA1 RP4-2-tet::Mulkan::Tn7; Smr; as described in Simon R., et al., A broad host range mobilization system for in vivo genetic engineering: transposon mutagenesis in gram negative bacteria, Nat Biotech, 1983, 1(9):784-791). Briefly, replacement cassettes with flanking regions spanning approximately 500 base pairs (bp) homologous to regions directly framing targeted genes were constructed with a three-fragment overlapping PCR strategy. First, two PCRs were performed on 100 ng of Cc6, Cc9 or Cc12 genomic DNA with primers 1.1 and 1.2 for the upstream flanking regions and with primers 2.1 and 2.2 for the downstream regions. Primers 1.2 and 2.1 contained an additional 5′ 20-nucleotide extension homologous to the ermF insertion cassette. The ermF resistance cassettes were amplified from plasmids pMM13 (Mally et al., 2008, Applied and Environmental Microbiology) DNA, with primers 3.1 and 3.2. All three PCR products were cleaned and then mixed in equal amounts for PCR using Phusion polymerase (Finnzymes, Espoo, Finland). The initial denaturation was at 98° C. for 2 min, followed by 12 cycles without primers to allow annealing and elongation of the overlapping fragments (1 cycle consists of 98° C. for 30 s, 50° C. for 40 s, and 72° C. for 2 min). After the addition of external primers (primers 1.1 and 2.2), the program was continued with 20 cycles (1 cycle consists of 98° C. for 30 s, 50° C. for 40 s, and 72° C. for 2 min 30 s) and finally 10 min at 72° C. Final PCR products consisting of locus::ermF insertion cassettes were then digested with PstI and SpeI (New Englands Biolabs, Ipswich, Mass., USA) for cloning into the appropriate sites of the C. canimorsus suicide vector pMM25 (Mally et al., 2008, Applied and Environmental Microbiology). The resulting plasmids were transferred by RP4-mediated conjugative DNA transfer from E. coli S17-1 to the corresponding C. canimorsus strains to allow integration of the insertion cassette. Transconjugants were then selected for the presence of the ermF cassette on erythromycin-containing plates and checked for sensitivity to cefoxitin. Deletion of the appropriate regions was verified by PCR. Oligonucleotides used in this study are provided in Table 7 (FIG. 11).

### Western Blotting of Polysaccharide Structures

Bacteria were harvested by gently scraping colonies off the agar surface of Gm SB plate and resuspended in PBS. Bacteria suspensions were adjusted to an OD₆₀₀ of 1 in PBS. 750 μL of the suspension were pelleted and resuspended in 125 μL loading buffer (1% sodium dodecyl sulphate (SDS), 10% glycerol, 50 mM dithiothreitol, 0.02% bromophenol blue, 45 mM Tris (pH6.8)). Samples were heated for 10 minutes at 99° C. Proteinase K (VWR Chemicals, Radnor, Pa., USA), was added to a final concentration of 50 μg/mL and samples were incubated overnight at 37° C. Subsequently, samples were heated for 10 minutes at 99° C. and proteinase K was added again at the same final concentration. Samples were incubated for 3 hours at 55° C., heated for 5 minutes at 99° C. and loaded on a 12% polyacrylamide gel. After SDS-PAGE (polyacrylamide gel electrophoresis), proteinase K resistant structures were transferred on a nitrocellulose membrane (GE Healthcare, Chicago, Ill., USA). Membranes were blocked with non-fat dry milk and incubated with polyclonal crude or adsorbed sera (dilutions ranging from 1/400 to 1/8000) followed by incubation with a horseradish peroxidase (HRP)-coupled goat anti-rabbit polyclonal antibody ( 1/2000; Dako Agilent Technologies, Santa Clara, Calif., USA). Membranes were revealed using a chemiluminescent substrate (KLP, Gaithersburg, Md., USA) on an Amersham Imager 600 (GE Healthcare). All incubations were conducted in 5% non-fat dry milk diluted in PBS 0.05% Tween.

### ELISA Screening for CPS

Bacteria suspensions were adjusted to an OD₆₀₀ of 0.5 and were killed by an incubation of 30 minutes at 70° C. Heat-killed bacteria suspensions were used to coat 96 well plates (ThermoScientific, Waltham, Mass., USA) overnight at 4° C. The next day plates were washed to remove unfixed bacteria and blocked for 1 hour at RT with 1% BSA in PBS. Plates were washed and incubated with adsorbed polyclonal serum ( 1/1000 to 1/5000 in PBS) for 1 hour at RT. Plates were washed again and incubated with HRP-coupled goat anti-rabbit polyclonal antibody for 1 hour at RT (Dako Agilent Technologies; 1/2000 in PBS). Plates were then washed and revealed using 3,3′,5,5′-Tetramethylbenzidine (TMB) as a chromogenic substrate.

### PCR Screening of CPS Serotypes

Bacteria were grown on SB plates supplemented with Gm and a single colony was resuspended in 100 μL ddH₂O and heated for 15 minutes at 98° C. Two microliters were used as template for amplification. PCR detection was performed using the Promega Go Taq® G2 polymerase (Madison, Wis., USA) under the following conditions: an initial denaturation at 95° C. for 4 min, followed by 35 cycles of denaturation at 95° C. for 30 s, annealing at 52° C. for 45 s, extension at 72° C. for 1 min and 30 s, and a final extension at 72° C. for 7 min. Oligonucleotides used in this study correspond to those provided in Table 4.

### Synteny Analysis

Synteny statistics were obtained using the MicroScope PkGDB synteny statistics tool (https://www.genoscope.cns.filagc/microscope/home/index.php) (Vallenet et al., 2006, Nucleic Acids Res). Putative orthologous relations based on the bi-directional best hit (BBH) criterion were considered for at least 35% of sequence identity on 80% of the length of the smallest protein. For the synteny analysis, all possible kinds of chromosomal rearrangements are allowed (inversion, insertion/deletion) and the gap parameter, representing the maximum number of consecutive genes which are not involved in a synteny group, is set to five genes.

### Statistical Analysis

Statistical significance was evaluated by Fisher's exact tests using the BiostaTGV website (https://marne.u707.jussieu.fr/biostatgv).

### Example 1. Capsular Serotyping Identifies 5 Serotypes in a Collection of C. canimorsus Strains Isolated from Clinical Cases

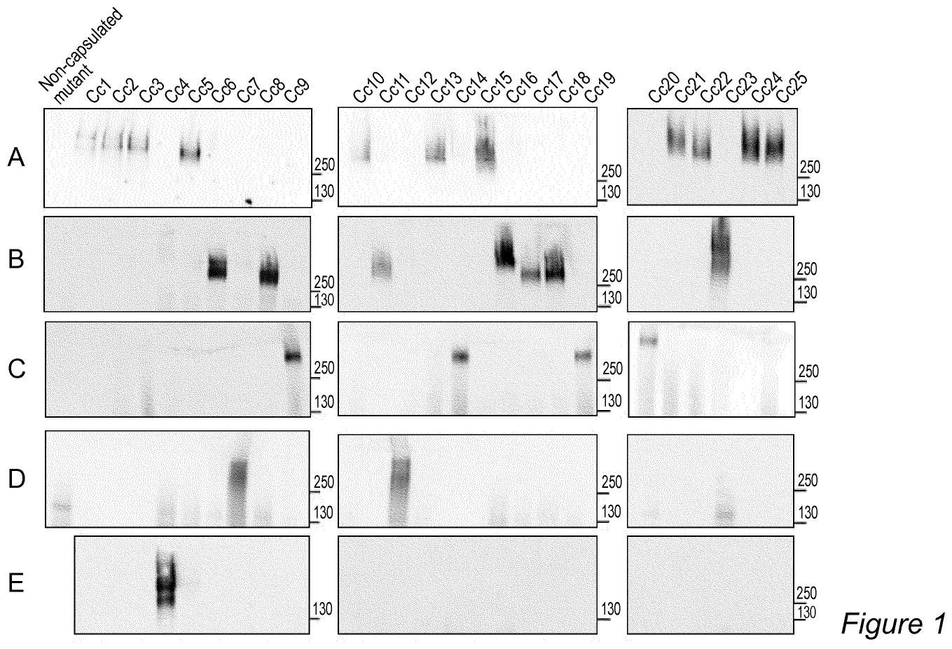

The prevalence of the capsular serotype of type strain Cc5 was tested in a collection of 25 C. canimorsus strains isolated from human infections (see materials and methods section above). Whole bacteria were digested with proteinase K and bacterial polysaccharides were analyzed by Western blot using an anti-serum directed against Cc5 bacteria and adsorbed with the non-capsulated Cc5 transposon mutant Y1C12 (Renzi F. et al., Scientific Reports, 2016; Shin H. et al., 2009, Infect Immun). In addition to Cc5, the serum recognized a high molecular weight (MW) band (>250 kDa) in the extracts from ten strains, namely Cc1, Cc2, Cc3, Cc10, Cc13, Cc15, Cc21, Cc22, Cc24 and Cc25 (FIG. 1 a). Since this band was identified as the CPS of Cc5 (Renzi F. et al., Scientific Reports, 2016), Applicants concluded that the capsular serotype of Cc5 was shared with these 10 strains representing 44% of the collection of strains isolated from patients. The Applicants named this capsular serotype A.

To determine the capsular serotypes of the 14 clinical strains not reacting with the Y1C12-adsorbed anti-Cc5 serum, 9 new anti-sera were raised and tested by Western blot on polysaccharide extracts from these 14 strains. Antisera raised against Cc6, Cc9, Cc12 (ATCC 35979) and Cc4 allowed detecting a high MW polysaccharide, most likely corresponding to a CPS (FIG. 1) in all the 14 strains. The anti-Cc6 serum recognized a high MW polysaccharide structure in Cc6 but also in Cc8, Cc11, Cc16, Cc17, Cc18 and Cc23 (FIG. 1B). This serotype, named B, had thus a prevalence of 28% in the collection of strains isolated from clinical cases, with 7 strains positive out of 25. The anti-Cc9 serum recognized a high MW polysaccharide structure in Cc9, Cc14, Cc19 and Cc20 (FIG. 10). This serotype was named C and had a prevalence of 16%. The anti-Cc12 serum recognized a high MW polysaccharide structure in Cc12 and Cc7 (FIG. 1D). The prevalence of this serotype, named D, was thus more limited than A, B and C serotypes, and was of 8%. Finally, the anti-Cc4 serum recognized a high MW polysaccharide band only in Cc4. This serotype had thus a prevalence of only 4% and was named E (FIG. 1E).

In order to confirm that the high MW bands recognized are CPS, the Applicants next attempted to generate non-capsulated deletion mutants of Cc6, Cc9, Cc12 and Cc4. To this aim the Applicants sequenced the genomes of the strains Cc6, Cc9 and Cc4 and used the previously published draft genome of Cc12 (Manfredi P., 2015, Genome Announc). Homologs of Cc5 wbuB gene (Ccan_23370), which is the gene mutated in the non-capsulated Y1C12 mutant and encodes a N-acetyl-fucosamine (FucNAc) transferase (Zahringer U., 2014, J Biol Chem) were found in the genomes of Cc6 (Cc6_1430029) and Cc9 (CC9_740038) but not in those of Cc12 and Cc4. In the latter genomes the Applicants identified homologs of Cc5 wbtA (Ccan_23400) that is mutated in the capsular mutant Y1D1 (Renzi F. et al., Scientific Reports, 2016) of Cc5 and putatively encode a UDP-N-acetylglucosamine 4,6-dehydratase (CCAN12_760057 and CC4_530070 respectively) (Renzi F. et al., Scientific Reports, 2016). The wbuB genes were thus mutated in Cc6 and Cc9 while gene wbtA was mutated in Cc12. Gene wbtA from Cc4 could not be mutated despite several attempts. The polysaccharide extracts from the mutants of Cc6, Cc9 and Cc12 did not react with the anti-Cc6, anti-Cc9 and anti-Cc12 sera, indicating that the high MW band was indeed a CPS (FIGS. 1B, C and D). For Cc4, the Applicants have no direct evidence that the high MW polysaccharide is a CPS.

The Applicants thus conclude that the C. canimorsus strains isolated from human infections are, like strain Cc5, endowed with a CPS and that the antigenic repertoire of these CPS is limited since serotypes A, B and C covered the capsules of 88% of the strains from the collection (22 out of 25 strains).

### Example 2. Capsular Serotypes A, B and C are not Linked to a Geographic Area

The Applicants next assessed whether these capsular serotypes were not linked to the geographical origin of the strains. Among the strains analyzed, ten originated from Belgium, four from Sweden, three from the USA, three from Switzerland, one from France, one from Germany, one from the Netherlands, one from the UK and finally one from Denmark. The 11 strains belonging to the capsular serotype A were isolated in 6 different countries: Sweden (4/11; i.e. Cc1, Cc21, Cc24 and Cc25), Belgium (3/11; i.e. Cc2, Cc3 and Cc5), USA (1/11; i.e. Cc10), the Netherlands (1/11, i.e. Cc13), Denmark (1/11; i.e. Cc22) and Switzerland (1/11; i.e. Cc15). The serotype B strains also originated from three different countries: Belgium (5/7; i.e. Cc6, Cc8, Cc16, Cc17 and Cc18), Switzerland (1/7; i.e. Cc11) and France (1/7; i.e. Cc23). Serotype C strains also came from four different countries (USA (i.e. Cc9), Germany (i.e. Cc14), Belgium (i.e. Cc19) and UK (i.e. Cc20)). Finally, the capsular serotype D was isolated in Belgium (i.e. Cc7) and in the USA (i.e. Cc12) and the capsular serotype E was isolated in Switzerland (i.e. Cc4). Hence, no apparent geographical bias was observed in the capsular serotypes distribution with at least 3 countries being represented for the dominant serotypes A, B and C.

### Example 3. Enrichment of Capsular Serotype A, B and C Among the Strains Isolated from Human Infections

The Applicants next assessed the prevalence of the capsular serotypes A to E among a collection of 52 strains isolated from dogs (see Materials and methods section above). To test this collection the Applicants set up an ELISA screening using entire heat-killed bacteria. To this aim, the Applicants needed sera that were specifically recognizing the CPS. For serotype A, the Applicants took the Y1C12-adsorbed anti-Cc5 serum; for serotypes B, C and D, the Applicants adsorbed the crude anti-Cc6, anti-Cc9 and anti-Cc12 sera with Cc6 wbuB, Cc9 wbuB and Cc12 wbtA mutant bacteria respectively. The efficacy of the different adsorptions was validated by immunofluorescence staining and microscopy analysis. While the adsorbed serum recognized the WT strain against which it was raised, it no longer recognized the mutant strain used to adsorb it (FIG. 6). As the Applicants did not succeed to generate the non-capsulated Cc4 mutant required to adsorb the anti-Cc4 serum (serotype E), the Applicants adsorbed the anti-Cc4 serum with the 24 other human isolates that were shown not to share the same capsular epitopes (FIG. 1E). The adsorption of this serum was also validated by immunofluorescence and microscopy analysis, using Cc4 as a positive control and a mix of the 24 other clinical strains as a negative control (FIG. 6).

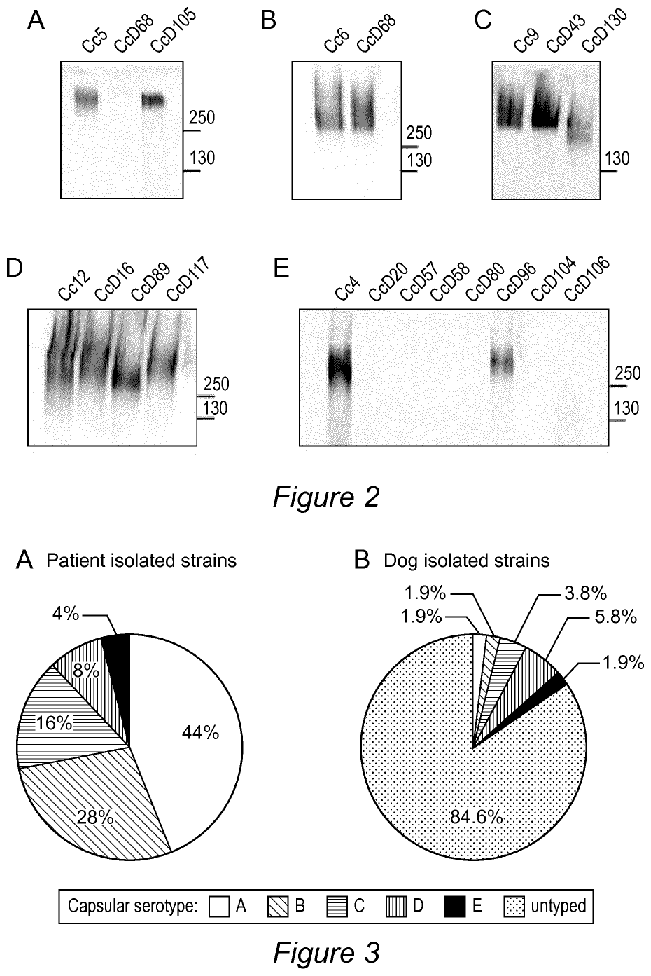

The five adsorbed sera were then used to test the collection of dog-isolated strains by ELISA. The reactivity of each strain was calculated with respect to the value of the type strain of each serotype (Cc5 for A, Cc6 for B, Cc9 for C, Cc12 for D and Cc4 for E). The non-capsulated mutant strains used to adsorb the sera were used as negative controls. The results of the screening are summarized in Table 5. Only two strains, namely CcD68 and CcD105 were positive for serotype A with a reactivity of 43%±7 and 107%±28 respectively. The two strains were tested by a Western blot analysis of the proteinase K-treated lysates and only the strongly reacting CcD105 displayed a serotype A capsule (FIG. 2A). For serotype B, only strain CcD68 was found to be positive (110%±11), and this was confirmed by a Western blot analysis (FIG. 2B). For serotype C, strains CcD43 and CcD130 were positive, with reactivities of 86%±5 and 108%±26 respectively. The Western blot analysis confirmed that they both had a serotype C capsule (FIG. 2C). For serotype D, 3 dog strains were strongly recognized and confirmed by Western blot: CcD16 (86%±14), CcD89 (95%±9) and CcD117 (99%±12) (FIG. 2D). Finally for serotype E, strain CcD96 displayed a high reactivity of 118%±37 and strains CcD20 and CcD106 displayed intermediate reactivities of respectively 57%±24 and 59%±24 while some strains presented a limited reactivity. All the strains with a value equal or higher than 30% were checked by Western blot and only one strain, only CcD96 was found to belong to serotype E (FIG. 2E). The complete results from the ELISA screenings and the Western blot analysis are summarized in FIG. 3. While all the strains isolated from patients belonged to serotype, A, B, C, D or E, 84.6% of the strains isolated from dogs were left untyped. In conclusion, the prevalence of serotype A was significantly increased by 22.9 fold in clinical isolates as compared to dog-hosted strains (Fisher's exact test, p=6.45×10⁻⁶). The prevalence of serotype B was increased by 14.6 fold, which was also statistically significant (Fisher's exact test, p=0.00123). Finally, a 4.2 fold increase was found for the serotype C, but it was not statistically significant (Fisher's exact test, p=0.00831). Finally, there is no significant difference in the prevalence of serotypes D and E (p values of 0.657 and 0.547 respectively in Fisher's exact test).

In conclusion, the Applicants found that, contrary to what was seen among human isolates, serotypes A, B and C were rarely found among the strains isolated from dog mouths. This indicates that all C. canimorsus strains are not equally dangerous for humans and that the strains belonging to serotypes A, B and C are more virulent for humans than strains from other serotypes.

### Example 4. Detection of the Capsular Serotypes A to E by PCR

The fact that only three capsular serotypes were more represented among clinical isolates than among dog isolates suggests that the presence of these capsules could be linked to a higher virulence. The Applicants thus tried to develop a PCR-based method using different oligonucleotides couples that would allow the identification of the 5 serotypes found among clinical isolates.

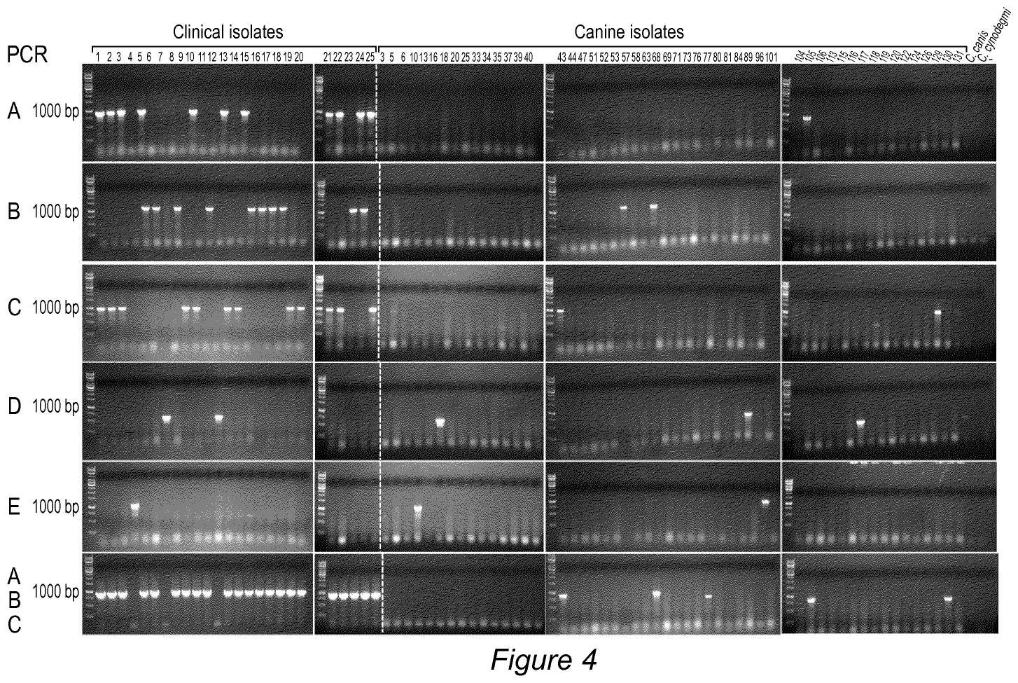

The Applicants thus first compared the loci encoding the capsule and LOS biosynthesis in the seven available genomes of C. canimorsus strains belonging to the five serotypes (Cc5, Cc2, Cc6, Cc11, Cc9, Cc12 and Cc4). Looking for a gene that was specific to serotype A strains (Cc5, Cc2), the Applicants identified an A4galT-like glycosyltransferase gene (Ccan_23210 (SEQ ID NO: 1) and CCAN2_1920004 (SEQ ID NO: 1) in Cc5 and Cc2 respectively) (Renzi F. et al., Scientific Reports, 2016). Two amplimers (called SeroA-fw and SeroA-rev) were designed and our complete C. canimorsus collection was tested by PCR. As shown in FIG. 4 this analysis detected only the 11 serotype A clinical isolates and the unique dog isolate (CcD105) of this serotype.

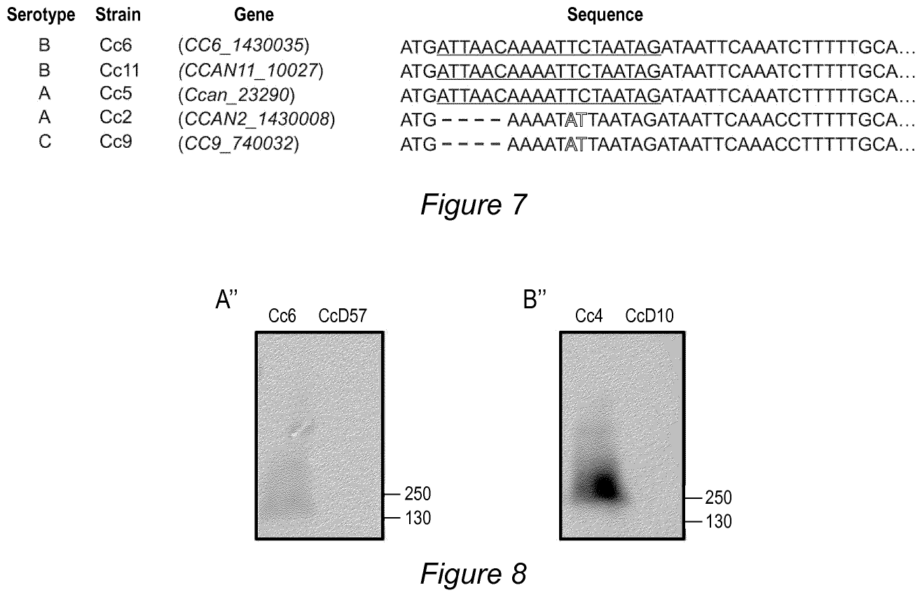

Regarding the B serotype, the Applicants could not identify any gene that was unique to the Cc6 and Cc11 genomes. All the genes of the CPS/LOS biosynthesis and transport loci of Cc6 and Cc11 had homologs in Cc2 and Cc5. However, aligning genes CC6_1430035 (SEQ ID NO: 2 and SEQ ID NO: 33) and CCAN11_10027 (SEQ ID NO: 49 and SEQ ID NO:33), encoding a putative family 1 glycosyltransferase, with their homologs from Cc2 (CCAN2_1430008, SEQ ID NO:34) and Cc9 (CC9_740032, SEQ ID NO: 34) (Renzi F. et al, Scientific Reports, 2016) revealed a difference in the 16 base pairs immediately downstream of the start codon (FIG. 7). Since both serotype B strains (Cc6 and Cc11) had the exact same gene sequence, shared by only one of the two A serotype strains (Cc5), the Applicants tested whether the exact same gene sequence would not be shared by all serotype B strains and the Applicants thus designed two oligonucleotides to amplify this specific gene region. As shown in FIG. 4, by this PCR, the Applicants could indeed detect all the 7 clinical strains as well as the only dog strain (CcD68) belonging to the B serotype. As expected the Applicants could also detect Cc5 but two other serotype A strains, namely Cc15 and Cc24, as well. Surprisingly, the PCR gave a positive result for one dog isolate (CcD57) that did not belong to any of the 5 serotypes (Table 5, FIG. 2E and FIG. 8A) and thus represents a false positive. Nevertheless, by this PCR the Applicants could detect all B serotype strains of our collection and this analysis, if combined with the one specific for the A serotype, allows the discrimination between serotypes A and B.

Regarding serotype C, the Applicants could not identify any gene unique to the Cc9 genome. Nevertheless, the Applicants identified one gene, CC9_740031 (SEQ ID NO: 3), encoding a putative O antigen polymerase (wzy) that has an homolog only in one seroype A strain, namely in Cc2. The Applicants thus tested whether this gene would be shared by all C serotype strains. As shown in FIG. 4, the Applicants could detect all serotype C strains namely the 4 clinical (Cc9, Cc14, Cc19 and Cc20) as well as the two dog isolates CcD43 and CcD130. This PCR thus allows the detection of the C serotype strains and, if combined to the one for the A serotype, to discriminate between these two serotypes.

Concerning serotype D, the Cc12 CPS/LOS locus was previously shown to be very divergent from the ones of A, B and C serotypes strains with a limited number of conserved genes (Renzi F. et al, Scientific Reports, 2016). The Applicants chose to amplify gene CCAN12_760043 (SEQ ID NO:4) encoding a putative lipopolysaccharide biosynthesis O-acetyl transferase (wbbJ) that had no homologs in all the other serotypes loci. As shown in FIG. 4, the Applicants could exclusively detect the serotype D strains and the Applicants detected them all (Cc12, Cc7, CcD16, CcD89 and CcD117).

Finally, as for Cc12, the serotype E strain Cc4 LPS/capsule locus strongly diverged from the ones of all the other serotypes. The Applicants thus chose as target gene a Cc4 unique gene, namely CC4_530066 (SEQ ID NO:5), encoding a glycosyltransferase 1 family protein. As shown in FIG. 4, this PCR detected the Cc4 and CcD96 serotype E strains. Among the other isolates, only the CcD10 strain gave a positive result although it did not react with the C antiserum (Table 5 and FIG. 8B) and could thus be considered as a false positive. In conclusion, capsular serotyping can be done by PCR with a very limited margin of error (2 false positive dog strains).

Next, given the higher prevalence of serotypes A, B and C (22/25) among clinical isolates, the Applicants decided to develop a PCR that would allow to detect all serotype A, B and C strains. To this aim, taking advantage of the high similarity among the CPS/LOS loci of the A B C serotypes belonging strains, the Applicants designed two amplimers specific to the conserved region of the putative glycosyltransferase wfdR orthologs genes of serotype A (Ccan_23240 in Cc5 (SEQ ID NO:6) and CCAN2_1430002 in Cc2 (SEQ ID NO:7)), serotype B (CC6_1430040 (SEQ ID NO:8) in Cc6 and CCAN11_2010013 (SEQ ID NO:6) in Cc11) and serotype C (CC9_740027, SEQ ID NO:7). As shown in FIG. 4, by this PCR the Applicants could exclusively detect all strains belonging to serotypes A, B and C.

This PCR, allowing to identify fast and specifically all the C. canimorsus strains belonging to the serotypes most represented in human infections, could thus be an extremely valuable tool in terms of prevention.

The Applicants have shown that the vast majority of the human infections is due to a limited number of capsular serotypes that are more virulent. Furthermore, the Applicants provide the bases for the prevention of these dramatic infections. More particularly, the Applicants provide (i) the reliable detection (i.e. a very limited number of false positives and no false negatives) of potentially more dangerous dogs by, for instance, a PCR reaction carried out directly on the dog's saliva and monitoring simultaneously the three dangerous serotypes and/or (ii) the vaccination of individuals at risk that are in contact with a dog by a combination of the three capsular antigens A, B and C.

The LOS and CPS synthesis are genetically linked in strain Cc5, resulting in similar polysaccharide units compositions in both structures. For serotypes B to E, the Applicants also found shared epitopes between the CPS and LOS. Even more, the antiserum directed against the CPS/LOS from serotype C recognized the LOS but not the CPS from some but not all strains of serotype A (data not shown), revealing some complexity in the CPS/LOS relation. Because of this complexity and because it is the CPS rather than the LOS that impacts the host-pathogen interaction (Renzi F. et al, Scientific Reports, 2016), the Applicants based their typing scheme on the CPS only. However, because of this cross-reaction, the distinction between serotypes A and C must be done by western blotting and not by immuno-fluorescence or ELISA. Because western blotting is a tedious technique for clinical laboratories, the Applicants set up a PCR approach for the capsular serotyping. This cross-reaction between the LOS of serotype A and some strains of serotype C also appeared when the typing was done by PCR but combining the two PCR reactions allows to determine the serotype without any ambiguity. Further work will be required to understand the molecular mechanisms underlying these LOS cross-reactions but carbohydrate chemistry always represents a long term project.

TABLE 5
Capsular serotyping of C. canimorsus strains isolated from dog mouths by ELISA

| | % of reactivity (normalized with respect to the type strain) ± SD | | | | |
|---|---|---|---|---|---|
| | Serotype A | Serotype B | Serotype C | Serotype D | Serotype E |
| Cc5 | 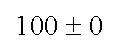 | 20 ± 8 | 27 ± 11 | 24 ± 6 | 17 ± 2 |
| Cc5 Y1C12 | 14 ± 6 | Nd | nd | nd | nd |
| Cc6 | 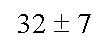 | 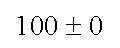 | 24 ± 12 | 20 ± 4 | 13 ± 2 |
| Cc6 Δ wbuB | nd | 14 ± 7 | nd | nd | nd |
| Cc9 | 15 ± 3 | 17 ± 7 | 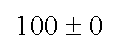 | 22 ± 4 | 17 ± 5 |
| Cc9 Δ wbuB | nd | Nd | 20 ± 7 | nd | nd |
| Cc12 | 19 ± 7 | 15 ± 5 | 23 ± 8 | 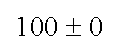 | 18 ± 0 |
| Cc12 Δ wbtA | nd | Nd | nd | 20 ± 5 | nd |
| Cc4 | 16 ± 3 | 14 ± 3 | 30 ± 10 | 26 ± 5 | 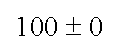 |
| CcD3 | 18 ± 7 | 11 ± 4 | 18 ± 5 | 19 ± 3 | 14 ± 6 |
| CcD5 | 17 ± 4 | 13 ± 8 | 16 ± 4 | 22 ± 4 | 13 ± 6 |
| CcD6 | 18 ± 10 | 11 ± 5 | 20 ± 7 | 18 ± 3 | 15 ± 7 |
| CcD10 | 17 ± 8 | 12 ± 5 | 18 ± 3 | 17 ± 2 | 17 ± 5 |
| CcD13 | 16 ± 7 | 11 ± 5 | 21 ± 4 | 17 ± 2 | 12 ± 5 |
| CcD16 | 18 ± 7 | 10 ± 4 | 17 ± 4 | 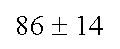 | 11 ± 5 |
| CcD18 | 19 ± 9 | 12 ± 6 | 17 ± 5 | 15 ± 2 | 28 ± 12 |
| CcD20 | 17 ± 6 | 11 ± 6 | 19 ± 3 | 17 ± 3 | 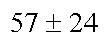 |
| CcD25 | 15 ± 6 | 11 ± 5 | 18 ± 4 | 17 ± 2 | 12 ± 5 |
| CcD33 | 20 ± 9 | 11 ± 6 | 17 ± 6 | 22 ± 2 | 13 ± 5 |
| CcD34 | 16 ± 9 | 10 ± 4 | 16 ± 3 | 14 ± 2 | 13 ± 6 |
| CcD35 | 14 ± 7 | 12 ± 4 | 15 ± 5 | 12 ± 1 | 12 ± 4 |
| CcD37 | 15 ± 4 | 9 ± 3 | 19 ± 2 | 16 ± 0 | 12 ± 4 |
| CcD39 | 14 ± 4 | 10 ± 5 | 18 ± 3 | 22 ± 2 | 14 ± 6 |
| CcD40 | 16 ± 8 | 11 ± 5 | 19 ± 4 | 19 ± 4 | 12 ± 5 |
| CcD43 | 20 ± 10 | 24 ± 14 | 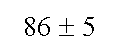 | 17 ± 1 | 13 ± 5 |
| CcD44 | 15 ± 8 | 9 ± 4 | 25 ± 7 | 16 ± 0 | 12 ± 5 |
| CcD47 | 16 ± 6 | 11 ± 6 | 17 ± 3 | 18 ± 0 | 12 ± 5 |
| CcD51 | 15 ± 7 | 14 ± 5 | 18 ± 4 | 20 ± 4 | 11 ± 5 |
| CcD52 | 16 ± 8 | 11 ± 7 | 16 ± 5 | 20 ± 6 | 11 ± 5 |
| CcD53 | 19 ± 8 | 12 ± 6 | 17 ± 2 | 18 ± 2 | 12 ± 5 |
| CcD57 | 17 ± 6 | 28 ± 18 | 21 ± 4 | 23 ± 12 | 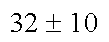 |
| | % of reactivity (normalized with type strain) ± SD | | | | |
| Strain | Serotype A | Serotype B | Serotype C | Serotype D | Serotype E |
| CcD58 | 18 ± 7 | 11 ± 5 | 17 ± 3 | 22 ± 3 | 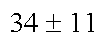 |
| CcD63 | 15 ± 9 | 11 ± 5 | 17 ± 1 | 17 ± 0 | 29 ± 11 |
| CcD68 | 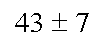 | 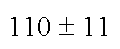 | 16 ± 5 | 18 ± 0 | 13 ± 5 |
| CcD69 | 14 ± 7 | 11 ± 6 | 16 ± 3 | 13 ± 2 | 12 ± 5 |
| CcD71 | 15 ± 6 | 11 ± 5 | 17 ± 6 | 16 ± 2 | 13 ± 6 |
| CcD73 | 19 ± 7 | 12 ± 7 | 17 ± 0 | 23 ± 3 | 15 ± 6 |
| CcD76 | 13 ± 6 | 16 ± 8 | 14 ± 1 | 15 ± 3 | 15 ± 7 |
| CcD77 | 14 ± 9 | 13 ± 4 | 17 ± 7 | 14 ± 2 | 15 ± 7 |
| CcD80 | 16 ± 11 | 13 ± 4 | 19 ± 5 | 17 ± 3 | 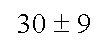 |
| CcD81 | 16 ± 5 | 12 ± 4 | 23 ± 7 | 19 ± 4 | 22 ± 5 |
| CcD84 | 17 ± 8 | 14 ± 7 | 13 ± 1 | 18 ± 2 | 29 ± 16 |
| CcD89 | 17 ± 6 | 15 ± 1 | 20 ± 6 | 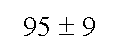 | 14 ± 9 |
| CcD96 | 20 ± 8 | 14 ± 1 | 19 ± 7 | 15 ± 1 | 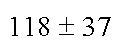 |
| CcD101 | 18 ± 5 | 12 ± 3 | 13 ± 4 | 16 ± 1 | 13 ± 7 |
| CcD104 | 16 ± 9 | 14 ± 1 | 18 ± 7 | 21 ± 4 | 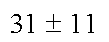 |
| CcD105 | 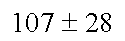 | 14 ± 2 | 16 ± 8 | 17 ± 5 | 14 ± 8 |
| CcD106 | 13 ± 8 | 16 ± 1 | 21 ± 11 | 17 ± 2 | 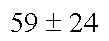 |
| CcD113 | 15 ± 11 | 16 ± 2 | 19 ± 9 | 14 ± 0 | 14 ± 6 |
| CcD115 | 15 ± 9 | 16 ± 2 | 19 ± 9 | 21 ± 4 | 16 ± 8 |
| CcD116 | 14 ± 8 | 14 ± 2 | 19 ± 5 | 18 ± 2 | 15 ± 7 |
| CcD117 | 19 ± 10 | 16 ± 1 | 19 ± 8 | 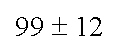 | 14 ± 7 |
| CcD118 | 15 ± 7 | 14 ± 1 | 20 ± 9 | 19 ± 4 | 15 ± 7 |
| CcD119 | 15 ± 8 | 16 ± 2 | 16 ± 9 | 28 ± 22 | 15 ± 9 |
| CcD120 | 15 ± 8 | 17 ± 3 | 17 ± 5 | 23 ± 7 | 16 ± 8 |
| CcD122 | 12 ± 8 | 14 ± 1 | 17 ± 6 | 13 ± 1 | 14 ± 8 |
| CcD124 | 15 ± 8 | 16 ± 1 | 19 ± 8 | 19 ± 4 | 15 ± 7 |
| CcD126 | 13 ± 7 | 14 ± 1 | 16 ± 6 | 21 ± 3 | 15 ± 7 |
| CcD129 | 13 ± 9 | 15 ± 2 | 21 ± 6 | 20 ± 2 | 16 ± 8 |
| CcD130 | 13 ± 7 | 29 ± 11 | 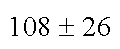 | 19 ± 2 | 11 ± 6 |
| CcD131 | 12 ± 7 | 14 ± 2 | 17 ± 6 | 19 ± 3 | 14 ± 6 |

Capsular serotyping was determined by ELISA on entire heat-killed bacteria. The following sera were used: Y1C12 adsorbed anti-Cc5 (A), Cc6 ΔwbuB adsorbed anti-Cc6 (B), Cc9 ΔwbuB adsorbed anti-Cc9 (C), Cc12 ΔwbtA adsorbed anti-Cc12 (D) and anti-Cc4 adsorbed with all clinical strains except Cc4 (E). Reactivities of the strains with the sera tested were calculated with respect to the value of the corresponding type strain.
Values are the mean (± standard deviation, SD) of at least 3 independant experiments. The type strains for each capsular serotype and the dog strains with strong reactivities (>80%) are highlighted in dark grey. The dog strains presenting intermediate reactivities comprised between 30 and 60% are highlighted in light grey.
nd: not determined; SD: standard deviation

TABLE 6
Synteny statistics

| Cc5 (serotype A) | | | Cc6 (serotype B) | | | Cc9 (serotype C) | | | Cc12 (serotype D) | | | Cc4 (serotype E) | | |
|---|---|---|---|---|---|---|---|---|---|---|---|---|---|---|
| CDS in syntons | | | CDS in syntons | | | CDS in syntons | | | CDS in syntons | | | CDS in syntons | | |
| Number | % | Strain | Number | % | Strain | Number | % | Strain | Number | % | Strain | Number | % | Strain |
| 2220 | 88.13 | Cc2 (A) | 2240 | 93.80 | Cc5 (A) | 2053 | 84.00 | Cc5 (A) | 2322 | 85.94 | Cc5 (A) | 1938 | 85.56 | Cc5 (A) |
| 2245 | 89.12 | Cc6 (B) | 2181 | 91.33 | Cc2 (A) | 2015 | 82.45 | Cc2 (A) | 2263 | 83.75 | Cc2 (A) | 1895 | 83.66 | Cc2 (A) |
| 2020 | 80.19 | Cc11 | 1965 | 82.29 | Cc11 | 2024 | 82.82 | Cc6 (B) | 2284 | 84.53 | Cc6 (B) | 1926 | 85.03 | Cc6 (B) |
| | | (B) | | | (B) | | | | | | | | | |
| 2076 | 82.41 | Cc9 (C) | 2031 | 85.05 | Cc9 (C) | 1976 | 80.85 | Cc11 | 2220 | 82.16 | Cc11 | 1856 | 81.94 | Cc11 |
| | | | | | | | | (B) | | | (B) | | | (B) |
| 1947 | 77.29 | Cc12 | 1911 | 80.03 | Cc12 | 1885 | 77.13 | Cc12 | 2307 | 85.38 | Cc9 (C) | 1943 | 85.78 | Cc9 (C) |
| | | (D) | | | (D) | | | (D) | | | | | | |
| 1980 | 78.60 | Cc4 (E) | 1949 | 81.62 | Cc4 (E) | 1966 | 80.44 | Cc4 (E) | 2360 | 87.34 | Cc4 (E) | 1933 | 85.34 | Cc12 |
| | | | | | | | | | | | | | | (D) |

TABLE 7
Strain information

| | | Genome/ | |
|---|---|---|---|
| Strain | | LOS-CPS locus | |
| ID | Collection | accession number | Reference |
| Cc1 | BCCM/LMG 11511; CCUG | . . . | [1] |
| | 17234; strain P810; strain SSI | | |
| | P810 | | |
| Cc2 | CCUG 70775 | CDOJ00000000 | [2] |
| Cc3 | . . . | . . . | [3] |
| Cc4 | CCUG 70776 | PRJEB20110 | [4] |
| Cc5 | BCCM/LMG 28512; CCUG | NC_015846.1 | [5] |
| | 70777 | | |
| Cc6 | CCUG 70778 | PRJEB20107 | [6] |
| Cc7 | . . . | . . . | [5] |
| Cc8 | . . . | . . . | [6] |
| Cc9 | BCCM/LMG 11510, CCUG | PRJEB20108 | [7] |
| | 12569, CDC A3626 | | |
| Cc10 | BCCM/G 11541, CCUG 24741, | . . . | [7] |
| | ATCC 35978, CDC 08936 | | |
| Cc11 | BCCM/LMG 11551, MCCM | CDOK00000000 | [7] |
| | 01373 | | |
| Cc12 | ATCC 35979, CDC 7120, | CDOE00000000 | [8] |
| | CCUG 53895 | | |
| Cc13 | . . . | . . . | [9] |
| Cc14 | . . . | . . . | [6] |
| Cc15 | . . . | . . . | [6] |
| Cc16 | . . . | . . . | [6] |
| Cc17 | . . . | . . . | [6] |
| Cc18 | . . . | . . . | [6] |
| Cc19 | . . . | . . . | [6] |
| Cc20 | CCUG 55909 | . . . | [10] |
| Cc21 | CCUG 60839 | . . . | [10] |
| Cc22 | CCUG 20318 | . . . | [10] |
| Cc23 | CCUG 48899 | . . . | [10] |
| Cc24 | CCUG 67384 | . . . | [10] |
| Cc25 | CCUG 66222 | . . . | [10] |

### REFERENCES

1. Heltberg, O., et al., The cultivation and rapid enzyme identification of DF-2. Eur J Clin Microbiol, 1984. 3(3): p. 241-3.
2. Hantson, P., et al., Fatal Capnocytophaga canimorsus septicemia in a previously healthy woman. Ann Emerg Med, 1991. 20(1): p. 93-4.
3. Vanhonsebrouck, A. Y., et al., Fatal septicemia with Capnocytophaga canimorsus in a compromised host. A case report with review of the literature. Acta Clin Belg, 1991. 46(6): p. 364-70.
4. Rougemont, M., et al., Capnocytophaga canimorsus prosthetic aortitis in an HIV-positive woman. J Clin Microbiol, 2013. 51(8): p. 2769-71.
5. Shin, H., et al., Escape from immune surveillance by Capnocytophaga canimorsus. J Infect Dis, 2007. 195(3): p. 375-86.
6. Renzi, F., et al., Only a subset of C. canimorsus strains is dangerous for humans. Emerg Microbes Infect, 2016. 5: p. e29.
7. Vandamme, P., et al., Polyphasic analysis of strains of the genus Capnocytophaga and Centers for Disease Control group DF-3. Int J Syst Bacteriol, 1996. 46(3): p. 782-91.
8. Butler, T., et al., Unidentified gram-negative rod infection. A new disease of man. Ann Intern Med, 1977. 86(1): p. 1-5.
9. Kleijnen-Grebien, B., et al., Fatal case of sepsis with Capnocytophaga canimorsus after a minor dog bite. Ned Tijdschr Geneeskd, 2008. 152(34): p. 1882-5.
10. Hess, E., et al., Identification of Virulent Capnocytophaga canimorsus Isolates by Capsular Typing. J Clin Microbiol, 2017. 55(6): p. 1902-1914.

1. A method for identifying C. canimorsus in a sample as one of serotype A, serotype B, serotype C, serotype D or serotype E, said identifying is based on one or more of the following steps:
contacting said sample or at least a portion of nucleic acids isolated from said sample under conditions conducive to polymerase-based nucleic acid amplification with an amplification primer pair configured to amplify a target nucleic acid region in a gene A of the CPS/LOS biosynthesis and transport loci of C. canimorsus wherein said gene A has syntenic orthologs in C. canimorsus strains Cc1, Cc2, Cc3, Cc5, Cc10, Cc13, Cc15, Cc21, Cc22, Cc24 and Cc25 and not in C. canimorsus strains Cc4, Cc6, Cc7, Cc8, Cc9, Cc11, Cc12, Cc14, Cc16, Cc17, Cc18, Cc19, Cc20 and Cc23; an amplification primer pair configured to amplify a target nucleic acid region in a gene B of the LOS/capsule biosynthesis and transport loci of C. canimorsus wherein said gene B has syntenic orthologs in C. canimorsus strains Cc6, Cc8, Cc11, Cc16, Cc17, Cc18 and Cc23 and not C. canimorsus strains Cc1, Cc2, Cc3, Cc4, Cc5, Cc7, Cc9, Cc10, Cc12, Cc13, Cc14, Cc15, Cc19, Cc20, Cc21, Cc22, Cc24 and Cc25; an primer pair configured to amplify a target nucleic acid region in a gene C of the LOS/capsule biosynthesis and transport loci of C. canimorsus wherein said gene C has syntenic orthologs in C. canimorsus strains Cc9, Cc14, Cc19 and Cc20 and not in C. canimorsus strains Cc1, Cc2, Cc3, Cc4, Cc5, Cc6, Cc7, Cc8, Cc10, Cc11, Cc12, Cc13, Cc15, Cc16, Cc17, Cc18, Cc21, Cc22, Cc23, Cc24 and Cc25; an amplification primer pair configured to amplify a target nucleic acid region in a gene D of the CPS/LOS biosynthesis and transport loci of C. canimorsus wherein said gene D has syntenic orthologs in C. canimorsus strains Cc7 and Cc12 and not in C. canimorsus strains Cc1, Cc2, Cc3, Cc4, Cc5, Cc6, Cc8, Cc9, Cc10, Cc11, Cc13, Cc14, Cc15, Cc16, Cc17, Cc18, Cc19, Cc20, Cc21, Cc22, Cc23, Cc24 and Cc25; an amplification primer pair configured to amplify a target nucleic acid region in a gene E of the CPS/LOS biosynthesis and transport loci of C. canimorsus, wherein said gene E is unique to C. canimorsus strain Cc4; and/or an amplification primer pair configured to amplify a target nucleic acid region in a gene ABC of the CPS/LOS biosynthesis and transport loci of C. canimorsus wherein said gene ABC has syntenic orthologs in C. canimorsus strains Cc1, Cc2, Cc3, Cc5, Cc6, Cc8, Cc9, Cc10, Cc11, Cc13, Cc14, Cc15, Cc16, Cc17, Cc18, Cc19, Cc20, Cc21, Cc22, Cc23, Cc24 and Cc25 and not in C. canimorsus strains Cc4, Cc7 and Cc12; and one or more of the following steps:
detecting the amplified target nucleic acid region in said gene A, and optionally simultaneously not detecting the amplified target nucleic acid region in said gene B, C, D and E, whereby C. canimorsus in the sample is identified as capsular serotype A;
detecting the amplified target nucleic acid region in said gene B, and simultaneously not detecting the amplified target nucleic acid region in said gene A, C, D and E, whereby C. canimorsus in the sample is identified as capsular serotype B;
detecting the amplified target nucleic acid region in said gene C, and simultaneously not detecting the amplified target nucleic acid region in said gene A, B, D and E, whereby C. canimorsus in the sample is identified as capsular serotype C;
detecting the amplified target nucleic acid region in said gene D, and simultaneously not detecting the amplified target nucleic acid region in said gene A, B, C, E and ABC, whereby C. canimorsus in the sample is identified as capsular serotype D in the sample is detected;
detecting the amplified target nucleic acid region in said gene E, and simultaneously not detecting the amplified target nucleic acid region in said gene A, B, C, D and ABC, whereby C. canimorsus in the sample is identified as capsular serotype E; and/or
detecting the amplified target nucleic acid region in said gene ABC, and simultaneously not detecting the amplified target nucleic acid region in said gene D and E, whereby C. canimorsus in the sample is identified as capsular serotype A, B and/or C; or alternatively
by contacting said sample with one or more polyclonal antibodies specifically recognizing capsular polysaccharides (CPS) and/or lipooligosaccharides (LOS) of wild-type C. canimorsus bacteria of one or more but not all capsular serotypes which is obtained by adsorbing anti-serum obtained by immunization of a non-human animal with a composition comprising wild-type C. canimorsus bacteria of said one or more capsular serotypes subsequently or simultaneously with one or more wild type C. canimorsus strains selected from a wild-type C. canimorsus strain of the capsular serotype A, a wild-type C. canimorsus strain of the capsular serotype B, a wild-type C. canimorsus strain of the capsular serotype C, a wild-type C. canimorsus strain of the capsular serotype D and a wild-type C. canimorsus strain of the capsular serotype E, but not with wild-type C. canimorsus strains of the one or more capsular serotypes used for immunization.

2. The method for identifying C. canimorsus in a sample as one of serotype A, serotype B, serotype C, serotype D or serotype E of claim 1,
wherein said gene A is A4GalT-like glycosyltransferase gene (A4galT GT), preferably wherein A4galT GT has a coding nucleic acid sequence that is at least 90%, preferably at least 95%, more preferably 100% identical to SEQ ID NO: 1;
wherein said gene B is a first family 1 glycosyltransferase gene (GT1), preferably wherein said first GT1 has a coding nucleic acid sequence that is at least 90%, preferably at least 95%, more preferably 100% identical to SEQ ID NO: 2 and/or 49;
wherein said gene C is wzy, preferably wherein wzy has a coding nucleic acid sequence that is at least 90%, preferably at least 95%, more preferably 100% identical to SEQ ID NO: 3;
wherein said gene D is wbbJ, preferably wherein wbbJ has a coding nucleic acid that is at least 90%, preferably at least 95%, more preferably 100% identical to SEQ ID NO: 4,
wherein said gene E is a second GT1, preferably wherein said second GT1 has a coding nucleic acid sequence that is at least 90%, preferably at least 95%, identical to SEQ ID NO: 5; and
wherein said gene ABC is wfdR, preferably wherein wfdR has a coding nucleic acid sequence that is at least 90%, preferably at least 95%, more preferably 100% identical to SEQ ID NO: 6, 7 and/or 8.

3. The method according to claim 2, wherein:
the amplification primer pair configured to amplify a target nucleic acid region in A4galT GT comprises a first amplification primer which comprises a nucleotide sequence which is at least 90%, preferably at least 95%, identical to SEQ ID NO: 9 or SEQ ID NO: 11 and a second amplification primer which comprises a nucleotide sequence which is at least 90%, preferably at least 95%, identical to SEQ ID NO: 10 or SEQ ID NO: 12;
the amplification primer pair configured to amplify a target nucleic acid region in the first GT1 comprises a first amplification primer which comprises a nucleotide sequence which is at least 90%, preferably at least 95%, identical to SEQ ID NO: 13 or SEQ ID NO: 15 and a second amplification primer which comprises a nucleotide sequence which is at least 90%, preferably at least 95%, identical to SEQ ID NO: 14 or SEQ ID NO: 16;
the amplification primer pair configured to amplify a target nucleic acid region in wzy comprises a first amplification primer which comprises a nucleotide sequence which is at least 90%, preferably at least 95%, identical to SEQ ID NO: 17 and a second amplification primer which comprises a nucleotide sequence which is at least 90%, preferably at least 95%, identical to SEQ ID NO: 18;
the amplification primer pair configured to amplify a target nucleic acid region in wbbJ comprises a first amplification primer which comprises a nucleotide sequence which is at least 90%, preferably at least 95%, identical to SEQ ID NO: 19 and a second amplification primer which comprises a nucleotide sequence which is at least 90%, preferably at least 95%, identical to SEQ ID NO: 20;
the amplification primer pair configured to amplify a target nucleic acid region in the second GT1 comprises a first amplification primer which comprises a nucleotide sequence which is at least 90%, preferably at least 95%, identical to SEQ ID NO: 21 and a second amplification primer which comprises a nucleotide sequence which is at least 90%, preferably at least 95%, identical to SEQ ID NO: 22; and/or
the amplification primer pair configured to amplify a target nucleic acid region in wfdR comprises a first amplification primer which comprises a nucleotide sequence which is at least 90%, preferably at least 95%, identical to SEQ ID NO: 23 and a second amplification primer which comprises a nucleotide sequence which is at least 90%, preferably at least 95%, identical to SEQ ID NO: 24.

4. The method according to claim 1, wherein the polymerase-based nucleic acid amplification is multiplexed, such that at least two of the target nucleic acid regions are amplified in the same polymerase-based nucleic acid amplification reaction.

5. The method according to claim 1, wherein the polymerase-based nucleic acid amplification is polymerase chain reaction (PCR).

6. A set of amplification primer pairs suitable for polymerase-based nucleic acid amplification, comprising an amplification primer pair configured to amplify a target nucleic acid region in a gene A of the CPS/LOS biosynthesis and transport loci of C. canimorsus wherein said gene A has syntenic orthologs in C. canimorsus strains Cc1, Cc2, Cc3, Cc5, Cc10, Cc13, Cc15, Cc21, Cc22, Cc24 and Cc25 and not in C. canimorsus strains Cc4, Cc6, Cc7, Cc8, Cc9, Cc11, Cc12, Cc14, Cc16, Cc17, Cc18, Cc19, Cc20 and Cc23; an amplification primer pair configured to amplify a target nucleic acid region in a gene B of the LOS/capsule biosynthesis and transport loci of C. canimorsus wherein said gene B has syntenic orthologs in C. canimorsus strains Cc6, Cc8, Cc11, Cc16, Cc17, Cc18 and Cc23 and not C. canimorsus strains Cc1, Cc2, Cc3, Cc4, Cc5, Cc7, Cc9, Cc10, Cc12, Cc13, Cc14, Cc15, Cc19, Cc20, Cc21, Cc22, Cc24 and Cc25; an amplification primer pair configured to amplify a target nucleic acid region in a gene C of the LOS/capsule biosynthesis and transport loci of C. canimorsus wherein said gene C has syntenic orthologs in C. canimorsus strains Cc9, Cc14, Cc19 and Cc20 and not in C. canimorsus strains Cc1, Cc2, Cc3, Cc4, Cc5, Cc6, Cc7, Cc8, Cc10, Cc11, Cc12, Cc13, Cc15, Cc16, Cc17, Cc18, Cc21, Cc22, Cc23, Cc24 and Cc25; an amplification primer pair configured to amplify a target nucleic acid region in a gene D of the CPS/LOS biosynthesis and transport loci of C. canimorsus wherein said gene D has syntenic orthologs in C. canimorsus strains Cc7 and Cc12 and not in C. canimorsus strains Cc1, Cc2, Cc3, Cc4, Cc5, Cc6, Cc8, Cc9, Cc10, Cc11, Cc13, Cc14, Cc15, Cc16, Cc17, Cc18, Cc19, Cc20, Cc21, Cc22, Cc23, Cc24 and Cc25; an amplification primer pair configured to amplify a target nucleic acid region in a gene E of the CPS/LOS biosynthesis and transport loci of C. canimorsus, wherein said gene E is unique to C. canimorsus strain Cc4; and/or an amplification primer pair configured to amplify a target nucleic acid region in a gene ABC of the CPS/LOS biosynthesis and transport loci of C. canimorsus wherein said gene ABC has syntenic orthologs in C. canimorsus strains Cc1, Cc2, Cc3, Cc5, Cc6, Cc8, Cc9, Cc10, Cc11, Cc13, Cc14, Cc15, Cc16, Cc17, Cc18, Cc19, Cc20, Cc21, Cc22, Cc23, Cc24 and Cc25 and not in C. canimorsus strains Cc4, Cc7 and Cc12.

7. The set of amplification primer pairs according to claim 6, wherein said gene A is C. canimorsus A4galT GT, wherein the coding nucleic acid sequence of said gene is at least 90%, preferably at least 95%, more preferably 100% identical to SEQ ID NO: 1; said gene B is C. canimorsus GT1, wherein the coding nucleic acid sequence of said gene is at least 90%, preferably at least 95%, more preferably 100% identical to SEQ ID NO: 2 and/or 49; said gene C is C. canimorsus wzy, wherein the coding nucleic acid sequence of said gene is at least 90%, preferably at least 95%, more preferably 100% identical to SEQ ID NO: 3; said gene D is C. canimorsus wbbJ; wherein the coding nucleic acid sequence of said gene is at least 90%, preferably at least 95%, more preferably 100% identical to SEQ ID NO: 4; said gene E is a second C. canimorsus GT1, wherein the coding nucleic acid sequence of said gene is at least 90%, preferably at least 95%, identical to SEQ ID NO: 5 and/or said gene ABC is C. canimorsus wfdR, wherein the coding nucleic acid sequence of said gene is at least 90%, preferably at least 95%, more preferably 100% identical to SEQ ID NO: 6, SEQ ID NO:7 and/or SEQ ID NO:8.

8. The set of amplification primer pairs according to claim 7, wherein:
the amplification primer pair configured to amplify a target nucleic acid region in A4galT GT comprises a first amplification primer which comprises a nucleotide sequence which is at least 90%, preferably at least 95%, identical to SEQ ID NO: 9 or 11 and a second amplification primer which comprises a nucleotide sequence which is at least 90%, preferably at least 95%, identical to SEQ ID NO: 10 or 12;
the amplification primer pair configured to amplify a target nucleic acid region in the first GT1 comprises a first amplification primer which comprises a nucleotide sequence which is at least 90%, preferably at least 95%, identical to SEQ ID NO: 13 or 15 and a second amplification primer which comprises a nucleotide sequence which is at least 90%, preferably at least 95%, identical to SEQ ID NO: 14 or 16;
the amplification primer pair configured to amplify a target nucleic acid region in wzy comprises a first amplification primer which comprises a nucleotide sequence which is at least 90%, preferably at least 95%, identical to SEQ ID NO: 17 and a second amplification primer which comprises a nucleotide sequence which is at least 90%, preferably at least 95%, identical to SEQ ID NO: 18;
the amplification primer pair configured to amplify a target nucleic acid region in wbbJ comprises a first amplification primer which comprises a nucleotide sequence which is at least 90%, preferably at least 95%, identical to SEQ ID NO: 19 and a second amplification primer which comprises a nucleotide sequence which is at least 90%, preferably at least 95%, identical to SEQ ID NO: 20;
the amplification primer pair configured to amplify a target nucleic acid region in the second GT1 comprises a first amplification primer which comprises a nucleotide sequence which is at least 90%, preferably at least 95%, identical to SEQ ID NO: 21 and a second amplification primer which comprises a nucleotide sequence which is at least 90%, preferably at least 95%, identical to SEQ ID NO: 22; and/or
the amplification primer pair configured to amplify a target nucleic acid region in wfdR comprises a first amplification primer which comprises a nucleotide sequence which is at least 90%, preferably at least 95%, identical to SEQ ID NO: 23 and a second amplification primer which comprises a nucleotide sequence which is at least 90%, preferably at least 95%, identical to SEQ ID NO: 24.

9. The set of amplification primer pairs according to claim 6, wherein the amplification primer pairs are configured to allow for multiplexed polymerase-based nucleic acid amplification, such that at least two of the target nucleic acid regions can be amplified in the same polymerase-based nucleic acid amplification reaction.

10. The set of amplification primer pairs according to claim 6, provided as a kit of parts further comprising reagents sufficient for formulating a polymerase-based nucleic acid amplification reaction mixture.

11. A polyclonal antibody recognizing capsular polysaccharides (CPS) and/or lipooligosaccharides (LOS) of wild-type C. canimorsus bacteria of one or more but not all capsular serotypes which is obtained by adsorbing anti-serum obtained by immunization of a non-human animal with a composition comprising wild-type C. canimorsus bacteria of said one or more capsular serotypes subsequently or simultaneously with one or more wild type C. canimorsus strains selected from a wild-type C. canimorsus strain of the capsular serotype A, a wild-type C. canimorsus strain of the capsular serotype B, a wild-type C. canimorsus strain of the capsular serotype C, a wild-type C. canimorsus strain of the capsular serotype D and a wild-type C. canimorsus strain of the capsular serotype E, but not with wild-type C. canimorsus strains of the one or more capsular serotypes used for immunization.

12. (canceled)

13. The method of claim 1, which comprises a step of immuno-purifying said sample using one or more of said polyclonal antibodies; and a step of performing a C. canimorsus-specific PCR on said immuno-purified sample, preferably a C. canimorsus-specific 16S ribosomal DNA PCR.

14. The method according to claim 1, comprising subjecting said sample to an immuno-purification step prior to contacting said sample or at least a portion of nucleic acids isolated from said sample under conditions conducive to polymerase-based nucleic acid amplification with said amplification primer pair; wherein said immuno-purification step is performed using one or more of said polyclonal antibodies specifically recognizing capsular polysaccharides (CPS) and/or lipooligosaccharides (LOS) of wild-type C. canimorsus bacteria of one or more but not all capsular serotypes.

15. A polyvalent vaccine against C. canimorsus comprising
inactivated or attenuated cells of a C. canimorsus strain of the serotype A selected from the list consisting of C. canimorsus strains Cc1, Cc2, Cc3, Cc5, Cc10, Cc13, Cc15, Cc21, Cc22, Cc24 and Cc25, or fragments thereof;
inactivated or attenuated cells of a C. canimorsus strain of the serotype B selected from the list consisting of C. canimorsus strains Cc6, Cc8, Cc11, Cc16, Cc17, Cc18 and Cc23, or fragments thereof;
inactivated or attenuated cells of a C. canimorsus strain of the serotype C selected from the list consisting of C. canimorsus strains Cc9, Cc14, Cc19 and Cc20, or fragments thereof; and optionally
inactivated or attenuated cells of a C. canimorsus strain of the serotype D selected from the list consisting of C. canimorsus strains Cc12 and Cc7, or fragments thereof; and/or
inactivated or attenuated cells of a C. canimorsus strain of the serotype E, wherein said C. canimorsus strain is Cc4, or fragments thereof.

16-17. (canceled)

18. A method for the prevention of a bacterial infection with a pathogenic C. canimorsus in a subject, said method comprising administering, to said subject, a polyvalent vaccine against C. canimorsus comprising:
inactivated or attenuated cells of a C. canimorsus strain of the serotype A selected from the list consisting of C. canimorsus strains Cc1, Cc2, Cc3, Cc5, Cc10, Cc13, Cc15, Cc21, Cc22, Cc24 and Cc25, or fragments thereof;
inactivated or attenuated cells of a C. canimorsus strain of the serotype B selected from the list consisting of C. canimorsus strains Cc6, Cc8, Cc11, Cc16, Cc17, Cc18 and Cc23, or fragments thereof;
inactivated or attenuated cells of a C. canimorsus strain of the serotype C selected from the list consisting of C. canimorsus strains Cc9, Cc14, Cc19 and Cc20, or fragments thereof; and optionally
inactivated or attenuated cells of a C. canimorsus strain of the serotype D selected from the list consisting of C. canimorsus strains Cc12 and Cc7, or fragments thereof, and/or
inactivated or attenuated cells of a C. canimorsus strain of the serotype E, wherein said C. canimorsus strain is Cc4, or fragments thereof.

19. (canceled)


SEQUENCE LISTING (50 sequences)

SEQ ID NO: 1 - DNA, 903 bp, Capnocytophaga canimorsus
atgaaaaaagtaccaatagtttttatatttaaccaaggatatgctttaccagcgagtgtatgtattttttcattattacataataagaaagaggaagtaaattatcatatatgtattatgtatttaaggggacgactttcaagtcagactatttctaacataacagaaatggtttgtagttatggtagttgtagtatagagttcaaagaaatagaaaaccattttacaaataatagaactgtacgttatataactatcgatgcctactgtaagtttgaaataccaaaagtttttgaagattatgaaagagttatttacgcagatgtagatatgatttttgatggagatatatcagaattgatggatatatcttttaaggaatctattgcggcagtaagaattcctgtaggacacgtttccattgattttaaatcactcaatataaatgaagaaaattatttcaattctggattgatagtaatgaatacaaggaaaataaatgagaataagattattgaaaaaaaaatacgacctttgataggaaataaatatgaccacgtcgaccaagatatttttaatattgcatttaagggagatacttatttcttatcctctatatataacttctcttctgatagactcattaaagatagtgaagaacgtaacaagtctataaaggtatatcattacactggtgcaaagccttggaataaaggcgtatttccaatattttcagatatatggtgggaatattatagaagatctcctttctaccaagagaatgtttatttctcaaaacaatcagaatattatcagttacacagatatataaaattaggaactaagatagaaaaaatgggtgttttttctttgtggaatatattaaaaaagataaaaaaatga

SEQ ID NO: 2 - DNA, 1125 bp, Capnocytophaga canimorsus
atgattaacaaaattctaatagataattcaaatctttttgcaggtggaggtatacaagtggcaacatcttttttgaatgatttgaaagatatggatacttctccttacgtttttcatgtaatacaatcgtataatagcagtaaggctattgataaaacaaaatttcctaaagaattcatattttatgatttggaagaatctttgtcaaaaataaaaaaaatacaatacgtaaaagaaatagagaacaaagtcaaacccaattgtattttcactaccttcgggccatcttatcataagagtaattttccgaagattgttggttttgctttcggtcaaatgatttataaaaattcacccttttacaagaagctttcttttttgcaaaggctaaaatatgaatttattatttttatgaaaaaggagatgttccttaaaaattcagatgtacttatttttgaaacagatgacgctaaatttaaatttaatagattaacacataataagaaaatatcatatacggtaaataatacactgaatactatttttaccactccgaacgattggaaagattttaaaataaataaagtagggctgaatattctttgccttacggctaattatttacataaaaatctagatataattcctcgtgtaatagaccaacttcttacaatggataatgatttagattttaaattttttatttctttagaaaaaaaagatttgcgttttgatgacaaatatgatagatatataagttatttgggtagagtagaattagaaaatctgcctatattgtacttgcaaatgaatattttgtttatgcctacattgttggagacattctcaactacttatttggaggcaatgtttatgaaagtacctatagtagcatctgatatgagctttgcaagagacatttgtggtgaggcagccttatattgctctcctttaaatgaaaaagattatgcagaaaaaattttacttttgtataataataaagagctaaaaaatagtttgatttcaaaaggtcaaaaagaattgaaacgattcggagatagtatatcaagaacaaaaagatacttagaaataataaaaaaaacatctaatgaaaataaaaaataa

SEQ ID NO: 3 - DNA, 1272 bp, Capnocytophaga canimorsus
atgcaagctattaaaaataaatatggtaaaatattggcgtatatcgttgctattttgtatgtttcagatattccatttttcttcttaccgttcagtgtaagaactcgtttttttttaggtttgctagggatattcattttttttacatactatgtgcgcaatcgttgcttagacaaaagggtgtggaataatattttatggcttttagctctactgcttccttttttgttaacgactattgttaattctaatttagactcttggggttttagagccgtgcttaatgttttagtactatttgctgcatttttggttatacatttgttcaagaaaacagatattgaagctatttttcgtataataatcaacgttgttctaatcaatataatacttgcttatttaatgtttttcttttcacctatgaaagattttgtttttagattgcaagggttaaaagtcgatctaggggcacaagagtactatacaagcacttatagactttatggaattggaaattttttcttctatcaagcaggggtattttgttcttggatattaattcttatcacttattttttgaaaaaaaaagttacgctccatatggtattcacatatttttttgtgttgatatctggaatatttttcgcccgcacaacatttataggttttgtgatatctgttctgtattatttatttcctaatgcaggaacagtacaagttttcagacaatcatttctgaaagctatatttttagtttttactttgctaatacttttttctttgtcacaaaatatatttagaacatttttattagataataaagatgacaaaatgtttgcacatgcatttgaattttttttgtctttttatgaagatggaaagtttgaaacggattcaacaaatcatttgtctgaaatgtacatttatccagaatatttaaggacgtggttaataggagatggtaaattcgtggctgatggaggtgtgggttattatatgggaacagatgtaggatatttaagagtattattttttatgggtttatttggactatttgtgatgtatttgcaaaaaatatttttattatattctacttttaagctaaaaaagatagaccaatatagctataagctacttatatattttttgtttggacttacgttaatacttaacttgaagggatttacggatttggatatgcttgtttttttatttttttggcaacttttactcaataaagaagtggtgtacaatgaaaatattaatagataa

SEQ ID NO: 4 - DNA, 597 bp, Capnocytophaga canimorsus
atgaatagatttaaaaaatatagtattttaggaattatcgaattattatactttaaaataagaacttatttaacttttaaacaagctcgtataatacgatttccgtttagaattagaggaaaacaatttataaaaataggaaaaggttttacgacaggttatgattgtagaatcgatgcctttaatcattttagtagaaatggttttctgatagaaataggtgataacgtagagattaatgatagtgtacatattggtgctgtagaaaaagtaattatagaagataatgtgcttattgctagtaaagtatacatgtctgaccacaatcacggaagttataaaggagaggagcaagactctcctttaacaacacctaatagccgaaaaatatatactactcctatacatatcaaaaaaaatgtttggataggagagatggtatgcatactacaaggcgttacaataggagaaggttctatcattggagcgatgagtgtagtaaccaaagacattccgccttattcaatagccgttggctctccagcaagggtaattaagcagtttaattttgaaactaaaaagtgggaacaagtatag

SEQ ID NO: 5 - DNA, 1011 bp, Capnocytophaga canimorsus
atgattttaatagatgcactacatataaataatggaggaggaaaagtattattagattatcttgttgataaactcgaaaaaacaaatttagagctattttatctttttgatgatagaataaaaaaaacttacaatgtaaaacaaagcaaccaagctgtatatatgaattcttctattataaagagatataatttttatataaagtataagaatcgttttgcaaaaattttggttctaggtaatattcctcctctagtaaataccaatgcagaggtatatacctactttcataattctgtttatttgaatgttcctaatgatttcagttttatagaaggaacaaaatatgggttaaaagtaggtattatcaaatggatgagtccaaataccaattattggcttgtgcagtcagaaacagtaaaagaacaatttattaataaatttaggcaaagcaataaagtaaaagtgctcccgttttttccaaaattagaaagaactcaaggagtaaaaagagtgaaaagtacatttctttatgtaagtaatgctactcctcataaaaaccaccatagacttatagaagctttttgtcgagcttatgataaacttcagagagggaaacttattgttactgtagcagaacagtattctgacttgctgaagagactaaaatctatacaggacgaaggatatcccatagaaaatataggttttgtggataggttgactttagctagaaagtatcaggaatcggagtatattatttttccatctttagcggaaagttttggtttgggattagtggaaggaatatctatggggtgtaaggtattaggagctaatttaccttacacttttgcagtatgtgaaccttctttagtatttgacccaacaaatgttatagatatagagggagtaattatcaaagctatgtatagtgaattacctcaatctaaaactaaggtttataataaaatagatgaattgataagtatctttaagaattatgaatag

SEQ ID NO: 6 - DNA, 951 bp, Capnocytophaga canimorsus
atgaaagcacttttaaaaaaaatttacaaaagaacttggttaggtaaagttgccttacaaccatttcttaaaatatatcgttatttagagtataaactaatatctgatgagagttttctcaaacgaaaatttgaacaaacacaaggcttccctcttgatttgaaaaaccccaaaacccttaacgcaaaaatgcagtggctaaaaatcaatgatctaactcctttacatacgctttgcgccgacaaatatggtgtacgtgaatttgtacaaaaagaagtgggggcagagtatttagttcctgtactatttcggacaaataatcctaaggaactgacaaaagaaaatctgccacacaacactaactttattatcaaaacaacgcacgatagctctgggggagttattgttagaggagataaagagcttgacaaaattgattttcaattattacagaagaaatttacacatcttctcaaatccaaacattatcgaggaggaagagaaaatcagtacagagatatcaaacctcaaatcattgttgagcagttacttgaagataaaaaggggaaaataccaaatgattataaatttcattgctttaacggaaaggctaaggtggtatatgtttctgtcgatagggaaggtaagaataaacgtaatatctacgatattgattggaatcctctctattttacttggttggctcctgagaaagatgcctcgaacttgcgaggagaagagatagaaaaacctcaaaattacgaaaaaatgattcaaatcgcagagaaattagcttctcattttttgtatgttcgagttgatttgtacaatgttgagggtaaaatatatgtaggtgaattgacgtttcatcatggttcaggttttgaacatattcgtccaatagaatgggattataagatgggagaaatgttgaaattaccctttgaataa

SEQ ID NO: 7 - DNA, 951 bp, Capnocytophaga canimorsus
atgaaagcacttttaaaaaaaatttacaaaagaacttggttaggtaaagttgccttacaaccatttcttaaaatatatcgttatttagagtataaactaatatctgatgagagttttctcaaacgaaaatttgaacaaacccaaggcttccctcttgatttgaaaaaccccaaaacccttaacgcaaaaatgcagtggctaaaaatcaatgatctaactcctttacatacgctttgcgccgacaaatatggtgtacgtgaatttgtacaaaaagaagtgggggcagagtatttagttcctgtgctatttcggacaaataatcctaaggaactgacaaaagaaaatctgccacacaacactaactttattatcaaaacaacgcacgatagctctgggggagttattgttagaggagataaagagcttgacaaaattgattttcaattattacagaagaaatttacacatcttctcaaatccaaacattatcgaggaggaagagaaaatcagtacagagatatcaaacctcaaatcattgttgagcagttacttgaagataaaaaggggaaaataccaaatgattataaatttcattgctttaacggaaaggctaaggtggtatatgtttctgtcgatagggaaggtaagaataaacgtaatatctacgatattgattggaatcctctctattttacttggttggctcctgagaaagatgcctcgaacttgcgaggagaagagatagaaaaacctcaaaattacgaaaaaatgattcaaatcgcagagaaattagcttctcattttttgtatgttcgagttgatttgtacaatgttgagggtaaaatatatgtaggtgaattgacgtttcatcatggttctggttttgaacatattcgtccaatagaatgggattataagatgggagaaatgttgaaattaccctttgaataa

SEQ ID NO: 8 - DNA, 951 bp, Capnocytophaga canimorsus
atgaaagcacttttaaaaaaaatttacaaaagaacttggttaggtaaagttgccttacaaccatttcttaaaatatatcgttatttagagtataaactaatatctgatgagagttttctcaaacgaaaatttgaacaaacacaaggcttccctcttgatttgaaaaaccccaaaacccttaacgcaaaaatgcagtggctaaaaatcaatgatctaactcctttacatacgctttgcgccgacaaatatggtgtacgtgaatttgtacaaaaagaagtgggggcagagtatttagttcctgtactatttcggacaaataatcctaaggaactgacaaaagaaaatctgccacacaacactaactttattatcaaaacaacgcacgatagctctgggggagttattgttagaggagataaagagcttgacaaaattgattttcaattattacagaagaaatttacacatcttctcaaatccaaacattatcgaggaggaagagaaaatcagtacagagatatcaaacctcaaatcattgttgagcagttacttgaagataaaaaggggaaaataccaaatgattataaatttcattgctttaacggaaaggctaaggtggtatatgtttctgtcgatagggaaggtaagaataaacgtaatatctacgatattgattggaatcctctctattttacttggttggctcctgagaaagatgcctcgaacttgcgaggagaagagatagaaaaaccccaaaattacgaaaaaatgattcaaatcgcagagaaattagcttctcattttttgtatgttcgagttgatttgtacaatgttgagggtaaaatatatgtaggtgaattgacgtttcatcatggttcaggttttgaacatattcgtccaatagaatgggattataagatgggagaaatgttgaaattaccctttgaataa

SEQ ID NO: 9 - DNA, 31 bp, artificial sequence
  serotype A primer
aaaaaagtaccaatagtttttatatttaacc

SEQ ID NO: 10 - DNA, 31 bp, artificial sequence
  serotype A primer
tcatttttttatcttttttaatatattccac

SEQ ID NO: 11 - DNA, 43 bp, artificial sequence
  serotype A primer
cataccatgggaaaaaaagtaccaatagtttttatatttaacc

SEQ ID NO: 12 - DNA, 40 bp, artificial sequence
  serotype A primer
ccgctcgagtcatttttttatcttttttaatatattccac

SEQ ID NO: 13 - DNA, 19 bp, artificial sequence
  serotype B primer
attaacaaaattctaatag

SEQ ID NO: 14 - DNA, 20 bp, artificial sequence
  serotype B primer
ttattttttattttcattag

SEQ ID NO: 15 - DNA, 31 bp, artificial sequence
  serotype B primer
cataccatgggaattaacaaaattctaatag

SEQ ID NO: 16 - DNA, 29 bp, artificial sequence
  serotype B primer
ccgctcgagttattttttattttcattag

SEQ ID NO: 17 - DNA, 26 bp, artificial sequence
  serotype C primer
ggcgtatatcgttgctattttgtatg

SEQ ID NO: 18 - DNA, 29 bp, artificial sequence
  serotype C primer
ctattaatattttcattgtacaccacttc

SEQ ID NO: 19 - DNA, 33 bp, artificial sequence
  serotype D primer
gatttaaaaaatatagtattttaggaattatcg

SEQ ID NO: 20 - DNA, 27 bp, artificial sequence
  serotype D primer
ctatacttgttcccactttttagtttc

SEQ ID NO: 21 - DNA, 28 bp, artificial sequence
  serotype E primer
ggaggaggaaaagtattattagattatc

SEQ ID NO: 22 - DNA, 33 bp, artificial sequence
  serotype E primer
ctattcataattcttaaagatacttatcaattc

SEQ ID NO: 23 - DNA, 24 bp, artificial sequence
  serotype ABC primer
cttggttaggtaaagttgccttac

SEQ ID NO: 24 - DNA, 26 bp, artificial sequence
  serotype ABC primer
caacatttctcccatcttataatccc

SEQ ID NO: 25 - DNA, 900 bp, artificial sequence
  fragment of seq id 1
aaaaaagtaccaatagtttttatatttaaccaaggatatgctttaccagcgagtgtatgtattttttcattattacataataagaaagaggaagtaaattatcatatatgtattatgtatttaaggggacgactttcaagtcagactatttctaacataacagaaatggtttgtagttatggtagttgtagtatagagttcaaagaaatagaaaaccattttacaaataatagaactgtacgttatataactatcgatgcctactgtaagtttgaaataccaaaagtttttgaagattatgaaagagttatttacgcagatgtagatatgatttttgatggagatatatcagaattgatggatatatcttttaaggaatctattgcggcagtaagaattcctgtaggacacgtttccattgattttaaatcactcaatataaatgaagaaaattatttcaattctggattgatagtaatgaatacaaggaaaataaatgagaataagattattgaaaaaaaaatacgacctttgataggaaataaatatgaccacgtcgaccaagatatttttaatattgcatttaagggagatacttatttcttatcctctatatataacttctcttctgatagactcattaaagatagtgaagaacgtaacaagtctataaaggtatatcattacactggtgcaaagccttggaataaaggcgtatttccaatattttcagatatatggtgggaatattatagaagatctcctttctaccaagagaatgtttatttctcaaaacaatcagaatattatcagttacacagatatataaaattaggaactaagatagaaaaaatgggtgttttttctttgtggaatatattaaaaaagataaaaaaatga

SEQ ID NO: 26 - DNA, 1122 bp, artificial sequence
  fragment of seq id 2
attaacaaaattctaatagataattcaaatctttttgcaggtggaggtatacaagtggcaacatcttttttgaatgatttgaaagatatggatacttctccttacgtttttcatgtaatacaatcgtataatagcagtaaggctattgataaaacaaaatttcctaaagaattcatattttatgatttggaagaatctttgtcaaaaataaaaaaaatacaatacgtaaaagaaatagagaacaaagtcaaacccaattgtattttcactaccttcgggccatcttatcataagagtaattttccgaagattgttggttttgctttcggtcaaatgatttataaaaattcacccttttacaagaagctttcttttttgcaaaggctaaaatatgaatttattatttttatgaaaaaggagatgttccttaaaaattcagatgtacttatttttgaaacagatgacgctaaatttaaatttaatagattaacacataataagaaaatatcatatacggtaaataatacactgaatactatttttaccactccgaacgattggaaagattttaaaataaataaagtagggctgaatattctttgccttacggctaattatttacataaaaatctagatataattcctcgtgtaatagaccaacttcttacaatggataatgatttagattttaaattttttatttctttagaaaaaaaagatttgcgttttgatgacaaatatgatagatatataagttatttgggtagagtagaattagaaaatctgcctatattgtacttgcaaatgaatattttgtttatgcctacattgttggagacattctcaactacttatttggaggcaatgtttatgaaagtacctatagtagcatctgatatgagctttgcaagagacatttgtggtgaggcagccttatattgctctcctttaaatgaaaaagattatgcagaaaaaattttacttttgtataataataaagagctaaaaaatagtttgatttcaaaaggtcaaaaagaattgaaacgattcggagatagtatatcaagaacaaaaagatacttagaaataataaaaaaaacatctaatgaaaataaaaaataa

SEQ ID NO: 27 - DNA, 1233 bp, artificial sequence
  fragment of seq id 3
ggcgtatatcgttgctattttgtatgtttcagatattccatttttcttcttaccgttcagtgtaagaactcgtttttttttaggtttgctagggatattcattttttttacatactatgtgcgcaatcgttgcttagacaaaagggtgtggaataatattttatggcttttagctctactgcttccttttttgttaacgactattgttaattctaatttagactcttggggttttagagccgtgcttaatgttttagtactatttgctgcatttttggttatacatttgttcaagaaaacagatattgaagctatttttcgtataataatcaacgttgttctaatcaatataatacttgcttatttaatgtttttcttttcacctatgaaagattttgtttttagattgcaagggttaaaagtcgatctaggggcacaagagtactatacaagcacttatagactttatggaattggaaattttttcttctatcaagcaggggtattttgttcttggatattaattcttatcacttattttttgaaaaaaaaagttacgctccatatggtattcacatatttttttgtgttgatatctggaatatttttcgcccgcacaacatttataggttttgtgatatctgttctgtattatttatttcctaatgcaggaacagtacaagttttcagacaatcatttctgaaagctatatttttagtttttactttgctaatacttttttctttgtcacaaaatatatttagaacatttttattagataataaagatgacaaaatgtttgcacatgcatttgaattttttttgtctttttatgaagatggaaagtttgaaacggattcaacaaatcatttgtctgaaatgtacatttatccagaatatttaaggacgtggttaataggagatggtaaattcgtggctgatggaggtgtgggttattatatgggaacagatgtaggatatttaagagtattattttttatgggtttatttggactatttgtgatgtatttgcaaaaaatatttttattatattctacttttaagctaaaaaagatagaccaatatagctataagctacttatatattttttgtttggacttacgttaatacttaacttgaagggatttacggatttggatatgcttgtttttttatttttttggcaacttttactcaataaagaagtggtgtacaatgaaaatattaatag

SEQ ID NO: 28 - DNA, 590 bp, artificial sequence
  fragment of seq id 4
gatttaaaaaatatagtattttaggaattatcgaattattatactttaaaataagaacttatttaacttttaaacaagctcgtataatacgatttccgtttagaattagaggaaaacaatttataaaaataggaaaaggttttacgacaggttatgattgtagaatcgatgcctttaatcattttagtagaaatggttttctgatagaaataggtgataacgtagagattaatgatagtgtacatattggtgctgtagaaaaagtaattatagaagataatgtgcttattgctagtaaagtatacatgtctgaccacaatcacggaagttataaaggagaggagcaagactctcctttaacaacacctaatagccgaaaaatatatactactcctatacatatcaaaaaaaatgtttggataggagagatggtatgcatactacaaggcgttacaataggagaaggttctatcattggagcgatgagtgtagtaaccaaagacattccgccttattcaatagccgttggctctccagcaagggtaattaagcagtttaattttgaaactaaaaagtgggaacaagtatag

SEQ ID NO: 29 - DNA, 978 bp, artificial sequence
  fragment of seq id 5
ggaggaggaaaagtattattagattatcttgttgataaactcgaaaaaacaaatttagagctattttatctttttgatgatagaataaaaaaaacttacaatgtaaaacaaagcaaccaagctgtatatatgaattcttctattataaagagatataatttttatataaagtataagaatcgttttgcaaaaattttggttctaggtaatattcctcctctagtaaataccaatgcagaggtatatacctactttcataattctgtttatttgaatgttcctaatgatttcagttttatagaaggaacaaaatatgggttaaaagtaggtattatcaaatggatgagtccaaataccaattattggcttgtgcagtcagaaacagtaaaagaacaatttattaataaatttaggcaaagcaataaagtaaaagtgctcccgttttttccaaaattagaaagaactcaaggagtaaaaagagtgaaaagtacatttctttatgtaagtaatgctactcctcataaaaaccaccatagacttatagaagctttttgtcgagcttatgataaacttcagagagggaaacttattgttactgtagcagaacagtattctgacttgctgaagagactaaaatctatacaggacgaaggatatcccatagaaaatataggttttgtggataggttgactttagctagaaagtatcaggaatcggagtatattatttttccatctttagcggaaagttttggtttgggattagtggaaggaatatctatggggtgtaaggtattaggagctaatttaccttacacttttgcagtatgtgaaccttctttagtatttgacccaacaaatgttatagatatagagggagtaattatcaaagctatgtatagtgaattacctcaatctaaaactaaggtttataataaaatagatgaattgataagtatctttaagaattatgaatag

SEQ ID NO: 30 - DNA, 899 bp, artificial sequence
  fragment of seq id 6
cttggttaggtaaagttgccttacaaccatttcttaaaatatatcgttatttagagtataaactaatatctgatgagagttttctcaaacgaaaatttgaacaaacacaaggcttccctcttgatttgaaaaaccccaaaacccttaacgcaaaaatgcagtggctaaaaatcaatgatctaactcctttacatacgctttgcgccgacaaatatggtgtacgtgaatttgtacaaaaagaagtgggggcagagtatttagttcctgtactatttcggacaaataatcctaaggaactgacaaaagaaaatctgccacacaacactaactttattatcaaaacaacgcacgatagctctgggggagttattgttagaggagataaagagcttgacaaaattgattttcaattattacagaagaaatttacacatcttctcaaatccaaacattatcgaggaggaagagaaaatcagtacagagatatcaaacctcaaatcattgttgagcagttacttgaagataaaaaggggaaaataccaaatgattataaatttcattgctttaacggaaaggctaaggtggtatatgtttctgtcgatagggaaggtaagaataaacgtaatatctacgatattgattggaatcctctctattttacttggttggctcctgagaaagatgcctcgaacttgcgaggagaagagatagaaaaacctcaaaattacgaaaaaatgattcaaatcgcagagaaattagcttctcattttttgtatgttcgagttgatttgtacaatgttgagggtaaaatatatgtaggtgaattgacgtttcatcatggttcaggttttgaacatattcgtccaatagaatgggattataagatgggagaaatgttg

SEQ ID NO: 31 - DNA, 899 bp, artificial sequence
  fragment of seq id 7
cttggttaggtaaagttgccttacaaccatttcttaaaatatatcgttatttagagtataaactaatatctgatgagagttttctcaaacgaaaatttgaacaaacccaaggcttccctcttgatttgaaaaaccccaaaacccttaacgcaaaaatgcagtggctaaaaatcaatgatctaactcctttacatacgctttgcgccgacaaatatggtgtacgtgaatttgtacaaaaagaagtgggggcagagtatttagttcctgtgctatttcggacaaataatcctaaggaactgacaaaagaaaatctgccacacaacactaactttattatcaaaacaacgcacgatagctctgggggagttattgttagaggagataaagagcttgacaaaattgattttcaattattacagaagaaatttacacatcttctcaaatccaaacattatcgaggaggaagagaaaatcagtacagagatatcaaacctcaaatcattgttgagcagttacttgaagataaaaaggggaaaataccaaatgattataaatttcattgctttaacggaaaggctaaggtggtatatgtttctgtcgatagggaaggtaagaataaacgtaatatctacgatattgattggaatcctctctattttacttggttggctcctgagaaagatgcctcgaacttgcgaggagaagagatagaaaaacctcaaaattacgaaaaaatgattcaaatcgcagagaaattagcttctcattttttgtatgttcgagttgatttgtacaatgttgagggtaaaatatatgtaggtgaattgacgtttcatcatggttctggttttgaacatattcgtccaatagaatgggattataagatgggagaaatgttg

SEQ ID NO: 32 - DNA, 899 bp, artificial sequence
  fragment of seq id 8
cttggttaggtaaagttgccttacaaccatttcttaaaatatatcgttatttagagtataaactaatatctgatgagagttttctcaaacgaaaatttgaacaaacacaaggcttccctcttgatttgaaaaaccccaaaacccttaacgcaaaaatgcagtggctaaaaatcaatgatctaactcctttacatacgctttgcgccgacaaatatggtgtacgtgaatttgtacaaaaagaagtgggggcagagtatttagttcctgtactatttcggacaaataatcctaaggaactgacaaaagaaaatctgccacacaacactaactttattatcaaaacaacgcacgatagctctgggggagttattgttagaggagataaagagcttgacaaaattgattttcaattattacagaagaaatttacacatcttctcaaatccaaacattatcgaggaggaagagaaaatcagtacagagatatcaaacctcaaatcattgttgagcagttacttgaagataaaaaggggaaaataccaaatgattataaatttcattgctttaacggaaaggctaaggtggtatatgtttctgtcgatagggaaggtaagaataaacgtaatatctacgatattgattggaatcctctctattttacttggttggctcctgagaaagatgcctcgaacttgcgaggagaagagatagaaaaaccccaaaattacgaaaaaatgattcaaatcgcagagaaattagcttctcattttttgtatgttcgagttgatttgtacaatgttgagggtaaaatatatgtaggtgaattgacgtttcatcatggttcaggttttgaacatattcgtccaatagaatgggattataagatgggagaaatgttg

SEQ ID NO: 33 - DNA, 42 bp, artificial sequence
  fragment of seq id 2
atgattaacaaaattctaatagataattcaaatctttttgca

SEQ ID NO: 34 - DNA, 36 bp, artificial sequence
  fragment of Cc2 GT1 and Cc9 GT1
atgaaaatattaatagataattcaaacctttttgca

SEQ ID NO: 35 - DNA, 29 bp, artificial sequence
  wbuB6/9-KO1.1 primer
ggctgcagaagaatttccgagggctgatg

SEQ ID NO: 36 - DNA, 46 bp, artificial sequence
  wbuB6/9-KO1.2 primer
ctatgatgttgcaaataccgatgagcactttttatatttcttcgct

SEQ ID NO: 37 - DNA, 46 bp, artificial sequence
  wbuB6/9-KO3.1 primer
agcgaagaaatataaaaagtgctcatcggtatttgcaacatcatag

SEQ ID NO: 38 - DNA, 45 bp, artificial sequence
  wbuB6-KO3.2 primer
aaactcctgtgatgatggtttctacgaaggatgaaatttttcagg

SEQ ID NO: 39 - DNA, 45 bp, artificial sequence
  wbuB6-KO2.1 primer
cctgaaaaatttcatccttcgtagaaaccatcatcacaggagttt

SEQ ID NO: 40 - DNA, 30 bp, artificial sequence
  wbuB6/9-KO2.2 primer
ccactagtaggaatgcctttctctactatc

SEQ ID NO: 41 - DNA, 45 bp, artificial sequence
  wbuB9-KO3.2 primer
aagctcctgtgatgatgatttctacgaaggatgaaatttttcagg

SEQ ID NO: 42 - DNA, 45 bp, artificial sequence
  wbuB9KO2.1 primer
cctgaaaaatttcatccttcgtagaaatcatcatcacaggagctt

SEQ ID NO: 43 - DNA, 31 bp, artificial sequence
  wbtA12-KO1.1 primer
ggctgcagtcgcatattgggtacaactatag

SEQ ID NO: 44 - DNA, 47 bp, artificial sequence
  wbtA12-KO1.2 primer
ctatgatgttgcaaataccgatgagcttttggttagtataaaaatgg

SEQ ID NO: 45 - DNA, 47 bp, artificial sequence
  wbtA12-KO3.1 primer
ccatttttatactaaccaaaagctcatcggtatttgcaacatcatag

SEQ ID NO: 46 - DNA, 45 bp, artificial sequence
  wbtA12-KO3.2 primer
cctttcatagtatttaatattctacgaaggatgaaatttttcagg

SEQ ID NO: 47 - DNA, 45 bp, artificial sequence
  wbtA12-KO-2.1 primer
cctgaaaaatttcatccttcgtagaatattaaatactatgaaagg

SEQ ID NO: 48 - DNA, 28 bp, artificial sequence
  wbtA12-KO-2.2 primer
ccactagttctcttcgatggaaagtacg

SEQ ID NO: 49 - DNA, 1125 bp, artificial sequence
  C. canimorsus GT1
atgattaacaaaattctaatagataattcaaatctttttgcaggtggaggtatacaagtggcaacatcttttttgaatgatttgaaagatatggatacttctccttacgtttttcatgtaatacaatcgtataatagcagtaaggctattgataaaacaaaatttcctaaagaattcatattttatgatttggaagaatctttgtcaaaaataaaaaaaatacaatacgtaaaagaaatagagaacaaagtcaaacccaattgtattttcactaccttcgggccatcttatcataagagtaattttccgaagattgttggttttgctttcggtcaaatgatttataaaaattcacccttttacaagaagctttcttttttgcaaaggctaaaatatgaatttattatttttatgaaaaaggagatgttccttaaaaattcagatgtacttatttttgaaacagatgacgctaaatttaaatttaatagattaacacataataagaaaatatcatatacggtaaataatacactgaatactatttttaccactccgaacgattggaaagattttaaaataaataaagtagggctgaatattctttgccttacggctaattatttacataaaaatctagatataattcctcgtgtaatagaccaacttcttacaatggataatgatttagattttaaattttttatttctttagaaaaaaaagatttgcgttttgatgacaaatatgatagatatataagttatttgggtagagtagaattagaaaatctgcctatattgtactcacaaatgaatattttgtttatgcctacattgttggagacattctcaactacttatttggaggcaatgtttatgaaagtacctatagtagcatctgatatgagctttgcaagagacatttgtggtgaggcagccttatattgctctcctttaaatgaaaaagattatgcagaaaaaattttacttttgtataataataaagagctaaaaaatagtttgatttcaaaaggtcaaaaagaattgaaacgattcggagatagtatatcaagaacaaaaagatacttagaaataataaaaaaaacatctaatgaaaataaaaaataa

SEQ ID NO: 50 - DNA, 1122 bp, artificial sequence
  fragment of seq id 49
attaacaaaattctaatagataattcaaatctttttgcaggtggaggtatacaagtggcaacatcttttttgaatgatttgaaagatatggatacttctccttacgtttttcatgtaatacaatcgtataatagcagtaaggctattgataaaacaaaatttcctaaagaattcatattttatgatttggaagaatctttgtcaaaaataaaaaaaatacaatacgtaaaagaaatagagaacaaagtcaaacccaattgtattttcactaccttcgggccatcttatcataagagtaattttccgaagattgttggttttgctttcggtcaaatgatttataaaaattcacccttttacaagaagctttcttttttgcaaaggctaaaatatgaatttattatttttatgaaaaaggagatgttccttaaaaattcagatgtacttatttttgaaacagatgacgctaaatttaaatttaatagattaacacataataagaaaatatcatatacggtaaataatacactgaatactatttttaccactccgaacgattggaaagattttaaaataaataaagtagggctgaatattctttgccttacggctaattatttacataaaaatctagatataattcctcgtgtaatagaccaacttcttacaatggataatgatttagattttaaattttttatttctttagaaaaaaaagatttgcgttttgatgacaaatatgatagatatataagttatttgggtagagtagaattagaaaatctgcctatattgtactcacaaatgaatattttgtttatgcctacattgttggagacattctcaactacttatttggaggcaatgtttatgaaagtacctatagtagcatctgatatgagctttgcaagagacatttgtggtgaggcagccttatattgctctcctttaaatgaaaaagattatgcagaaaaaattttacttttgtataataataaagagctaaaaaatagtttgatttcaaaaggtcaaaaagaattgaaacgattcggagatagtatatcaagaacaaaaagatacttagaaataataaaaaaaacatctaatgaaaataaaaaataa